SEQUENCE LISTING (44 sequences)

SEQ ID NO: 1 - protein, 9 aa, Homo sapiens
LQNPRDVSV

SEQ ID NO: 2 - DNA, 27 bp, Homo sapiens
ctgcaaaacccacgtgacgtatctgtg

SEQ ID NO: 3 - protein, 9 aa, Homo sapiens
VVQVNTWSC

SEQ ID NO: 4 - DNA, 27 bp, Homo sapiens
gttgtccaagtgaacacttggtcttgc

SEQ ID NO: 5 - protein, 9 aa, Homo sapiens
YQPDQMRSA

SEQ ID NO: 6 - DNA, 27 bp, Homo sapiens
taccagccagaccagatgagatctgcg

SEQ ID NO: 7 - protein, 9 aa, Homo sapiens
IVGNERVQI

SEQ ID NO: 8 - DNA, 27 bp, Homo sapiens
atcgtggggaatgagagggtgcagata

SEQ ID NO: 9 - protein, 9 aa, Homo sapiens
RLWQELSDS

SEQ ID NO: 10 - DNA, 27 bp, Homo sapiens
cggctttggcaagagttgtctgattca

SEQ ID NO: 11 - protein, 9 aa, Homo sapiens
KLQERKKWQ

SEQ ID NO: 12 - DNA, 27 bp, Homo sapiens
aagctacaggagcggaagaagtggcag

SEQ ID NO: 13 - protein, 9 aa, Homo sapiens
QQLREQRKV

SEQ ID NO: 14 - DNA, 27 bp, Homo sapiens
caacagctgagagagcaaaggaaggtg

SEQ ID NO: 15 - protein, 9 aa, Artificial sequence
  Synthetic sequence amino acid sequence p11
RLWQELSDV

SEQ ID NO: 16 - DNA, 27 bp, Artificial sequence
  Synthetic sequence nucleic acid sequence p11
cggctttggcaagagttgtctgatgtc

SEQ ID NO: 17 - protein, 9 aa, Artificial sequence
  Synthetic sequence amino acid sequence p12
RLWQELSDL

SEQ ID NO: 18 - DNA, 27 bp, Artificial sequence
  Synthetic sequence nucleic acid sequence p12
cggctttggcaagagttgtctgatctc

SEQ ID NO: 19 - protein, 9 aa, Artificial sequence
  Synthetic sequence amino acid sequence p13
KLQERKKWV

SEQ ID NO: 20 - DNA, 27 bp, Artificial sequence
  Synthetic sequence nucleic acid sequence p13
aagctacaggagcggaagaagtgggtg

SEQ ID NO: 21 - protein, 9 aa, Artificial sequence
  Synthetic sequence amino acid sequence p14
RLWQELSDI

SEQ ID NO: 22 - DNA, 27 bp, Artificial sequence
  Synthetic sequence nucleic acid sequence p14
cggctttggcaagagttgtctgatatc

SEQ ID NO: 23 - protein, 9 aa, Artificial sequence
  Synthetic sequence amino acid sequence p15
LINPRDVSV

SEQ ID NO: 24 - DNA, 27 bp, Artificial sequence
  Synthetic sequence nucleic acid sequence p15
ctgataaacccacgtgacgtatctgtg

SEQ ID NO: 25 - protein, 9 aa, Artificial sequence
  Synthetic sequence amino acid sequence p16
LVNPRDVSV

SEQ ID NO: 26 - DNA, 27 bp, Artificial sequence
  Synthetic sequence nucleic acid sequence p16
ctggtaaacccacgtgacgtatctgtg

SEQ ID NO: 27 - protein, 9 aa, Cytomegalovirus
NLVPMVATV

SEQ ID NO: 28 - protein, 9 aa, Influenza virus
GILGFVFTL

SEQ ID NO: 29 - DNA, 31 bp, Artificial sequence
  Synthetic sequence pDOM forward primer
ttttaagcttgccgccaccatgggttggagc

SEQ ID NO: 30 - DNA, 60 bp, Artificial sequence
  Synthetic sequence p8 reverse primer
atatgcggccgcttagatatcagacaactcttgccaaagccggttaccccagaagtcacg

SEQ ID NO: 31 - DNA, 60 bp, Artificial sequence
  Synthetic sequence p14 reverse primer
atatgcggccgcttatgaatcagacaactcttgccaaagccggttaccccagaagtcacg

SEQ ID NO: 32 - DNA, 60 bp, Artificial sequence
  Synthetic sequence p15 reverse primer
atatgcggccgcttacacagatacgtcacgtgggtttatcaggttaccccagaagtcacg

SEQ ID NO: 33 - DNA, 60 bp, Artificial sequence
  Synthetic sequence p16 reverse primer
atatgcggccgcttacacagatacgtcacgtgggtttaccaggttaccccagaagtcacg

SEQ ID NO: 34 - DNA, 4109 bp, Homo sapiens
cagactttccgggcgcccaccaggctcccgggctctcaccggagaccacagggaggagcttcaaggtaccccaagtccctgcggcctccagcagtgaagagttccacaagggagtcttcctacgtcactgaataatgaatgaagatgagaggggaaaagagaagagacaaagtcaatccaaaaagctctcaaaggaaattaaactggattccatcatttcctacctatgattacttcaaccaagtgacgctacagttattagatggctttatgattacactgagcacagatggagtgatcatttgtgtggctgaaaacatctcttctcttcttggacatttaccagctgagattgtgggcaaaaaattattaagccttctgcctgatgaagagaaagatgaagtctaccaaaagattattctcaaatttcctttactaaactcagaaacacatattgaattttgctgtcatttaaaaagaggaaatgtcgaacatggtgatagttctgcttacgaaaacgtgaaatttattgtgaatgtaagagatatttgtaatgagtttcctgtggtctttagtggcttgttttccagccacctctgtgctgactttgctgcatgtgttcctcaggaggatcggctttatcttgtgggaaatgtttgcattctcaggactcagctcctgcagcaactttacacttcaaaggcagtcagtgatgaagctgtacttacacaagattcagatgaggaaccttttgtgggagagctcagtagctctcaaggtcaaagaggacacactagcatgaaagccgtgtacgttgaacccgctgctgctgctgctgctgctgctatctcagacgaccaaattgatattgcagaggttgagcagtatggaccacaagaaaacgttcacatgtttgtagattctgattcaacttattgctccagtacagttttcctggatactatgcctgaatctccagccttatccttgcaagactttcgaggtgagcctgaggtgaatccattgtacagggcagacccagtggacctggagttctcggtggatcaggtggactcagtggaccaggagggcccaatggaccagcaggacccagagaacccagttgccccgttggaccaggcaggcctgatggatccagtggatccagaggactcagtggacctgggggctgctggcgcaagtgctcagccattacagccatcatcaccagttgcatatgacatcattagccaggaactggaactgatgaagaagttgaaggagcagctagaagagaggacttggttgctgcatgatgccatccaaaaccagcagaatgcattggaattgatgatggatcaccttcagaagcagccaaacacattacgccacgttgtcattcctgatctccaatcttcggaggcagtgcccaagaaacaacagaaacaacacgctgggcaagtgaagcggcctctcccacatcccaaggacgtcaagtgtttctgtggcttatctttatccaactctctcaaaaacactggggagcttcaggagccttgtgttgccttcaaccagcagcaactggtgcagcaagaacaacacctgaaggagcagcagcggcagctgcgggagcagctgcaacagctgagagagcaaaggaaggtgcagaagcagaagaagatgcaggagaagaagaagctgcaggagcagaaaatgcaggagaagaagaagctgcaggagcagaggcggcaaaagaagaagaagctacaggagcggaagaagtggcaggggcagatgctacagaaagagccagaggaggagcagcagaagcagcagctgcaagagcagccactgaagcataatgtcatcgtggggaatgagagggtgcagatatgcctgcaaaacccacgtgacgtatctgtgcccctctgcaatcaccctgttagatttttacaggcccaacccattgttcctgtccagagagcagctgaacaacagccctctggcttctatcaagatgaaaactgtgggcaacaggaagatgagagtcaaaggtaagacatgcatggaatggtgatagtggctatgattattgcttttccctcatggctggatcccatgcctgagatcacagacagacgacctatccatgtggcaccagcgtccctctcatcaatagcaacttgctctcaacttgccttctctgtggccacggccatcatccttctgcatgtgtggtgcatggatgagcatccattgatggaacaaaggggtacccatgaagaaaggactagataaggaagcctcaatgctagggaattagcccacaaaccttgaaatcacttggctggattccccaagcccagtctcttgcacagggtgtctatggacaagtcttgtgcccatagctgaaagacttctgggcttccagtttcattccccaactttcctggaatctggtgatggcagtaggccaagtgcaggtatctccctggcatttatgaacagtaagagggaagtgagtgtgaaacaattgctagattgctaagatcagccaagaacacatgaaggctaagtcctgagtggcaatatagggacatgaacacatttctgctcaataataaggtcttcagttccaaatgctagaaagataaggttcttctcagcaatgaaaggattgcttagcacatagttggggcccactaaatagttttgaatgaatgttagctttctgagacgggcctcctaaagctctctggacttccttctgggccttgctttcgaatcagtgactttgacatgaagactgctttagggccatattgtccacttgacctgatcatcagaatccacctgaggagggtttggaaagtatagattcagagtctgggatggggttcaaaaatctgtattgatcacaagacttccaggcttagggttgtggagagagcactggatttggagtttggaaatctgccttacagtctctgctcagccattagctccctgagtgctttcaaagtaggtgaaatgactttccaaggccacagagagcatagttgtagcaggaacaagactgtccaagtaatataaataaaaccaggtgagacaagggattgtaatttctctcagtgtctacattcacattactttgacaagttgaatcttagtcaggccaaagtgagaatacaaacaagtgaatcctgtggtgtagccaccaaaaccaaatgtgcctgaagcacctactgatgaccacgtgagtgtctcatcagttatcatgtccagatccacatcactgcaacagctttcttctcttacagtttttatcctgaggcgtatcaagggccccccgtgaaccagctgccattgatagatacctcaaactctgaggcaatttcttcttccagcattcctcagtttcccataacttcagactcaaccataagcaccctggagaccccacaggattacatccggctttggcaagagttgtctgattcactcggtcctgttgtccaagtgaacacttggtcttgcgatgagcagggcaccctgcacggccaacccacctaccatcaggtgcaagtttctgaggtaggagtcgagggacctcctgatccacaggctttccaaggccctgctgcataccagccagaccagatgagatctgcggagcagaccagattgatgcctgcagagcaacgtgactcaaataagccgtgctaacagtactttcatgaccagtgatgaggggaaatggggggagggggcaggccaatgaggtctgcatggccaggggaccttcaaggtgcataaagtcccttggggtagggtttagtgggtagagacttgtttcctgataggttatgtttgtaattgtttgttaagcacagcctgtttcttggaagttatgctgtagaggcagcctgtgatccgtagtatgctagggtgtgacagcagccagccacagctggatctgatgtcttgtctgccccgcccagctttgcatatccatgttctaccacaggaaggtggcctgccaagagtctgctcaaagttttcaacataaagaataaagaaaaaaaaatgccaaaaaaaaaaaaaaaaaa

SEQ ID NO: 35 - protein, 639 aa, Homo sapiens
MKMRGEKRRDKVNPKSSQRKLNWIPSFPTYDYFNQVTLQLLDGFMITLSTDGVIICVAENISSLLGHLPAEIVGKKLLSLLPDEEKDEVYQKIILKFPLLNSETHIEFCCHLKRGNVEHGDSSAYENVKFIVNVRDICNEFPVVFSGLFSSHLCADFAACVPQEDRLYLVGNVCILRTQLLQQLYTSKAVSDEAVLTQDSDEEPFVGELSSSQGQRGHTSMKAVYVEPAAAAAAAAISDDQIDIAEVEQYGPQENVHMFVDSDSTYCSSTVFLDTMPESPALSLQDFRGEPEVNPLYRADPVDLEFSVDQVDSVDQEGPMDQQDPENPVAPLDQAGLMDPVDPEDSVDLGAAGASAQPLQPSSPVAYDIISQELELMKKLKEQLEERTWLLHDAIQNQQNALELMMDHLQKQPNTLRHVVIPDLQSSEAVPKKQQKQHAGQVKRPLPHPKDVKCFCGLSLSNSLKNTGELQEPCVAFNQQQLVQQEQHLKEQQRQLREQLQQLREQRKVQKQKKMQEKKKLQEQKMQEKKKLQEQRRQKKKKLQERKKWQGQMLQKEPEEEQQKQQLQEQPLKHNVIVGNERVQICLQNPRDVSVPLCNHPVRFLQAQPIVPVQRAAEQQPSGFYQDENCGQQEDESQR

SEQ ID NO: 36 - DNA, 3250 bp, Homo sapiens
agttccagagacagtagttatctcgggccccaacatcgcgaggtctagggaaaaatgctaccactcctcccccctacttccgctgccacgctgcctccatgcctgcctcccctctctgccacgcccccaccacgcgctttgcactctggggcccacgcacttccctgaagagtctagaagctgctcctcatttccagactttccgggcgcccaccaggctcccgggctctcaccggagaccacagggaggagcttcaaggtaccccaagtccctgcggcctccagcagtgaagagttccacaagggagtcttcctacgtcactgaataatgaatgaagatgagaggggaaaagagaagagacaaagtcaatccaaaaagctctcaaaggaaattaaactggattccatcatttcctacctatgattacttcaaccaagtgacgctacagttattagatggctttatgattacactgagcacagatggagtgatcatttgtgtggctgaaaacatctcttctcttcttggacatttaccagctgagattgtgggcaaaaaattattaagccttctgcctgatgaagagaaagatgaagtctaccaaaagattattctcaaatttcctttactaaactcagaaacacatattgaattttgctgtcatttaaaaagaggaaatgtcgaacatggtgatagttctgcttacgaaaacgtgaaatttattgtgaatgtaagagatatttgtaatgagtttcctgtggtctttagtggcttgttttccagccacctctgtgctgactttgctgcatgtgttcctcaggaggatcggctttatcttgtgggaaatgtttgcattctcaggactcagctcctgcagcaactttacacttcaaaggcagtcagtgatgaagctgtacttacacaagattcagatgaggaaccttttgtgggagagctcagtagctctcaaggtcaaagaggacacactagcatgaaagccgtgtacgttgaacccgctgctgctgctgctgctgctgctatctcagacgaccaaattgatattgcagaggttgagcagtatggaccacaagaaaacgttcacatgtttgtagattctgattcaacttattgctccagtacagttttcctggatactatgcctgaatctccagccttatccttgcaagactttcgaggtgagcctgaggtgaatccattgtacagggcagacccagtggacctggagttctcggtggatcaggtggactcagtggaccaggagggcccaatggaccagcaggacccagagaacccagttgccccgttggaccaggcaggcctgatggatccagtggatccagaggactcagtggacctgggggctgctggcgcaagtgctcagccattacagccatcatcaccagttgcatatgacatcattagccaggaactggaactgatgaagaagttgaaggagcagctagaagagaggacttggttgctgcatgatgccatccaaaaccagcagaatgcattggaattgatgatggatcaccttcagaagcagccaaacacattacgccacgttgtcattcctgatctccaatcttcggaggcagtgcccaagaaacaacagaaacaacacgctgggcaagtgaagcggcctctcccacatcccaaggacgtcaagtgtttctgtggtttatctttatccaactctctcaaaaacactggggagcttcaggagccttgtgttgccttcaaccagcagcaactggtgcagcaagaacaacacctgaaggagcagcagcggcagctgcgggagcagctgcaacagctgagagagcaaaggaaggtgcagaagcagaagaagatgcaggagaagaagaagctgcaggagcagaaaatgcaggagaagaagaagctgcaggagcagaggcggcaaaagaagaagaagctacaggagcggaagaagtggcaggggcagatgctacagaaagagccagaggaggagcagcagaagcagcagctgcaagagcagccactgaagcataatgtcatcgtggggaatgagagggtgcagatatgcctgcaaaacccacgtgacgtatctgtgcccctctgcaatcaccctgttagatttttacaggcccaacccattgttcctgtccagagagcagctgaacaacagccctctggcttctatcaagatgaaaactgtgggcaacaggaagatgagagtcaaagtttttatcctgaggcgtatcaagggccccccgtgaaccagctgccattgatagatacctcaaactctgaggcaatttcttcttccagcattcctcagtttcccataacttcagactcaaccataagcaccctggagaccccacaggattacatccggctttggcaagagttgtctgattcactcggtcctgttgtccaagtgaacacttggtcttgcgatgagcagggcaccctgcacggccaacccacctaccatcaggtgcaagtttctgaggtaggagtcgagggacctcctgatccacaggctttccaaggccctgctgcataccagccagaccagatgagatctgcggagcagaccagattgatgcctgcagagcaacgtgactcaaataagccgtgctaacagtactttcatgaccagtgatgaggggaaatggggggagggggcaggccaatgaggtctgcatggccaggggaccttcaaggtgcgtaaagtcccttggggtagggtttagtgggtagagacttatttgtttcctgataggttatgtttgtaattgtttgttaagcacagcctgtttcttggaagttatgctgtagaggcagcctgtgatccgtagtatgctagggtgtgacagcagccagccacagctggatctgatgtcttgtctgccccgcccagctttgcatatccatgttctaccacaggaaggtggcctgccaagagtctgctcaaagttttcaacataaagaataaagaaaaaaaaatgccaaagtgcttttcaatctagtaaatctagagggttgttttgtcttagccacaagaattccgaggtcttgaccctgatgatcaacctgcctcccctccatagtcttgttggagaagcccagagagaatgggactccaactaagggaacctgaaatcaactcaatggaggcacttcagagctaaaataattatggcttccttgcttaataaacattttcgttcactgcaa

SEQ ID NO: 37 - protein, 773 aa, Homo sapiens
MKMRGEKRRDKVNPKSSQRKLNWIPSFPTYDYFNQVTLQLLDGFMITLSTDGVIICVAENISSLLGHLPAEIVGKKLLSLLPDEEKDEVYQKIILKFPLLNSETHIEFCCHLKRGNVEHGDSSAYENVKFIVNVRDICNEFPVVFSGLFSSHLCADFAACVPQEDRLYLVGNVCILRTQLLQQLYTSKAVSDEAVLTQDSDEEPFVGELSSSQGQRGHTSMKAVYVEPAAAAAAAAISDDQIDIAEVEQYGPQENVHMFVDSDSTYCSSTVFLDTMPESPALSLQDFRGEPEVNPLYRADPVDLEFSVDQVDSVDQEGPMDQQDPENPVAPLDQAGLMDPVDPEDSVDLGAAGASAQPLQPSSPVAYDIISQELELMKKLKEQLEERTWLLHDAIQNQQNALELMMDHLQKQPNTLRHVVIPDLQSSEAVPKKQQKQHAGQVKRPLPHPKDVKCFCGLSLSNSLKNTGELQEPCVAFNQQQLVQQEQHLKEQQRQLREQLQQLREQRKVQKQKKMQEKKKLQEQKMQEKKKLQEQRRQKKKKLQERKKWQGQMLQKEPEEEQQKQQLQEQPLKHNVIVGNERVQICLQNPRDVSVPLCNHPVRFLQAQPIVPVQRAAEQQPSGFYQDENCGQQEDESQSFYPEAYQGPPVNQLPLIDTSNSEAISSSSIPQFPITSDSTISTLETPQDYIRLWQELSDSLGPVVQVNTWSCDEQGTLHGQPTYHQVQVSEVGVEGPPDPQAFQGPAAYQPDQMRSAEQTRLMPAEQRDSNKPC

SEQ ID NO: 38 - DNA, 1512 bp, Homo sapiens
gagcttcaggagccttgtgttgccttcaaccagcagcaactggtgcagcaagaacaacacctgaaggagcagcagcggcagctgcgggagcagctgcaacagctgagagagcaaaggaaggtgcagaagcagaagaagatgcaggagaagaagaagctgcaggagcagaaaatgcaggagaagaagaagctgcaggagcagaggcggcaaaagaagaagaagctacaggagcggaagaagtggcaggggcagatgctacagaaagagccagaggaggagcagcagaagcagcagttgcaagagcagccactgaagcataatgtcatcgtggggaatgagagggtgcagatatgcctgcaaaacccacgtgacgtatctgtgcccctctgcaatcaccctgttagatttttacaggcccaacccattgttcctgtccagagagcagctgaacaacagccctctggcttctatcaagatgaaaactgtgggcaacaggaagatgagagtcaaagtttttatcctgaggcgtatcaagggccccccgtgaaccagctgccattgatagatacctcaaactctgaggcaatttcttcttccagcattcctcagtttcccataacttcagactcaaccataagcaccctggagaccccacaggattacatccggctttggcaagagttgtctgattcactcggtcctgttgtccaagtgaacacttggtcttgcgatgagcagggcaccctgcacggccaacccacctaccatcaggtgcaagtttctgaggtaggagtcgagggacctcctgatccacaggctttccaaggccctgctgcataccagccagaccagatgagatctgcggagcagaccagattgatgcctgcagagcaacgtgactcaaataagccgtgctaacagtactttcatgaccagtgatgaggggaaatggggggagggggcaggccaatgaggtctgcatggccaggggaccttcaaggtgcgtaaagtcccttggggtagggtttagtgggtagagacttatttgtttcctgataggttatgtttgtaattgtttgttaagcacagcctgtttcttggaagttatgctgtagaggcagcctgtgatccgtagtatgctagggtttgacagcagccagccacagctggatctgatgtcttgtctgccccgcccagctttgcatatccatgttctaccacaggaaggtggcctgccaagagtctgctcaaagttttcaacataaagaaaaaagaaaaaaaaatgccaaagtgcttttcaatctagtaaatctagagggttgttttgtcttagccacaagaattccgaggtcttgaccctgatgatcaacctgcctcccctccatagtcttgttggagaagcccagagagaatgggactccaactaagggaacctgaaatcaactcaatggaggcacttcagagctaaaataattatggctaccttgcttaataaacattttcgttcactgc

SEQ ID NO: 39 - protein, 305 aa, Homo sapiens
ELQEPCVAFNQQQLVQQEQHLKEQQRQLREQLQQLREQRKVQKQKKMQEKKKLQEQKMQEKKKLQEQRRQKKKKLQERKKWQGQMLQKEPEEEQQKQQLQEQPLKHNVIVGNERVQICLQNPRDVSVPLCNHPVRFLQAQPIVPVQRAAEQQPSGFYQDENCGQQEDESQSFYPEAYQGPPVNQLPLIDTSNSEAISSSSIPQFPITSDSTISTLETPQDYIRLWQELSDSLGPVVQVNTWSCDEQGTLHGQPTYHQVQVSEVGVEGPPDPQAFQGPAAYQPDQMRSAEQTRLMPAEQRDSNKPC

SEQ ID NO: 40 - protein, 9 aa, Homo sapiens
KIQEQLQMV

SEQ ID NO: 41 - protein, 9 aa, Homo sapiens
FLTKGQQWI

SEQ ID NO: 42 - protein, 9 aa, Homo sapiens
VLQKSIDFL

SEQ ID NO: 43 - protein, 23 aa, Artificial sequence
  Synthetic sequence HLA class II-restricted p30
TTFNNFTVSFWLRVPKVSASHLE

SEQ ID NO: 44 - protein, 800 aa, Mus musculus
MINGMGDTPTDVKCSEDKGFPDRHASALLRQEEDFSISSITTLMERLCLALGNSIVKVAFDSILREKDDWSLWMDTETAPPVRRASNPSKRPTHEDLRNLLKKLQKMMDRLGYGPSRERPRALPTRYKSTIASGSSQRGMLSVQASYEHNEVPVFNTYEEYHKMALQEDIKGKSLLNFVLDEQKDEVSEKIILNLPLTSLVGSLIEFCCYIKKENVGQSGHGRHRYRAMYQGREIYEYVKFILYLQDSYDESFMFFGNCAHNRRNNRSSTSRLLWEQQYYLVGNISVLRTPDESAHPVKIQTNVIAVESNPIRQRRLLKRRQRSEMQRHAQAQAGVVDVEECPESGTEARPPSHSSISTEGSASTSISPCVRTSSSTPSTDTTTSTDITTTPAATPTSGQGYVIDPKNMLEPQDMEFEVGPEFVLIDSQDEQASLEQGEFTEADVKKPLEETKVSIIKEDVIDEDSARQGASSDDDDDSCIIIEDTEDNKDPKIQEATKVQEQGDQKAPDAVPSRPTTPLRAVRPEAVVEPIPRRTYQFHRPLLGERFAQLSGPRVMTQVYDLSISRSFHDELPSYIDMEEEDREQEQCEYELAQRIELLRNLPYERPGQQQSGQGPRVYRPREQMVTVIDDLGPRAMNFFGNSNSEYSQSEMHRRWETPVDHAPPLLHHSSRSCQEMAASHQALAAPCQASLPSCQVSLPSCQVSLPSRQVSLPSCQASLPSCQASALSRQASAVSRQPSTTSRQVSVSQQVAAAACYPSAMPHNASSDHGRENRPRFLPPGQDHAGYFQAEEDTNPHP

(19) United States
(12) Patent Application Publication
(10) Pub. No.: US 2011/0212116 A1
(43) Pub. Date: Sep. 1, 2011

(54) IMMUNOGENIC PEPTIDES AND USES THEREOF

(75) Inventors: Barbara Ann Guinn, London (GB); Nicola Rina,Mharie Hardwick, London (GB)

(73) Assignee: Cancer Research Technology Limited, London (GB)

(21) Appl. No.: 13/122,314

(22) PCT Filed: Oct. 1, 2009
(86) PCT No.: PCT/GB2009/002344

§ 371 Date: Apr. 1, 2011

(30) Foreign Application Priority Data
Oct. 2, 2008 (GB) 0818065.5

(51) Int. Cl. A61K 39/00; C12N 15/63; C12N 5/0783; C12Q 1/02; C07K 7/06; C07K 7/08; C07K 4/00; C07H 21/00; C12N 5/078; C12N 5/0784; C07K 16/18; C07K 14/725; C07K 14/74; C40B 30/04; A61P 35/00; A61P 37/04

(52) U.S. Cl. 424/185.1; 435/320.1; 435/325; 435/29; 530/328; 530/326; 530/300; 536/23.5; 530/387.9; 530/350; 506/9

(57) ABSTRACT

The present invention provides immunogenic peptides (and functional variants thereof) and their uses. The peptides comprise at least one PASD1-derived epitope. PASD1 is a cancer-testis antigen expressed in cancers, such as acute myeloid leukaemia (AML). Peptides of the invention are capable of inducing immune responses. The peptides are useful as vaccines.

### FIELD OF THE INVENTION

The present invention relates to malignancy-associated antigens. In particular, it relates to immunogenic peptides and nucleic acids encoding said peptides, as well as to vectors, cells, transgenic non-human organisms, vaccines and pharmaceutical compositions relating to such peptides and nucleic acids. The invention further relates to methods and uses of all of the products mentioned above.

### BACKGROUND OF THE INVENTION

Targeted immunotherapies require the identification and characterization of appropriate antigens. While initially T-cell based cancer vaccines were designed for patients with solid tumours, researchers extended the spectrum of cancer vaccines towards hematologic malignancies, for example acute myeloid leukaemia.

Acute myeloid leukaemia (AML) is a malignant clonal disorder of immature haematopoietic cells. The five year survival rates for patients under 60 years is 50%, but only 11% for patients over 60 years of age¹. Immunotherapy, in combination with conventional therapy, offers the opportunity to remove residual disease cells in first remission, thereby delaying and potentially preventing relapse.

The inventors previously used SEREX (serological analysis of recombinant cDNA expression libraries, reviewed in¹²) with minor modifications^{13,14} to immunoscreen a normal donor testis cDNA library to try and extend the number of cancer-testis (CT) antigens which have been identified in AML. CT antigens provide attractive targets for cancer specific immunotherapy. Their use avoids the concerns associated with targeting ‘self’ proteins, which may lead to autoimmunity and healthy tissue destruction. Although some CT antigens are expressed in some normal tissues, such as the testis and in some cases placenta, these immunologically-protected sites lack MHC class I expression and as such do not present ‘self’ proteins to the immune system. A number of CT antigens, such as HAGE¹⁵ have been found to be expressed in normal tissues, but their expression is less than 1/100 of the levels found in cancer cells. In addition, the targeting of what is described as selected non-essential tissues such as the breast is felt to balance out the risk of trying to improve current therapeutic treatments¹⁶. Many CT genes which were found to be expressed in solid tumours were found to have infrequent expression in myeloid leukaemias^{17,18}.

The immunoscreen of a testes cDNA library with four pooled M4 and M5 AML sera identified PASD1¹⁹, which is now established as one of the most frequently expressed CT antigens in presentation AML when compared to other CT antigens such as HAGE²⁰ (23%), BAGE²¹ (27%) and RAGE-1²² (21%). Like a number of other CT genes such as the MAGE-A1²³ and SAGE¹⁵ genes, PASD1 maps to the q28 region of chromosome X. See also WO03/082916. It was found that the region of PASD1 which the inventors had isolated encompasses about half of the region encoding PASD1_v1 as well as the region unique to _v2. This sequence was recognised by 35% of AML, 6% of CML and 10% of diffuse large B-cell lymphoma (DLBCL) but none of 18 normal donor sera¹⁹. In a recently published study, and with the help of present inventor Barbara Guinn, the OX-TES-1 cDNA was isolated following the immunoscreening of a testes library with DLBCL sera²⁶ and was found to encode a variant of PASD1 with a retained intron²⁴, which the authors called PASD1_v1. The retained intron leads to the translation of a shorter protein product due to a stop codon being introduced into the retained intron, which acts as an additional reading frame. The inventor's previous immunoscreening of OX-TES-1 with AML patient sera failed to detect any serum reactivity with the PASD1_v1 antigen²⁶; however the same sera used to screen GKT-ATA20 encoding the carboxy region of PASD1_v1 and the unique region of PASD1_v2 did show reactivity with approximately 10% of DLBCL sera. RT-PCR analysis indicated that PASD1 was expressed in 33% of AML patient samples. The inventors confirmed and quantitated their RT-PCR data using RQ-PCR. The inventors found PASD1 expression in the testis, placenta and pancreas by RT-PCR. This finding of PASD1 expression in the pancreas contrasts with the finding of Liggins et al²⁴ in which PASD1 expression was only found in the testes. However this study used Northern blot based techniques which are generally thought to be less sensitive than RT-PCR. Template from the testis and no other normal tissues were amplified in one round of RT-PCR suggesting a very low level of PASD1 expression in normal tissues (in this case pancreas) as observed with other CT genes^{15,23} and an increasing number of studies have also described the low level expression of CT antigens in the pancreas²⁵. Even if, despite the very low levels of PASD1 expression in normal pancreatic tissue, autoimmunity did occur against this organ through immunotherapy treatment targeting PASD1, the clinical effects would be expected to be treatable with the risk of auto-immunity against this single organ outweighing the life-threatening symptoms of AML.

Therapy targeting PASD1 has been shown to be applicable to a number of different tumour types including chronic and acute myeloid leukaemia¹⁹, diffuse large B-cell lymphoma²⁶ and multiple myeloma²⁷. A CTL response to PASD1 peptide in diffuse large B-cell lymphoma (DLBCL) has recently been reported⁴¹. PASD1 expression in primary solid tumours is being assessed but has already been shown in a range of solid tumour cell lines, including lung (H1299)¹⁹, head and neck (Hn5)¹⁹ and colon cancer (SW480)²⁴. Despite advances in the treatment of cancers, and haematological malignancies, such as AML in particular, there is a need for novel therapies to be developed. Indeed despite improvements in care and the advancements of stem cell transplants, in the last two decades, only 25% of AML patients remain alive at 5 years post-diagnosis.

### SUMMARY OF THE INVENTION

The present invention relates to epitopes of the cancer-testis antigen PASD1 together with associated nucleic acids and peptides. By combining computer based predictive methods with reverse immunology, the inventors have identified new PASD1-derived epitopes with affinity for HLA-A2. The inventors showed that these wild type peptides showed minimal binding to HLA-A2 on T2 cells but could induce IFN gamma secretion from normal donor T cells when stimulated with peptide loaded autologous dendritic cells. The inventors have gone on to develop anchor-modified analogue peptides and demonstrated that these peptides can bind MHC class I stably and for extended periods, and can induce epitope specific T cell responses from both normal donors and AML patient samples. Moreover, these T cells have been shown to recognise and lyse peptide loaded tumour target cells and tumour cells that have processed PASD1 endogenously.

One of the aims of these studies was to increase the arsenal of effective vaccines available, in order to allow a broader attack on human AML cells, thus minimizing the risk of antigenic escape from the immune system. The inventors explored, for example, the effectiveness of the pDOM-epitope design for the treatment of myeloid malignancies.

The inventors describe the identification of PASD1 epitopes, and in particular peptides comprising said epitopes and nucleic acid molecules encoding such peptides. In particular, the invention provides peptides which can induce PASD1-specific immune responses, in particular T cell specific immune responses, preferably HLA-A2 restricted T cell specific responses, in vivo and in vitro against processed and presented PASD1 epitopes in human cancer cells.

The epitopes/peptides described herein find utility as predictive, prognostic or diagnostic markers as well as therapeutic and prophylactic tools in the treatment of malignancies.

Thus, in one aspect the invention provides an immunogenic peptide of 8 to 50 amino acids in length comprising any one of SEQ ID NOs 21, 9, 15, 17, 19, 11, 23, 25, 1, 3, 5, 7 or 13.

In one aspect the invention provides an immunogenic peptide of 8 to 50 amino acids in length comprising at least one PASD1 epitope, wherein the epitope has the amino acids sequence of any one of SEQ ID NOs 9, 1, 3, 5, 7, 11 or 13 or a functional variant thereof.

Also provided is an immunogenic peptide of 8 to 50 amino acids in length comprising any one of SEQ ID NOs 9, 1, 3, 5, 7, 11 or 13 or a functional variant thereof. The peptide may be of 9 or 10 amino acids in length.

Also provided is an immunogenic peptide as described above, wherein the peptide is capable of stimulating a T cell response.

Also provided is an immunogenic peptide as described above, wherein the T cell response is a cytotoxic T cell (CTL) response.

Also provided is an immunogenic peptide as described above, wherein the T cell response is a T helper (TO cell response.

Also provided is an immunogenic peptide as described above, wherein the functional variant comprises at least one amino acid substitution compared to the parent sequence.

Also provided is an immunogenic peptide as described above, the peptide comprising any one of SEQ ID NOs 1, 3, 5, 7, 9, 11, 13, 15, 17, 19, 21, 23 or 25.

Also provided is an immunogenic peptide as described above, wherein the peptide comprises any one of SEQ ID NOs 21, 9, 15, 17, 19 or 11.

Also provided is an immunogenic peptide as described above, wherein the variant consists of the amino acid sequence of any one of SEQ ID NOs 15, 17, 19, 21, 23, 25.

Also provided is an immunogenic peptide as described above, wherein the peptide essentially consists of any one of SEQ ID NOs 1, 3, 5, 7, 9, 11 or 13.

Also provided is an immunogenic peptide as described above, wherein the peptide consists of any one of SEQ ID NOs 1, 3, 5, 7, 9, 11 or 13 with one amino acid substitution.

Also provided is an immunogenic peptide as described above, wherein the peptide essentially consists of the sequence of any one of SEQ ID NOs 1, 3, 5, 7, 9, 11, 13, 15, 17, 19, 21, 23 or 25.

Also provided is an immunogenic peptide as described above, wherein the peptide consists of SEQ ID NO 9 or SEQ ID NO 9 with one amino acid substitution.

Also provided is an immunogenic peptide consisting of SEQ ID NO 21, 9, 23, 25 or 1.

In one aspect of the invention, there is provided a polyepitope string comprising at least one of the epitopes as described herein, further comprising a further epitope, wherein the further epitope may be from the same or a different antigen.

In one aspect of the invention there is provided a nucleic acid encoding the peptide or the polyepitope string of the invention, respectively.

Also provided is a nucleic acid as described above, wherein the nucleic acid comprises any one of SEQ ID NOs 2, 4, 6, 8, 10, 12, 14, 16, 18, 20, 22, 24 or 26.

Also provided is a nucleic acid as described above, wherein the nucleic acid comprises any one of SEQ ID NO 22, 2, 10, 24, 26, 16, 18 or 20.

Also provided is a nucleic acid as described above, wherein the nucleic acid essentially consists of SEQ ID NO 22, 10, 24, 26, 2, 16, 18 or 20.

Also provided is a nucleic acid consisting of any one of SEQ ID NO 2, 4, 6, 8, 10, 12, 14, 16, 18, 20, 22, 24 or 26.

In one aspect of the invention there is provided an expression vector comprising the nucleic acid of the invention. The expression vector may be a pDOM plasmid.

In one aspect of the invention there is provided a coated particle comprising a peptide or a nucleic acid according to the invention.

In one aspect of the invention there is provided a transgenic non-human organism comprising a transgene capable of expressing an immunogenic peptide according to the invention.

In one aspect of the invention there is provided a cell comprising a peptide, a polyepitope string, a nucleic acid, a vector, or a particle of the invention, respectively. The cell may be an antigen presenting cell, for example a dendritic cell.

In one aspect of the invention there is provided a T cell or a T cell line which specifically recognises an epitope or peptide or polyepitope string as described herein.

In one aspect of the invention there is provided an agent capable of specifically binding an epitope or peptide or polyepitope string as described herein. The agent may be or comprise a T cell receptor or an antibody.

In one aspect of the invention there is provided a monomeric, tetrameric or pentameric complex comprising a multivalent MHC molecule and an epitope or peptide or polyepitope string as describe herein.

In one aspect of the invention there is provided a pharmaceutical composition comprising a peptide and/or a polyepitope string and/or a nucleic acid and/or an expression vector and/or a particle and/or a cell and/or a T cell and/or an agent and/or a complex of the invention, and a pharmaceutically acceptable carrier or diluent.

In one aspect of the invention there is provided a vaccine comprising a peptide and/or a polyepitope string and/or a nucleic acid and/or an expression vector and/or a particle and/or a cell and/or a T cell and/or an agent and/or a complex and/or a pharmaceutical composition of the invention, respectively, and optionally further comprising an adjuvant.

In one aspect of the invention there is provided a peptide, a polyepitope string, a nucleic acid, an expression vector, a particle, a cell, a T cell, an agent, a complex or a pharmaceutical composition of the invention, for use as a vaccine, or for use as an adjuvant.

In one aspect of the invention there is provided a use of a peptide, a polyepitope string, a nucleic acid, an expression vector, a cell, a T cell, and agent, a complex or a pharmaceutical composition of the invention, in prophylactic or therapeutic vaccination.

In one aspect of the invention there is provided a method of inducing an antigen-specific immune response in a subject, the method comprising delivering an effective amount of a peptide and/or a polyepitope string and/or a nucleic acid and/or an expression vector and/or a particle and/or a cell and/or a T cell and/or an agent and/or a complex and/or a pharmaceutical composition and/or a vaccine of the invention, to a subject.

In one aspect of the invention there is provided a peptide, a polyepitope string, a nucleic acid, an expression vector, a particle, a cell, a T cell, an agent, a complex, a pharmaceutical composition or a vaccine of the invention, for use as a medicament.

In one aspect of the invention there is provided a peptide, a polyepitope string, a nucleic acid, an expression vector, a particle, a cell, a T cell, an agent, a complex, a pharmaceutical composition or a vaccine according to the invention, respectively, for use in the treatment of cancer.

In one aspect of the invention there is provided a use of a peptide, a polyepitope string, a nucleic acid, an expression vector, a particle, a cell, a T cell, and agent, a complex, a pharmaceutical composition or a vaccine of the invention, respectively, for the manufacture of a medicament for the treatment of cancer.

In one aspect of the invention there is provided a method of treating cancer in a subject, the method comprising administering to a subject in need thereof a therapeutically effective amount of a peptide and/or a polyepitope string and/or a nucleic acid and/or an expression vector and/or a particle and/or a cell and/or a T cell and/or an agent and/or a complex and/or a pharmaceutical composition and/or a vaccine of the invention, respectively.

In one aspect of the invention there is provided a method of generating an immunogenic variant peptide, the method comprising
(i) obtaining a parent peptide, the parent peptide comprising at least one copy of a subsequence of PASD1, wherein the subsequence is any one of SEQ ID NOs 1, 3, 5, 7, 9, 11 and 13,
(ii) modifying the subsequence of the parent peptide by substitution, deletion or insertion of one or more amino acids, and
(iii) testing the variant peptide of (ii) for immunogenicity.

In one aspect of the invention there is provided a method of detecting a cancer, the method comprising testing a sample obtained from a subject for the presence of
(a) a T cell or T cell line specific for a peptide of the invention, or
(b) an epitope or peptide of the invention, or
(c) an APC or tumour cell presenting an epitope or peptide of the invention on an MHCI molecule, or
(d) a TCR recognising the epitope or peptide of the invention, or
(e) activation of T cells (i.e. detection of IFNγ production) against the epitope or peptide of the invention
(f) peptide-specific T cells using the pMHC array.

In one aspect of the invention there is provided a method of predicting the susceptibility of a subject for a treatment as described in claim 48 or 49, the method comprising testing a sample obtained from a subject for the presence of
(a) a T cell or T cell line specific for a peptide of the invention, or
(b) an epitope or peptide of the invention, or
(c) an APC or tumour cell presenting an epitope or
(d) peptide of the invention on an MHCI molecule, or
(e) a TCR recognising the epitope or peptide of the invention, or
(f) (f) detection of peptide-specific T cells using an pMHC array,
   wherein detection of any one of features (a) to (f) indicates the subject's susceptibility for said treatment.

In one aspect of the invention there is provided a method of monitoring an anti-PASD1 immune response in a subject which comprises detecting in a sample obtained from the subject the presence of:
1) an epitope or peptide or polyepitope string according to claims 1-19 (see peptide claims), or
2) a T cell or a T cell line as according to claims 31-33
3) a T cell receptor according to claim 35 and/or,
   wherein the presence of said epitope, peptide, polyepitope string, T cell, T cell line or T cell receptor indicates an anti-PASD1 immune response.

In one aspect of the invention there is provided a method of producing an anti-serum against an antigen, said method comprising introducing a peptide of the invention, or a fragment thereof, or a polyepitope string of the invention, or a nucleic acid of the invention, an expression vector of the invention, a particle of the invention or a cell or T cell of the invention into a non-human mammal, and recovering immune serum from said mammal. Also provided is an antibody obtainable from said serum.

The invention will now be described with reference to the figures described below.

### DESCRIPTION OF THE FIGURES AND TABLES

FIG. 1. Modification of the wild type PASD1 peptides led to increased MHC class I binding and IFNγ secretion by responding autologous T cells. (A) Stabilisation of HLA-A2 molecules on the surface of T2 cells after overnight incubation with peptides (wild type CLOCK peptides (P1-P3), wild type PASD1 peptides (P4-P10) or modified PASD1 peptides (P11-P16)) at 50 μmolar. T2 cells incubated with FLU were included for comparison. Staining with the isotype control antibody is shown in green, and staining with HLA-A2-FITC in pink for the wild type peptides, while staining with isotype control is shown in black and staining with HLA-A2-FITC is shown in red for the PASD1 modified peptides. (B) IFNγ secretion was measured from healthy donor T cells stimulated with peptide loaded autologous dendritic cells. CD3⁺ T cells were stimulated with autologous DCs loaded with wt peptides 1-10 (P1-3 were used as wild type controls and are located in the human CLOCK gene, the gene currently believed to be closest in sequence to PASD1), CMV/FLU or no peptide control. Aliquots of culture supernatant were collected on days 3, 7, 10 and 14 and analysed for IFNγ levels by ELISA. IFNγ at different time points is expressed in pg per 10⁶ effector cells. (C) The duration of PASD1 peptide analogue binding to HLA-A*0201 molecules was determined using a modified T2 assay. T2 cells were incubated with peptide, washed three times and replated in fresh medium. Samples were taken at various time points after removal of peptide and FACS analysis for HLA-A2 levels carried out. In particular, the HLA-A2⁺T2 cell line⁴⁵ was used to assess binding of peptides to HLA-A2. T2 cells were incubated overnight in complete media (RPMI1640, 1 mM sodium pyruvate, 2 mM L-glutamine, 1% non-essential amino acids, 50 μM2-mercaptoethanol, 100 U/ml penicillin, 100 μg/ml streptomycin; all Invitrogen) with 10% FCS alone or with peptide (0.05-100 μM) prior to staining with anti-human HLA-A2-FITC antibody and FACs analysis. To determine the longevity of binding, peptide-pulsed T2 cells were washed serum-free three times and replated in fresh medium. Aliquots of cells were analysed at different time points after the removal of peptide by flow cytometry. Plot shows mean fluorescence (y-axis) against time after removal of peptide in hours (x-axis). Data obtained from P6 were representative of the P4-P10 wild type peptides.

FIG. 2. Modified peptides can induce IFNγ secretion by responding normal donor CD8+ T cells. (A) IFNγ levels in T cell cultures stimulated with autologous DCs which were loaded with either peptide analogues P11-16, or CMV or FLU peptide which acts as positive controls, or no peptide which acts as a negative control. Culture supernatants were collected at different time points after stimulation and IFNγ levels in the supernatant were determined by ELISA. Six healthy donors were tested. CMV and FLU controls were included for each donor, but only one is shown on each plot for reference. IFNγ levels are expressed as pg IFNγ per 10⁶ cells. (B) Bar charts show levels of IFNγ in cultures of CD4⁺ depleted/non-depleted effector 20″ cells stimulated with peptide analogues P14, P15, P16, CMV and no peptide control. IFNγ levels are expressed as pg IFNγ per 10⁶ cells. (C) FACS plots show intracellular IFNγ FITC. Staining of CD3⁺ cells after three stimulations with one of the peptide analogues in this case P14. Cells were co-stained with PE labelled antibodies to CD4⁺ or CD8⁺ in order to determine the phenotype of the IFNγ secreting cells. Data showed that the IFNγ secreting cells were CD8+ although cultures were dependent on CD4⁺ help for the CD8⁺ response.

FIG. 3. PASD1 specific T cells were identified in populations of peptide stimulated primary cells. (A+E) Healthy Donor I and healthy donor II, respectively; CDT3⁺ cells stimulated with autologous DCs alone or DCs loaded with peptide. No pentamer positive cells were detected after one, two or three stimulations. FACS plots show pentamer-PE (FL-2) against CD8-FITC (FL-1) staining after four stimulations. (B+E) AML Patient I and AML Patient II, respectively; CD3+ cells stimulated with peptide loaded T2 cells. FACS plots show cells stained with PE labelled pentamers (FL-2) and CD8-FITC (FL-1) after two stimulations. Further stimulation led to activation induced cell death. (C) Colon cancer patient VI showed an increase in P14-specific T cells after three rounds of stimulation which (D) were further increased after four rounds of peptide stimulations. Pentamer positive cells are expressed as the frequency of pentamer⁺ CD8⁺ T cells as shown on histogram. X-axis indicates pentamer-PE staining (FL2-H), while the Y-axis indicates CD8-FITC staining (FL1-H).

FIG. 4. IFNγ secretion by patient T cells following in vitro stimulations. (A) IFNγ levels in peptide stimulated cultures from AML patients (same as shown in FIGS. 3 B and E) as measured by ELISpot assays. (B) IFNγ levels in T cell cultures from HLA-A*0201 patients with colon cancer stimulated with autologous DCs cells alone, or pulsed with peptide analogues P14, P15, FLU and no peptide negative control. Graphs show IFNγ levels at day 10 (y-axis).

FIG. 5. Immunisations of HHD mice demonstrate that the modification of the P8 peptide is essential for highly effective immune responses. (A) The pDOM epitope vaccine consists of a DNA plasmid backbone incorporating CpG sites. The first domain of tetanus toxin (DOM; TT865-1120) is used to provide tumour specific antibody, CD4+ and CD8+ responses when linked to a tumour associated nucleotide sequence, encoding the peptide of interest. This format allows the appropriate processing and presentation of the peptide, as well as simultaneous stimulation of CD8+ cells by the epitope inserted CD4+ stimulation by the promiscuous CD4 epitope p30 within the 1^{st} domain (DOM) of tetanus toxin⁸ and the proposed stimulation of the innate immune response by CpGs in the DNA vaccine backbone. (B) In priming experiments mice were immunised and 14 days later ELISpot assays were performed on individual mice which had been injected with P14 (labelled P14-1 to P8-6), P15 (labelled P15-1 to P15-6), P16 (labelled P16-1 to P16-6), WT1 (irrelevant control; WT-1 and WT-2) or pDOM alone (labelled pDOM-1 to pDOM-3). Responses shown in red are to P14 peptide, the same as the initial immunisation, while the yellow columns indicate responses by splenocytes from the same mice to the wild type epitope P8. The data demonstrate that only P14 immunised mice elicit IFNγ secretion against both the modified P14 peptide they were immunised with and the P8 wild type peptide, which P14 is derived from. pDOM responses in P15 and P16 immunised mice demonstrate the operation integrity of the pDOM-epitope vaccine in these mice despite an absence of P15 and P16 responses. (C) In priming experiments mice were immunised with either P8, P14 or pDOM, and 14 days later ELISpot assays were performed on individual mice injected with P8 (labelled P8-1 to P8-4), P14 (labelled P14-1 to P14-6) or pDOM alone (labelled pDOM-1). Responses shown are to the same peptide as the initial immunisation and demonstrate that priming with P14 leads to an IFNγ response, while priming with P8 does not. (D) CTL assays on mice which had been injected with the pDOM.P14 DNA vaccine could kill peptide loaded P8 and P14 targets equally well, indicating that the modified peptide still induced effective T cell responses against the wild type peptide. This figure also shows that killing of P15 or P16 loaded targets by P15 or P16-vaccinated mice did not occur.

FIG. 6. CTL lines from pDOM.P14 immunised mice can lyse peptide loaded and endogenously processed wild type P8 peptide. Following priming and boosting with EP using pDOM.P14 at both treatments, splenocytes were taken and repeated ex vivo stimulations with 1 μM of P8 peptide were performed until the CTL lines began to expand (approximately three-fold). Lysis of peptide-loaded targets were assessed using a 5 hr ⁵¹Chromium-release assay and we show that P14 prime and boosted T cells which were expanded ex vivo with P8 could lyse (A) P8 loaded targets (K562 cells, which are HLA-A2 negative but have been modified to express the HHD molecule a mouse/human MHC-class I hybrid molecule which T cells from HHD mice can recognise); (B) endogenously processed antigen in K562 cells which have been transduced with HHD (C) endogenously processed PASD1 in A2 positive cells (SW480) but not A2 negative cells (K562 with HHD expression). Data shows that P14 immunised mice can recognise endogenously processed PASD1 (P8) presented on human MHC class I on cancer cells.

FIG. 7: Nucleotide and amino acid sequence for each PASD1 variant

FIG. 8: Mapping of immunogenic peptides on the PASD1 sequence. FIGS. 8A and 8B: Genomic structure of PASD1_v1 (A) and PASD1_v2 (B). Exons are indicated as open boxes, introns as lines and the retained intron in PASD1_v1 is indicated with a black box, with the tga indicating the site of the premature stop signal, which leads to the shorter PASD1a protein. The position of predicted translation start (atg) and stop (tga) sites are indicated for both variants. The approximate region within which the epitopes described herein reside are indicated by the dotted line. This is the region pulled out from the testes library following immunoscreening with AML sera. FIG. 8C: The PASD1_v1 and PASD1_v2 proteins (also referred to as PASD1a and PASD1b proteins, respectively), along with the murine homologue, mPASD1 are shown. Identical residues are highlighted while similar residues are shaded in grey. The murine protein shows 35.7% similarity (25.2% identity) with PASD1_v1 and 34.1% similarity (24.2% identity) with PASD1_v2²⁴. The location of wild type peptides identified by the inventor are shown in coloured outlined boxes.

FIG. 9: Lysis of HHD-transduced or HLA-A2-positive human cancer cells by CTL lines from p.DOM-P14 immunised mice. Two weeks following the vaccination of HHD mice with p.DOM-P14, splenocytes were stimulated in vitro with 1 μM of P14 peptide on a weekly basis. (A) HLA-A2 expression in K562 cells which were transduced with either the MSCV retroviral vector alone (K562-RV) or the MSCV-HHD retrovirus (K562-HHD). Single black lines indicate the expression detected by the HLA-A2 antibody, grey line indicates isotype control and light grey cells alone. (B) Lysis of K562 and its retrovirally transduced variants, as well as P8 (indicated as Pw8) loaded K562-HHD by Pa14 stimulated CTL lines are shown. In addition, CTL lines could lyse endogenously processed P8 (indicated as Pw8) peptide from PASD1 within the K562-HHD cells. K562 is a NK sensitive cell line, but little or no lysis of the K562 alone is seen indicating that NK lysis in this assay was minimal.

FIG. 10: (A) SW480 cells naturally express HLA-A2 and (B) were lysed by CTL lines from HHD mice which had been immunised with p.DOM-P14. Blocking of HLA-A2 with the anti-HLA-A2 antibody W6/32 abrogated CTL lysis of the SW480 cells. CTL activity was measured using a 5 hr ⁵¹Cr-release assay.

FIG. 11: pMHC arrays. pMHC molecules were folded into tetramers using either streptavidin alone or AlexaFluor 532 (Molecular Probes) conjugated to streptavidin. Tetramers were spotted onto hydrogel slides using a contact deposition-type printer (Genetix), at a concentration of 0.5 mg/ml in 2% glycerol. Printed arrays were immobilised for 48 hours and stored at 4° C. until use. (FIG. 11 i) CD8+ T cells were negatively isolated from normal donor buffy coats obtained from National Blood Service UK or patient samples from the Department of Haematology, Southampton General Hospital following informed consent using EasySep isolation kits. Cells were labelled with DiD (Molecular Probes) according to the manufacturer's instructions. The selected array was warmed to room temperature and incubated with labelled CD8+ cells (10⁶/ml) in colourless X-VIVO 15 for 20 minutes at 37° C. Unbound cells were washed away with warm colourless X-VIVO 15. Excess culture medium was removed before slides were analysed on the ProScanArray (PerkinElmer). (FIG. 11 ii) FACS analysis was used to confirm T-cell populations recognising specific epitopes. Negatively isolated CD8+ T cells were labelled with CD8-FITC (FL1-H) and pMHC-SAPE (FL2-H) and analysed by flow cytometry using the FACScalibur™. In this normal donor a small population of FLU M1-specific T cells was detected, but no CMV pp65 specific T cells. (FIG. 11 iii) On custom-made hydrogel slides CD8+ T cells from the same normal donor (shown stained red) are visible at the single cell level bound to the Flu M1 tetramer (shown in green) but not the CMV pp65 tetramer. Composites show the co-localisation of Flu-specific CD8+ T cells bound to tetramer spots from a HLA-A2 +, Flu M1 +, but not to the CMV pp65 or random tetramer negative control spots.

FIG. 12: Analysis of patient samples. The inventors examined CD8 T cells negatively purified from (a) 11 patients with myeloid leukaemia at the time of disease presentation on the pMHC array. No peptide expansion of the T cells was performed ex vivo prior to analysis. Positives were only scored where at least 3 of 9 spots had T cells stuck to them and this occurred in both sets of spots in independent locations on the array. Of the 7 AML patients four were known to be HLA-A2 positive. The inventors found that (b) two of the known HLA-A2 positive AML patients had T cells (visibly as a yellow colour) which bound to PASD1 P14 (shown as Pa14) tetramers while (c) the other two HLA-A2 positive AML patients did not. Controls of AF532 fluorochrome alone and random tetramer in all patients examined were negative. Negative controls of AF532 and random tetramer are shown for patient 3857506 (d+e, respectively).

Table 1: Patient characteristics
Table 2: Mapping of the PASD1 epitopes, wild type P4-P10 and single amino acid modified P11-P16.

### DETAILED DESCRIPTION OF THE INVENTION

Despite improvements in the treatment and care of patients with acute myeloid leukaemia (AML), novel therapies need to be developed to increase the survival rates in this disease which is difficult to treat. Immunotherapy has the potential to remove residual AML cells in first remission, extending this phase and ideally preventing relapse.

The inventors have previously identified the cancer-testis antigen PASD1 through immunoscreening of a testes library with pooled AML sera and isolated what was predominantly the unique region of the cDNA subsequently described by Liggins et al²⁴ as PASD1 variant 2. As describe above, two splice variants exist: PASD1_v1 (nucleotide sequence: SEQ ID NO 34, Accession number AY270020, amino acid sequence: SEQ ID NO 35, Accession number AAQ01136.1) and PASD1_v2 (nucleotide sequence: SEQ ID NO 36, Accession number NM_{—}173493, amino acid sequence: SEQ ID NO 37, Accession number NP_{—}775764.2).

The inventors have now used web-based algorithms (SYFPEITHI and BIMAS) and reverse immunology to identify HLA-A*0201 binding epitopes within PASD1 (PASD1_v1 and/or PASD1_v2). In silico methods, however, cannot predict that peptides are correctly processed and/or presented. Peptides were only further investigated if the peptide did not map to any other known eukaryotic proteins except PASD1. Peptides were ordered from ProImmune and their binding to HLA-A2 and their binding/immunogenicity were tested in various ways. (1) By incubating 50 μM of peptide with T2 cells overnight, washing off excess peptide and then performing FACs analysis to assess binding by virtue of stabilised HLA-A2 expression on T2 cells. (2) By examining the ability of the peptides to induce T cells responses (as measured by IFNγ ELISA assays) when loaded onto autologous normal donor monocyte-derived dendritic cells in mixed lymphocyte reactions, and (3) by examining T cell expansion following stimulation of T cells from normal donors and patients with antigen presenting cells loaded with peptide using pentamers.

The inventors tested wildtype peptides for immunogenicity. The inventors also modified binding residues within the peptides (Table 2) and re-assessed immunogenicity.

The inventors were able to identify peptides that bound well to HLA-A2 for extended periods and induced IFNγ production when T cells were stimulated with peptide loaded antigen presenting cells. In addition a notable expansion of PASD1-specific T cells were observed in patient samples stimulated with peptides of the invention. Of particular note was the expansion of T cells from a patient with colon cancer following 3 and 4 stimulations with antigen presenting cells loaded with a particular peptide, P14—a derivative of ‘wildtype’ peptide P8. HHD studies using mice showed that P14 was effective in stimulating T cells which could kill tumour cells which were either loaded with the wild type P8 peptide or which endogenously processed the PASD1 antigen.

The authors have shown for the first time that PASD1 containing vaccines, for example DNA vaccines, may be used to induce effective T cell responses which can induce specific T cell responses and lead to the killing of tumour cells.

An effective (adaptive) immune response involves two major groups of cells: lymphocytes and antigen presenting cells. The two major populations of lymphocytes are B cells and T cells. There are two well-defined subpopulations of T cells: T helper (TH) and cytotoxic T (TC) cells. TH and TC can be distinguished from one another by the presence of either CD4 or CD8 membrane glycoproteins on their surface. T cells displaying CD4 generally function as TH cells, whereas those displaying CD8 generally function as TC cells. TC cells can develop into cytotoxic T lymphocytes (CTLs) that exhibit cytotoxic activity. T cells carry T cell receptor (TCR). Most TCR recognise antigen only when it is bound to major histocompatibility complex (MHC) molecules. There are two major types of MHC molecules: MHC class I molecules, which are expressed by nearly all nucleated cells of vertebrate species, and MHC class II molecules, which are expressed only by antigen presenting cells (APCs).

T cells that recognize only antigenic peptides displayed with a MHC class II molecule generally function as TH cells. T cells that recognize only antigenic peptides displayed with a MHC class I molecule generally function as TC cells. The MHC in humans is termed the Human Leukocyte Antigen system (HLA), HLA class I (A, B and C), which is generally associated with stimulation of CTLs, and HLA class II (DR, DP and DQ), which is generally associated with stimulation of T_{H} cells. HLA-A*0201 presents the most common A2 serotype with 45% of Caucasians expressing this HLA class I molecule.

### Peptides

The inventors were able to identify peptides that bound well to HLA-A2 for extended periods and induced IFNγ production when T cells were stimulated with peptide-loaded antigen presenting cells. In particular, the invention provides peptides, preferably immunogenic peptides. Such peptides may comprise a PASD1 subsequence or a functional variant thereof. The subsequence or functional variant thereof is preferably 9 amino acids long.

Thus, in a first aspect the invention provides peptides. In particular, the invention provides immunogenic peptides, i.e. the peptides are capable of eliciting an immune response in an organism. Preferably, the peptides of the invention are capable of eliciting a specific T cell immune response, such as a cytotoxic T cell response or a T helper cell response. For example, the peptides of the invention are capable of eliciting a HLA-A2 restricted T cell response.

The peptides of the invention are capable of binding to a major histocompatibility complex (MHC) molecule (class I and/or class II), preferably to a Human Leukocyte Antigen (HLA) molecule. Preferably, the peptides of the invention can bind to a MHC class I molecule. Preferably, they can bind to HLA-A, more preferably to HLA-A2, and more preferably to HLA-A*0201.

The peptides of the invention may be of from 8 to 50 amino acids in length, more preferably of from 8 to 40 amino acids, more preferably 8 to 30 amino acids, more preferably 8 to 25 amino acids, more preferably 8 to 20 amino acids. For example, the peptides may be 9-50, or 9-25 amino acids in length. For example, a peptide of the invention may be 8, 9, 10, 11, 12, 13, 14, 15, 16, 17, 18, 19, 20, 21, 22, 23, 24 or 25 amino acids long. Preferably the peptides of the invention are 9 or 10 amino acids long, most preferably they are 9 amino acids long. The peptide can be extended or shortened on either the amino or the carboxyterminal end or internally, or extended on one end and shortened on the other end, provided that the desired function as described herein is maintained.

In one aspect of the invention, there is provided an immunogenic peptide of 8 to 50 amino acids in length comprising at least one PASD1 epitope, wherein the epitope has the amino acids sequence of any one of SEQ ID NOs 1, 3, 5, 7, 9, 11 or 13, or a functional variant thereof. The functional variant epitope sequence varies from the ‘parent’ epitope PASD1 sequence in that one or more amino acids are, for example, either deleted, inserted, substituted or otherwise chemically modified, as explained in more detail below. A peptide of the invention may comprise more than one epitope, for example it may comprise 1, 2, 3, 4, 5, 6, or more epitopes.

‘Epitope’ refers to that part of a peptide which is capable of binding to an MHC molecule and elicit an immune response. It may be a T cell epitope.

‘PASD1 epitope’ indicates that the sequence of the epitope is derived from PASD1. Unless the context indicates otherwise, PASD1 refers to both PASD1_v1 (SEQ ID NOs 34 and 35, respectively) and PASD1_v2 (SEQ ID NO 36 and 37, respectively). “Derived from” in this context is used to indicate that the sequence of the epitope is either identical to a partial sequence of the PASD1_v1 or PASD1_v2 amino acid sequence, or that the epitope sequence represents a functional variant of such a (parent) sequence. The PASD1 epitope may be a T cell epitope. Preferably, the PASD1 epitope sequence is derived from the carboxy region of PASD1, and most preferably from the region of amino acid 468 to amino acid 639 in PASD1_v1 (SEQ ID NO. 35) or from amino acid 468 to amino acid 773 in PASD1_v2 (SEQ ID NO 37).

Thus, the invention relates to immunogenic peptides comprising a PASD1 (SEQ ID NO 35 or 37) subsequence or a functional variant thereof, which subsequence or variant effects, facilitates or contributes to the binding of the peptide to an MHC molecule. Preferably, the subsequence is a subsequence of the carboxy region of PASD1, and most preferably from the region of a.a. 468 to a.a. 639 in PASD1_v1 (SEQ ID NO. 35) and from a.a. 468 to amino acids 773 in PASD1_v2 (SEQ ID NO 37).

In one aspect of the invention there is provided a peptide of 8 to 50 amino acids in length comprising at least one T cell epitope, wherein the T cell epitope has the amino acids sequence of any one of SEQ ID NOs 1, 3, 5, 7, 9, 11 or 13 or a functional variant thereof. The functional variant epitope sequence varies from the ‘parent’ epitope PASD1 sequence in that one or more amino acids are, for example, either deleted, inserted, substituted or otherwise chemically modified, as explained in more detail below.

In one aspect of the invention there is provided an immunogenic peptide of 8 to 50 amino acids in length comprising any one of SEQ ID NOs 1, 3, 5, 7, 9, 11 or 13 or a functional variant thereof. The functional variant epitope sequence varies from the ‘parent’ epitope PASD1 sequence in that one or more amino acids are, for example, either deleted, inserted, substituted or otherwise chemically modified, as explained in more detail below.

A ‘functional variant’ in accordance with the invention, is capable to effect, facilitate or contribute to MHC binding, preferably to MHC class I binding, more preferably to HLA-A binding, more preferably to HLA-A2 binding, most preferably to HLA-A*0201, and induce a T cell specific immune response. Preferably, the T cell specific response is a HLA-A2 restricted immune response.

The ‘functional variant’ subsequence varies from the ‘parent’ PASD1 subsequence in that one or more amino acids are either deleted, inserted, substituted or otherwise chemically modified (e.g. acetylated, phosphorylated, glycosylated, or myristoylated). The variant may be 8 amino acids in length. The skilled person will appreciate, that an 8mer peptide in accordance with the invention may be obtained, for example, by deleting one amino acid of SEQ ID NOs 1, 3, 5, 7, 9, 11 or 13 or of a 9mer variant thereof, such as SEQ ID NOs 15, 17, 19, 21, 23 or 25, as long as the resulting 8mer still shows the desired properties described herein.

The variant may be a naturally occurring allelic variant as well as a synthetically produced or genetically engineered variant.

The functional variant may be generated by modifying the parent PASD1 subsequence, for example by substituting, deleting or adding one or more amino acids. Modification may occur at any position of the subsequence. With respect to a 9 amino acid subsequence, the modification may be at position 1, 2, 3, 4, 5, 6, 7, 8 or 9 of the subsequence, preferably at the amino acids that anchor the peptide to the MHC molecule. Preferably a modification may be at position positions 2 or 9. There may be one or more modifications compared to the parent subsequence. For example, there may be two or three modifications. If there are two or more modifications, the two or more modifications may be at any position of the subsequence. Preferably, there is a modification at position 2 and 9.

Modifications may be conservative modifications, i.e. the variant subsequence may be a conservatively modified variant, or non-conservative substitutions.

The skilled person will appreciate, that the term “conservative modification” or “conservative substitution” applies to both the amino acid sequence of the peptides of the invention, as well as to the nucleic acid molecules encoding them, which also form part of the invention. Preferably, nucleotide sequence changes may be made so as to minimise the difference in nucleotide sequence between the parent and the modified sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservative modification” when the alteration results in the substitution of one or more amino acids with one or more chemically similar amino acids. Conservatively modified variants typically provide similar biological activity as the unmodified polypeptide sequence from which they are derived. For example, substrate specificity, enzyme activity, or ligand/receptor binding is generally at least 60%, more preferably at least 70%, more preferably at least 80%, more preferably at least 90%, more preferably at least 95%, preferably 80-95% of the native protein for its native substrate. Conservative substitution tables providing functionally similar amino acids are well known in the art.

For example, one or more amino acid residues within the sequence can be substituted by another amino acid of a similar polarity, which acts as a functional equivalent, resulting in a silent alteration. Substitutes for an amino acid within the sequence may be selected from other members of the class to which the amino acid belongs. For example, nonpolar (hydrophobic) amino acids include alanine, leucine, isoleucine, valine, proline, phenylalanine, tryptophan and methionine. Polar neutral amino acids include glycine, serine, threonine, cysteine, tyrosine, asparagine, and glutamine. Positively charged (basic) amino acids include arginine, lysine and histidine. Negatively charged (acidic) amino acids include aspartic acid and glutamic acid. Preferably, the substitution is introducing a leucine, isoleucine, valine.

For example, a conservative modification allows substitution of one hydrophobic residue for another, or the substitution of one polar residue for another. As is well known to those skilled in the art, altering the primary structure of a polypeptide by a conservative substitution may not significantly alter the activity of that peptide because the side-chain of the amino acid which is inserted into the sequence may be able to form similar bonds and contacts as the side chain of the amino acid which has been substituted out. This is so even when the substitution is in a region which is critical in determining the peptides conformation.

With respect to nucleic acid sequences, conservatively modified variants comprise those nucleic acids that encode identical or conservatively modified variants of the amino acid sequences. Because of the degeneracy of the genetic code, a large number of functionally identical nucleic acids encode any given protein or peptide. Such nucleic acid variations are “silent variations” and represent one species of conservatively modified variation. Every nucleic acid sequence herein that encodes a protein or peptide also describes every possible silent variation of the nucleic acid. One of ordinary skill will recognize that each codon in a nucleic acid (except AUG, which is ordinarily the only codon for methionine or tga which ordinarily is the only codon which provides a stop signal for transcription) can be modified to yield a functionally identical molecule. Accordingly, each nucleic acid disclosed herein also includes each silent variation of the nucleic acid, which encodes a peptide of the present invention.

The modification may be a non-conservative modification. It may comprise substitution of one or more amino acids of one class with one or more amino acids of another class. As is well known to those skilled in the art, substitutions in regions of a peptide which are not critical in determining its conformation may not greatly affect its activity because they do not greatly alter the peptide's three dimensional structure. In regions which are critical in determining the peptides conformation or activity such changes may confer advantageous properties on the peptide. Suitable unnatural amino acids include, for example, D-amino acids, ornithine, diaminobutyric acid ornithine, norleucine ornithine, pyriylalanine, thienylalanine, naphthylalanine, phenylglycine, alpha and alpha-disubstituted amino acids, N-alkyl amino acids, lactic acid, halide derivatives of natural amino acids, such as trifluorotyrosine, p-Cl-phenylalanine, p-Br-phenylalanine, p-1-phenylalanine, L-allyl-glycine, β-alanine, I-a-amino butyric acid, L-γ-amino butyric acid, L-a-amino isobutyric acid, L-ε-amino caproic acid, 7-amino heptanoic acid, L methionine sulfone, L-norleucine, L-norvaline, p-nitro-L-phenylalanine, L-hydroxyproline, L-thioproline, methyl derivatives of phenylalanine-such as 1-methyl-Phe, pentamethyl Phe, L-Phe(4-amino), L-Tyr(methyl), L-Phe(4-isopropyl), L-Tic(1,2,3,4-tetrahydroisoquinoline-3-carboxyl acid), L-diaminopropionic acid and L-Phe(4-benzyl).

Methods for introducing modifications into an amino acid sequence or into a nucleic acid sequence are known in the art.

Modification may also be introduced into a particular amino acid or nucleotide sequence in silico, i.e. by means of bio-computer tools. The resulting sequence may then be analysed in silico for its predicted properties. Any desired peptide or nucleic acid molecule may then be artificially synthesized.

In a further aspect of the invention, there is provided a method of generating an immunogenic variant peptide, the method comprising
(i) obtaining a parent peptide, the parent peptide comprising at least one copy of a subsequence of at least 9 consecutive amino acids, wherein the subsequence is any one of SEQ ID NOs 1, 3, 5, 7, 9, 11 and 13,
(ii) modifying the subsequence of the parent peptide by substitution, deletion or insertion of one or more amino acids (thereby generating a variant peptide), and
(iii) testing the variant peptide of (ii) for immunogenicity.

In particular, the variant may be tested for its ability to bind to an MHC molecule and to induce a T cell specific immune response. Methods for testing the variant peptide for immunogenicity are known in the art. Examples of suitable techniques are discussed further below and are also set out in the examples.

Such techniques include, for example, the assessment of the binding by the peptides to T2 cells, showing stabilisation of the HLA-A2 molecule on the T2 cells surface. This can be performed at one time point or as a time course to indicate off-rates of the peptide. Further techniques include: i) mixed lymphocyte reactions in which monocyte derived-dendritic cells are loaded with peptide and the stimulation of T cells is assessed by proliferation assays (3H-thymidine), ii) cytokine secretion assays (IFN gamma secretion measured by ELISA or ELISpot assays), iii) IFN gamma production measured by intracellular cytokine assays by flow cytometry, iv) CBA bead assays to determine the array of cytokines produced following stimulation, v) quantitative measurement of the presence or expansion of specific-T cells using streptamers, tetramers or pentamers (i.e. multimers of peptide-MHC to which T cells bind if they recognise the specific peptide presented on the MHC) in flow cytometry assays or pMHC arrays and vi) purification of peptide-specific T cells using streptamers, tetramers or pentamers for further studies of cytokine secretion or CTL killing (see vi) and vii) CTL killing assays (chromium release, in vivo CTL assays or JAM assays), in which target cells may be peptide loaded or endogeneously express the antigen of interest and the response of T cells to the targets by virtue of CFSE dye or T cell proliferation or chromium release is measured.

A peptide of the invention may comprise a PASD1 epitope, the epitope consisting of any one of SEQ ID NOs 1, 3, 5, 7, 9, 11 or 13. Preferably, the epitope consists of the sequence SEQ ID NO 9 or SEQ ID NO 1.

A functional variant of said PASD1 subsequences may consist of any of SEQ ID NOs 15, 17, 19, 21, 23 of 25, preferably any of SEQ ID NOs 21, 15, 17 or 19, more preferably of SEQ ID NO 21. Thus, in one aspect the present invention provides an immunogenic peptide (of 8 to 50 amino acids in length) comprising of, essentially consisting of or consisting of any one of SEQ ID NOs 1, 3, 5, 7, 9, 11, 13, 15, 17, 19, 21, 23 or 25.

In some embodiments, the present invention provides an immunogenic peptide (of 8 to 50 amino acids in length) comprising of, essentially consisting of or consisting of any one of SEQ ID NOs 21, 9, 15, 17, 19 or 11.

In some embodiments, the present invention provides an immunogenic peptide (of 8 to 50 amino acids in length) comprising of, essentially consisting of or consisting of any one of SEQ ID NOs 21, 9, 15 or 17.

In some embodiments, the peptide may only consist of said epitope. The immunogenic peptide of the invention may only consist of a PASD1 subsequence of 9 amino acids as described herein, or a variant thereof. An immunogenic peptide in accordance with the invention may essentially consist of any one of SEQ ID NOs 1, 3, 5, 7, 9, 11, 13, 15, 17, 19, 21, 23 or 25. Any of these sequences may thus be a “parent” sequence to give rise to a variant by substituting one or more amino acids. Preferably it consists of SEQ ID NOs 21, 9, 23, 25, 1, 15, 17, or 19.

By ‘essentially consist’ it is understood that minor modifications, that do not significantly alter the function of the immunogenic peptide, are embraced.

A peptide of the invention may thus consist of any of SEQ ID NOs 1, 3, 5, 7, 9 or 11 with one or more amino acid substitutions. A peptide of the invention may consist of any of SEQ ID NOs 1, 3, 5, 7, 9 or 11 with one amino acid substitution. Preferably, it consists of SEQ ID NO 9 with one substitution. It may thus consist of SEQ ID NOs 21, 15 or 17. Preferably, the peptide of the invention consists of SEQ ID NO 1 with one substitution. It may thus consist of SEQ ID NO 23 or 25.

The substitution may replace an amino acid of a parent sequence with a leucine, isoleucine or valine residue.

An immunogenic peptide in accordance with the invention may consist of any one of SEQ ID NOs 1, 3, 5, 7, 9, 11, 13, 15, 17, 19, 21, 23 or 25. Preferably it consists of SEQ ID NOs 21, 9, 23, 25, 1, 15, 17, or 19, most preferably of SEQ ID NO 21.

In one aspect the invention provides an immunogenic peptide comprising, essentially consisting of or consisting of SEQ ID NO. 9 (P8) or a variant sequence thereof. The variant sequence comprises at least one amino acid substitution compared to SEQ ID NO. 9. For example, it may comprise 1, 2, 3, 4, 5 or more substitutions. In one aspect the invention provides an immunogenic peptide comprising, essentially consisting of or consisting of any one of SEQ ID NOs 9, 21, 15 and 17.

In one aspect the invention provides an immunogenic peptide comprising, essentially consisting of or consisting of SEQ ID NO. 1 (P4) or a variant sequence thereof. The variant sequence comprises at least one amino acid substitution compared to SEQ ID NO. 9. For example, it may comprise 1, 2, 3, 4, 5 or more substitutions. In one aspect the invention provides an immunogenic peptide comprising, essentially consisting of or consisting of any one of SEQ ID NOs 1, 23 and 25.

In one aspect the invention provides an immunogenic peptide comprising, essentially consisting of or consisting of SEQ ID NO. 11 (P9) or a variant sequence thereof. The variant sequence comprises at least one amino acid substitution compared to SEQ ID NO. 9. For example, it may comprise 1, 2, 3, 4, 5 or more substitutions. In one aspect the invention provides an immunogenic peptide comprising, essentially consisting of or consisting of any one of SEQ ID NOs 11 and 19.

The peptide may comprise one or more subsequences/epitopes as described herein, wherein the subsequences/epitopes may be the same or different subsequences/epitopes. A peptide of the invention may thus contain multiple epitopes, which may allow binding to different MHC molecules, for example to both MHC class I and II. For example, shorter peptides, such as 8-10 amino acids, which would normally bind MHC class I, could be extended to include a class II epitope, which may still encompass the class I epitope or form part of it, within, before, after or as part thereof. Epitopes could overlap. A peptide of the invention may contain a CTL epitope and a TH epitope. It may contain one or more CTL epitope(s) and/or one or more TH epitope(s). It may contain epitopes for different HLAs. It may contain one or more class I epitope(s) and/or one or more class II epitope(s). For example (but not limited to_) it may contain one or more, preferably 1 or 2 or 3 (or more) HLA-A2 epitopes and/or one or more, preferably 1 or 2 or 3 (or more) HLA-DR1 epitope and/or one or more, preferably 1 or 2 or 3 (or more) HLA-DR4 epitopes. As discussed below, the peptide may be linked to molecules or substances which enhance the immunogenicity thereof, such as (but not limited to) TLRs, for example TLR9. It may contain epitopes from different antigens.

The peptide(s) of the invention may be conjugated or fused to one or more other peptides or lipids, that may confer a desired property to the peptide, e.g. for detection or purification. For example, the peptide of the present invention can be fused to a so-called marker which enables the localization of the peptide in a cell or tissue. Suitable markers include “epitope tags” (like c-myc, hemagglutinin, FLAG-tag), biotin, digoxigenin, (strept-) avidin, Green Fluorescent Protein (GFP, and derivatives thereof), enzymes like horseradish peroxidase, alkaline phosphatase, beta-galactosidase, luciferase, beta-glucuronidase and beta-lactamase. Examples for fusion partners that allow for the purification of the peptide include HIS-tag and glutathion S transferase (GST).

It may also be useful if the peptide is fused to an immunogenic carrier or moiety, which can for example be any macromolecule that enhances the immunogenicity of the peptide. Examples of such immunogenic carriers include keyhole limpet hemocyanin (KLH), recombinant exoprotein A (rEPA), diphtheria protein CRM9 and tetanus toxin (TT).

The conjugation or fusion of the peptide to any of the modifying compounds described supra can occur by any suitable method known to the skilled artisan, either by chemical or gene technological methods. The latter requires, that a nucleic acid coding for the whole fusion construct is inserted into an expression vector and expressed as an entity.

For activating and/or inducing a T cell specific response, one of the above-described peptides may be used or they may be used in combination of two or more.

### Polyepitope Strings

In a further aspect there is provided a polyepitope string (also referred to as a polyepitope) comprising at least one of the epitopes of the invention, and comprising a further epitope. The further epitope may be an epitope according to the invention, or may be an epitope of a different antigen, i.e. not a PASD1 epitope. The further epitope may be a TAA epitope. “Polyepitope string” is a term known in the art and refers to epitopes for defined haplotypes joined together, often by amino acids, such as three alanines, or in the form of overlapping long peptides which the processing machinery can chop into defined epitopes for presentation on cell's MHC.

Polyepitope strings allow combination of epitopes that have specificity for different HLA variants (e.g. A2, A3, etc) present in a population so that with the same polyepitope one can target various HLA variants, both common and non-common. For example, HLA-A2 epitopes may be combined with epitopes specific for other HLA variants.

Polyepitope strings make it possible to deliver multiple epitopes with a range of HLA restrictions or the same HLA restrictions to prevent immune evasion by the tumour. For example, polyepitope strings of the invention may comprise multiple, preferably 2, 3, 4 or 5 (but possibly more) HLA-A2 epitopes. They may contain epitopes for differing MHC restrictions or class I and class II or minor histcompatability antigens, for example. This may overcome the issue of variation in HLA distribution amongst different populations, allowing a vaccine that can be used in a greater percentage of the population (see for example, Toes et al, 1997, PNAS 94: 14660-14665).

A string comprises at least 2 epitopes from one or more antigens. For example, there may be 2, 3, 4, 5, 6, 7, 8, 9, or more epitopes. The epitopes may include for example, CTL epitopes, and/or T-helper epitopes. In one embodiment the epitopes are preferably those presented by MHC class I molecules, in particular, HLA-2 such as HLA-A*0201 molecules.

A string may comprise multiple copies, such as 2 or more, of the same epitope, and/or different epitopes. A string may comprise multiple copies, such as 2 or more, of epitopes to the same restriction, and/or epitopes to different restrictions. (Restriction refers to the MHC molecules present on a cell such as HLA-A*0201 or HLA-A*0101 which restricts which epitopes may be presented on the groove of the available MHC molecules.) Thus a string may comprise two or more copies of an epitope of the invention.

A string may comprise only epitopes of the invention. In one embodiment the string comprises at least one other epitope in addition to (an) epitope (s) of the invention.

In some embodiments at least one additional epitope is of a TAA (tumour associated antigen). For example, suitable TAAs include members of the transmembrane 4 superfamily (TM4SF), such as human melanoma-associated antigen ME491, human and mouse leukocyte surface antigen CD37, and human lymphoblastic leukemia-associated TALLA-1 (Hotta, H. et al, (1988) Cancer Res. 48, 2955-2962; Classon, B. J. et al (1989) J. Exp. Med. 169:1497-1502; Tomlinson, M. G. et al (1996) Mol. Immun. 33:867-872; Takagi, S. et al (1995) Int. J. Cancer 61:706-715)), the PRAME antigen (Kessler et al, (2001) J. Exp. Med. 193:73-88), MAGE family antigens (Lurquin et al, J. Exp. Med 2005, 201: 249-257), high risk Human Papilloma virus (HPV) (Kast et al, (1993) J. Immunother. 14:115-20, Ressing et al, (2000) J. Immunother. 23:255-266) and the p53 tumour suppressor protein (Houbiers et al, (1993) Eur. J. Immunol. 23:2072-7). Particularly with regards to known leukaemia associated antigens (LAAs) such as, for example but not limited to, the cancer-testis antigens renal antigen-1 (RAGE-1)¹ and HAGE (named such because it has the same pattern of expression as genes of the MAGE family)²⁰ and the LAAs Wilm's Tumour-1 (WT-1)⁹, Synovial Sarcoma X breakpoint 2 interacting protein (SSX2IP)¹⁹, CA9/G250²¹, receptor for hyaluronic acid-mediated motility (RHAMM)²¹, meningioma antigen 6 (MGEA6)¹⁹, PRAME (as above and^{46,47}) and proteinase 3 (PRTN3)¹³, would make good candidates.

Other TAAs include TAAs in the following classes: cancer testis antigens (HOM-MEL-40), differentiation antigens (HOM-MEL-55), overexpressed gene products (HOM-MD-21), mutated gene products (NY-COL-2), splice variants (HOM-MD-397), gene amplification products (HOM-NSCLC-11) and cancer related autoantigens (HOM-MEL-2.4) as reviewed in Cancer Vaccines and Immunotherapy (2000) Eds Stern, Beverley and Carroll, Cambridge University Press, Cambridge. Further examples include MART-1 (Melanoma Antigen Recognised by T cells-1) MAGE-A (MAGE-A1, MAGE-A2, MAGE-A3, MAGE-A4, MAGE-A6, MAGE-A8, MAGE-A10, MAGE-A12), MAGE B (MAGE-B1-MAGE B24), MAGE-C (MAGE-C1/CT7, CT10) GAGE (GAGE-1, GAGE-8, PAGE-1, PAGE-4, XAGE-1, XAGE-3), LAGE (LAGE-1a (1S), -1b(1L), NY-ESO-1), SSX (SSX1-SSX-5), BAGE, SCP-1, PRAME (MAPE), SART-1, SART-3, CTp11, TSP50, CT9/BRDT, gp100, MART-1, TRP-1, TRP-2, MELAN-A/MART-1, Carcinoembryonic antigen (CEA), prostate-specific antigen (PSA), MUCIN (MUC-1) and Tyrosinase. In addition there are tumour viral antigens and epitopes such as those of HPV, HCV, HBV, HTLV1, EBV, Herpesvirus 8 (Little A M and Stern P L, (1999) Mol. Med. Today 5:337-342). TAAs are reviewed in Cancer Immunology (2001), Kluwer Academic Publishers, The Netherlands.

Preferably the TAAs are expressed by the same tumour type.

The polyepitope string typically includes linking sequence between the epitopes. Any suitable linking sequence of any suitable length may be used. For example, the linking sequence may be 3 amino acids in length. The linking sequence typically comprises spacer sequence, preferably polyalanine sequence such as that in Toes et al, 1997, PNAS 94:14660-14665. In general the linking sequence comprises at least one proteolytic site between each pair of epitopes and allows exact C-terminal excision of the epitope by proteosomal cleavage.

Preferably the linker does not include sequence which precludes, e.g. by secondary structure, direct antigen processing by the proteasome. Preferably the sites are also cleavable by alternative cellular enzymes in a host cell. This will allow processing of the epitopes by the cell, for example, display of an epitope in the string on the cell surface bound to an MHC molecule.

Polyepitope strings may be prepared using methods known in the art (see for example Toes et al, 1997, PNAS 94:14660-14665).

Peptides or polyepitope strings of the invention may be in (substantially) isolated form. A peptide or string may be mixed with carriers or diluents which will not interfere with the intended purpose of the peptide/string and will still be regarded as substantially isolated. A peptide or string may also be in substantially purified form, in which case it will generally comprise the peptide/string in a preparation in which more than 30%, more than 32%, more than 35%, more than 50%, more than 60%, more than 70%, more than 80%, 90%, 95% or 99% by weight, such as 100% of the peptide/string in the preparation is a peptide/string of the invention.

Peptides and polyepitope strings may be provided in association with molecules or substances which enhance the immunogenicity thereof. For example, a substance may facilitate or enhance cell entry or penetration by the peptide/string, cellular processing or transport of epitope to the cell surface. Examples of suitable molecules or substances include adjuvants (described herein), transporter peptides such as TAP, lipids and other cell targeting molecules, in particular substances docking onto dendritic cells, with or without additional dendritic cell activating ability such as receptors for heat shock proteins (scavenger receptors), Fc receptors, C-type lectins and TLR ligands, such as TLR9.

‘In association’ includes covalent bonding, non-covalent bonding (e.g. electrostatic) and other interactions. For example the peptides or strings may be provided fused to one or more of the molecules or substances.

### Nucleic Acids

In a further aspect of the invention, there is provided a nucleic acid encoding a peptide of the invention.

Nucleic acid as used herein may include cDNA, RNA, genomic DNA (single or double stranded) and modified nucleic acids or nucleic acid analogues. Where a nucleic acid of the invention is referred to herein, the complement of that nucleic acid is also embraced by the invention. The complement in each case is the same length as the reference, but is 100% complementary thereto whereby each nucleotide is capable of base pairing with its counterpart.

A nucleic acid of the invention may be obtained by any suitable means. For example it may be (i) obtained by amplification in vitro, for example by PCR; or (ii) recombinantly produced by cloning; or (iii) purified from a natural source; or (iv) artificially synthesized, such as by chemical synthesis.

In some embodiments of the invention, a nucleic acid encoding a peptide of the invention may comprise one or more of the nucleic acid sequences of SEQ ID NOs 2, 4, 6, 8, 10, 12, 14, 16, 18, 20, 22, 24 or 26, preferably one or more of 10, 22, 2, 24, 26, 16 or 18.

In some embodiments of the invention there is provided a nucleic acid consisting essentially of any one of SEQ ID NOs 2, 4, 6, 8, 10, 12, 14, 16, 18, 20, 22, 24 or 26, preferably any one of 10, 22, 2, 24, 26, 16 or 18.

“Consisting essentially of” indicates that minor modifications, that do no result in a substantial change of the immunogenicity of the encoded protein are embraced. The nucleic acid may thus consist of any one of the nucleic acid sequences of SEQ ID NOs 2, 4, 6, 8, 10, 12, 14, 16, 18, 20, 22, 24 or 26 with one or more nucleotide substitutions.

In some embodiments of the invention there is provided a nucleic acid consisting of any one of SEQ ID NOs 2, 4, 6, 8, 10, 12, 14, 16, 18, 20, 22, 24 or 26, preferably any one of 10, 22, 2, 24, 26, 16 or 18.

Moreover, a nucleic acid that hybridizes to the above-described nucleic acid under stringent conditions is included in the scope of the present invention. In the case where the nucleic acid is a DNA molecule, “a DNA molecule that hybridizes to a DNA molecule under stringent conditions” can be obtained, for example, by the method described in “Molecular Cloning: A Laboratory Manual (Cold Spring Harbor Laboratory, 1989.)” “To hybridize under stringent conditions” herein means that a positive hybridizing signal is still observed even under conditions, for example, where hybridization is carried out in a solution containing 6×SSC, 0.5% SDS, and 50% formamide at 42° C., and then, washing is carried out in a solution containing 0.1×SSC and 0.5% SDS at 68° C.

A nucleic acid capable of hybridising to nucleic acids of the invention will generally exhibit a homology of at least 60%, preferably at least 70%, preferably at least 80%, preferably at least 85%, preferably at least 90%, preferably at least 95%, 96%, 97%, 98% or 99% to the nucleic acids of the invention.

All of the above-described nucleic acids provide genetic information useful for producing a polyepitope or a peptide according to the present invention or can be also utilized as a reagent and a standard of a nucleic acid. In a further aspect of the invention, there is provided a particle coated with a peptide or a nucleic acid of the present invention.

Nucleic acids and vectors may be delivered using a particle mediated method. Typically the nucleic acid is immobilised on solid particles and delivered by means of a gene gun or particle mediated delivery device into tissue or cells. Suitable methods are known in the art. Thus in one aspect the invention relates to solid phase particles coated with a polynucleotide or vector of the invention. Typically the particles are gold particles. The invention also relates to a gene gun or particle acceleration device, and a cartridge for such a device, loaded with the particles.

Particles could also be incorporated into DNA to aid tracking/detection of individual diseased cells i.e. antigen detection, involving giving certain fluorescent or metal nanomolecules an affinity for a specific antigen or protein, to aid targeting i.e. of chemotherapy, therapy, immunotherapy, targeted therapy, antibody therapy, growth inhibitors can be attached to nanoparticles which could then target an antigen/epitope of interest (ref: Kawasaki, Ernest S., and Audrey Player. “Nanotechnology, nanomedicine, and the development of new, effective therapies for cancer.” Nanomedicine: Nanotechnology, Biology and Medicine 1 (2005): 101-109).

### Vectors and Cells

For the introduction of a peptide of the invention, respectively the nucleic acid encoding it, into a suitable host cell and its expression it can be advantageous if the nucleic acid is integrated in an expression vector. Cloning techniques to introduce a nucleic acid into a suitable expression vector for subsequent transformation of a cell and subsequent selection of the transformed cell are known in the art (see for example Sambrook et al. (1989), Molecular cloning: A laboratory Manual, Cold Spring Harbour Laboratory).

In a further aspect there is thus provided a vector, preferably an expression vector, comprising a nucleic acid encoding a peptide of the invention. Suitable vectors are known in the art.

The expression vector is preferably a eukaryotic expression vector, or a retroviral, lentiviral, adenoviral or adenoviral associated vector, a plasmid, bacteriophage, or any other vector typically used in the biotechnology field. The vectors may contain one or more selection markers, such as an antibiotic resistance marker, for example. The nucleic acid encoding the peptide of the invention may be operatively linked to one or more regulatory elements which modulate the transcription and the synthesis of a translatable mRNA in pro- or eukaryotic cells. Such regulatory elements may be promoters, enhancers or transcription termination signals, but can also comprise introns or similar elements, for example those which promote or contribute to the stability and the amplification of the vector, the selection for successful delivery and/or the integration into the host's genome, like regions that promote homologous recombination at a desired site in the genome. For therapeutic purposes, the use of retroviral vectors has been proven to be most appropriate to deliver a desired nucleic acid into a target cell, although for primary leukaemia cells which are not dividing, lentiviruses often work while retroviruses predominantly do not.

Nucleic acid molecules of the invention may be inserted into the vectors described herein in a sense orientation, or in an anti-sense orientation in order to provide for the production of anti-sense RNA.

The vectors described herein may be transformed into a host cell to allow expression of a peptide in accordance with the invention. The cell may be part of a tissue or an organism.

The vector may be delivered to a cell as naked DNA.

The expression vector may be a plasmid, in particular a pDOM plasmid. As described above, DNA fusion vaccines were initially developed to treat B-cell malignancies². Fusion of the microbial sequence, Fragment C (FrC) from tetanus toxin, to idiotypic tumour antigen, was shown to provide the T cell help required to induce humoral³ and CD4⁺ T cell responses in pre-clinical models⁴. For induction of CD4⁺ T-cell responses, the vaccine design was modified by reducing the fragment C (FrC) sequence to a single domain (DOM), which decreased the potential for peptide competition but retained the promiscuous MHC class II peptide p308. An epitope-specific sequence was then inserted at the C terminus of DOM to provide the CD8+ specific target. In multiple models^{5,8,9,11}, this p.DOM-epitope design was able to induce high levels of epitope-specific CD8⁺ T cells.

Thus, in some embodiments there are provided pDOM plasmids carrying nucleic acids of the invention. The DOM1 first domain of tetanus toxin induces CD4+ help that aids good CD8+ responses. Other plasmid backbones, for example the pcDNA plasmids from Invitrogen, could be used and other CD4+ stimulators, for example class II epitopes from the antigen of interest, from Flu, or from CMV, or other viral antigens to which humans are immunized during childhood such as BCG could be used.

In one aspect of the invention there is provided a vector, preferably an expression vector, comprising a nucleic acid encoding a peptide of the invention. The pDOM plasmid comprises CpG sites and a gene encoding the first domain of FrC of tetanus toxin (DOM, TT865-1120) with a leader sequence derived from the VH of the IgM of the BCL1 tumour at the N-terminus (4,5,7,8,10,28-31). This first domain of tetanus toxin is used to provide tumour specific antibody, CD4+ and CD8+ responses when linked to a tumour associated nucleotide sequence, encoding the peptide of interest. This format allows the appropriate processing and presentation of the peptide.

### Transformants

The vector in which the above-described nucleic acid has been inserted can be used to obtain a transformant by transforming a well-known host such as Escherichia coli, yeast, Bacillus subtillis, leishmania, an insect cell, or a mammalian cell therewith by well-known methods. In the case of carrying out the transformation, a more preferable system is exemplified by the method for integrating the gene in the chromosome, in view of achieving stability of the gene. However, an autonomous replication system using a plasmid can be conveniently used. Introduction of the DNA vector into the host cell can be carried out by standard methods such that described in “Molecular Cloning: A Laboratory Manual” (ed. by Sambrook et al., Cold Spring Harbor Laboratory Press, Cold Spring Harbor, N.Y., 1989.) In particular, calcium phosphate transfection, DEAE-dextran-mediated transfection, microinjection, cation lipid-mediated transfection, electroporation, transduction, scrape loading, ballistic introduction and infection can be employed.

In a further aspect there is provided a host cell transformed or transfected with an expression vector of the invention.

In a further aspect there is provided a cell pulsed, transformed or transfected with a peptide, a polyepitope string, a nucleic acid, a vector or a particle of the invention, respectively, or a combination thereof.

In some embodiments the cell is capable of presenting the peptide of the invention on the cell surface. The cell may be an antigen-presenting cell.

The cell comprising the peptide of the invention or the nucleic acid encoding it may be a professional antigen-presenting cell such as a B cell, a macrophage or a dendritic cell, or any other cell within which the peptide can be loaded onto the HLA molecule and transported to the cell surface and presented as an antigen in order to induce the described immune response.

The cell comprising the peptide of the invention may be a T2 cell.

In particular, dendritic cells have been proven to be especially useful as vaccination “vehicles”. Dendritic cells which are located in nearly all tissue types of the body incorporate a compound like peptide and migrate together with the lymph stream to the lymph node where they encounter with precursors of antigen-specific cytotoxic T cells. For the purposes of the present invention as well as for therapeutic purposes in general, dendritic cells can be generated and cultured in vitro by cultivating adherent cells rich in monocytes or bead purified CD14+ cells in the presence of cytokines, including but not limited to, Interleukin-4 (IL-4), interleukin-7 and Granulocyte Macrophage Colony Stimulating Factor (GM-CSF), TNFα, IL-6, IL-1β; or combination thereof. Further, postaglandin (PGE2) may be present. Alternatively, dendritic cells can be generated from CD34+ haematopoietic stem cells of the periphery blood. By systematic application of growth factors, like e.g. Flt3 ligand, dendritic cells can also be expanded in the blood in vivo by several orders of magnitude.

Isolated dendritic or other professional antigen-presenting cells can be loaded (“pulsed”) with a peptide of the invention or the nucleic acid encoding it in order to enable the presentation of the peptide on the surface of these cells.

Thus, in a further aspect of the invention there is provided an antigen presenting cell (APC) pulsed, transformed or transfected with a peptide of the invention.

The APC may be, for example, a macrophage, a B cell or a dendritic cell.

APCs can be categorized into two categories: professional or non-professional.

Most cells in the body can present antigen to CD8⁺ T cells via MHC class I molecules and thus act as “APCs”. However the term is often limited to those specialized cells that can prime T cells (i.e. activate a T cell that has not been exposed to antigen, termed a naive T cell). Generally, these cells express MHC class II as well as MHC class I molecules, and can stimulate CD4⁺ (“helper”) cells as well as CD8⁺ (“cytotoxic”) T cells.

Those that express MHC class II molecules are often called professional antigen-presenting cells.

### Professional APCs

These professional APCs very efficiently internalize antigen, either by phagocytosis or by receptor-mediated endocytosis, proteolyse in the lumen of the ER and then display a fragment of the antigen, bound to a MHC class II molecule, on the cell surface. The T cell recognizes and interacts with the antigen-MHC class II molecule complex on the surface of the antigen-presenting cell. An additional co-stimulatory signal is then produced by the antigen-presenting cell, leading to activation of the T cell.

There are three main types of professional antigen-presenting cell:
Dendritic cells
Macrophages
B-cells

### Non-Professional

A non-professional APC does not constitutively express the Major histocompatibility complex proteins required for interaction with naive T cells; these are expressed only upon stimulation of the non-professional APC by certain cytokines such as IFN-γ. Non-professional APCs include:
Fibroblasts (skin)
Thymic epithelial cells
Thyroid epithelial cells
Glial cells (brain)
Pancreatic beta cells
Vascular endothelial cells

Using an expression vector for transduction of the above-described transformant, a peptide of the present invention can be provided. A transformant, transformed with an expression vector comprising the above-described nucleic acid, is cultured under culture conditions suitable for each host. Culturing may be conducted by using indicators, such as a function of the peptide of the present invention that is expressed by the transformant, for example the activity to induce and/or activate CTL, or the peptide or the amount of the peptide produced in the host or outside of the host. Subculturing or batch culturing may be also carried out using an amount of the transformant in the culture as an indicator.

A peptide or polyepitope string according to the present invention can be produced by a general method known in peptide chemistry. For example, “Peptide Synthesis (Maruzen) 1975” and “Peptide Synthesis, Interscience, New York, 1996” are exemplified. However, any widely known method can be used.

A peptide or polyepitope string according to the present invention can be purified and collected by a method, such as a gel filtration chromatography, an ion column chromatography, an affinity chromatography, and the like, in combination, or by fractionation means on the basis of a difference insolubility using ammonium sulfate, alcohol, and the like, using for example, a CTL-activating ability of the polyepitope string or the peptide as an indicator. More preferably used is a method, wherein the peptides are specifically adsorbed and collected by using antibodies (polyclonal or monoclonal) antibodies, which are prepared against the peptides based on the information of their amino acid sequences.

### Antibodies

An antibody according to the present invention may be prepared by using the above-described peptides, or a fragment thereof that is composed of at least 5, more preferably at least 8 to amino acids, as an antigen. Thus, in one aspect the invention provides the use of a peptide as described herein in the production of an antibody against said peptide. Antibodies may be raised against a peptide of the invention, or against a peptide of the invention bound to MHC. Thus, in a further aspect the invention provides an antibody against a peptide of the invention. Preferably, the antibody specifically binds a peptide of the invention. In order to obtain antibodies specific to the peptide, a region consisting of the amino acid sequence intrinsic to the above-described peptide is desirably used. The amino acid sequence is not necessarily homologous to the amino acid sequence of the peptide, but is preferably a site exposed to outside of a stereo-structure of the peptide. In such a case, it is sufficient that the amino acid sequence of the exposed site is consecutive in the exposed site, even if it may be discrete in its primary structure. The antibody is not limited as long as it binds or recognizes the peptide immunologically. The presence or absence of the binding or the recognition can be determined by a well-known antigen-antibody binding reaction.

Any suitable method for antibody production may be employed. For example, the antibody may be obtained by administration of the peptide according to the present invention to an animal in the presence or absence of an adjuvant with or without linking such to a carrier so as to induce humoral immunity and/or cell-mediated immunity. Any suitable carrier may be used. For example, cellulose, a polymerized amino acid, albumin, and the like are exemplified, but not limited thereto. As an animal used for immunization, a mouse, rat, rabbit, goat, horse, and so on, is preferably used. Alternatively the DNA vaccine containing fragment C linked to the peptide of interest or the pDOM.epitope vaccine as it is may be used to generate antibodies against the epitope of interest in mammals.

The antibody of the invention may be a polyclonal or a monoclonal antibody. A polyclonal antibody can be obtained from serum of an animal such treated by any suitable method known in the art for collecting antibodies. A preferable method is, for example, immunoaffinity chromatography.

A monoclonal antibody can be produced by collecting antibody-producing cells (for example, a lymphocyte derived from a spleen or a lymph node) from the animal subjected to the above-described immunological means, followed by introducing a well-known transformation with a permanently proliferating cell (for example, myeloma strain such as P3/X63-Ag8 cells.) For example, the antibody-producing cells are fused with the permanently proliferating cells by a well-known method to prepare hybridomas. Then, the hybridomas are subjected to cloning, followed by selecting ones producing the antibody that recognizes specifically the above-described peptide to collect the antibody from a culture solution of the hybridoma.

A polyclonal or monoclonal antibody thus obtained, which recognizes and binds to a peptide of the invention, can be utilized as an antibody for purification, a reagent, a labeling marker and so on.

### T Cells, T Cell Lines and T Cell Receptors

In a further aspect there is provided a T cell, preferably an isolated T cell, specific for a peptide of the invention. The T cell may be a CTL or a TH cell.

In some embodiments, the invention provides an isolated T cell produced by stimulating peripheral blood mononuclear cells (PBMCs) with an epitope or peptide or polyepitope string as described herein. The isolated T cell may be a CTL or T_{H} cell.

In some embodiments of the invention, a peptide, a nucleic acid or a cell may be isolated.

The term ‘isolated’ is used to indicate that a cell, a peptide or a nucleic acid is separated from its native environment or the system where it has been produced. Isolated peptides and nucleic acids may be substantially pure, i.e. essentially free of other substances with which they may be found in nature or production systems.

Adoptively transferred cells could be sought from HLA-matched or partially matched unrelated or related donors. These disease-free/well individuals could be immunised and their T cells adoptively transferred to the sick recipient. TCR can be modified or cloned from responsive T cells and placed into T cells from the recipient conveying responsiveness to the LAA (reviewed in³²). Alternatively CD8 T cells for the invention can be purified by pMHC multimers (for example pentamers, tetramers, streptamers) and expanded ex vivo and returned to the patient for adoptive therapy treatment of their malignancy. This boosting of CTL numbers can help the patient overcome T cell tolerance to the tumour.

In a further aspect of the invention, there is provided a T cell line which specifically recognises an epitope or a peptide of the invention. Methods of generating and maintaining cell lines are known in the art.

In a further aspect of the invention, there is provided an agent which is capable of specifically binding an epitope or peptide of the invention. The agent may be an isolated T cell receptor or an antibody.

TCRs specific for the epitopes described herein can find utility both in therapy as well as diagnostic tools. For example, they may be used for targeted delivery of therapeutics.

T cells as described herein may be purified, for example by reversible purification, and then expanded and used in adoptive transfer therapy, as discussed in more detail below. In this scenario, peptide/MHC I-Strep tag is attached to fluorescent (PE or APC) Strep-Tactin oligomers prior to incubation with T cells. Streptamers bind with high affinity and selectivity to antigen-specific T-cells and these can be isolated by FACs or using magnetic beads. By the addition of low doses of biotin the T cells can be released, fully viable and phenotypically and functionally indistinguishable from non-treated cells.

In a further aspect of the invention, there is provided a monomeric, tetrameric or pentameric complex comprising a multivalent MHC molecule, and an epitope or peptide or polyepitope string of the invention. Such complexes of peptide-MHC, stabilised by their multimeric nature, may be used for the quantification of T cell numbers to test for T cell activation, in addition to purifying T cells, reversibly or non-reversibly using tetramers or streptamers.

### Testing for Immunogenicity

Peptides, nucleic acids, transformed cells or antibodies as described herein, may provide means for testing whether a particular peptide can induce a T cell response, which leads to a specific T cell expansion.

However, any suitable testing method may be employed. The method may be an in vitro or in vivo method.

For example, as shown in the Examples herein, one may employ a system in which the activation of CTL by the antigen-presenting cells that are pulsed with a peptide of the invention is measured on the basis of the amount of IFNγ production from CTL. Addition of a test substance to the system allows one to select the substance inducing and/or activating CTL and the substance enhancing the induction and/or the activation.

### Transgenics

In a further aspect of the invention, there is provided a transgenic cell, tissue or organism comprising a transgene capable of expressing a peptide according to the invention.

The term “transgene capable of expressing” as used herein encompasses any suitable nucleic acid sequence which leads to expression of a peptide of the invention, or a peptide having the same function and/or activity as the peptides of the invention. The transgene may include, for example, genomic nucleic acid isolated from human cells or synthetic nucleic acid, including DNA integrated into the genome or in an extrachromosomal state. Preferably, the transgene comprises the nucleic acid sequence encoding the peptide according to the invention as described herein, or a functional fragment of said nucleic acid. A functional fragment of said nucleic acid should be taken to mean a fragment of the gene comprising said nucleic acid coding for the peptides according to the invention or a functional equivalent, derivative or a non-functional derivative such as a dominant negative mutant of said peptides. Transgenic non-human organisms are being utilised as model systems for studying both normal and disease cell processes.

In general, to create such transgenic animals an exogenous gene with or without a mutation is transferred to the animal host system and the phenotype resulting from the transferred gene is observed. Other genetic manipulations can be undertaken in the vector or host system to improve the gene expression leading to the observed phenotype (phenotypic expression). The gene may be transferred on a vector under the control of different inducible or constitutive promoters, may be over expressed or the endogenous homologous gene may be rendered unexpressible, and the like (WO 92/11358). The vector may be introduced by transfection or other suitable techniques such as electroporation, for example, in embryonic stem cells. The cells that have the exogenous DNA incorporated into their genome, for example, by homologous recombination, may subsequently be injected into blastocytes for generation of the transgenic animals with the desired phenotype. Successfully transformed cells containing the vector may be identified by well known techniques such as lysing the cells and examining the DNA, by, for example, Southern blotting or using the polymerase chain reaction. Knock-out organisms may be generated to further investigate the role of the peptides of the invention in vivo. By “knock-out” it is meant an animal which has its endogenous gene knocked out or inactivated. Typically, homologous recombination is used to insert a selectable gene into an essential exon of the gene of interest. Furthermore, the gene of interest can be knocked out in favour of a homologous exogenous gene to investigate the role of the exogenous gene (Robbins, J., GENE TARGETING. The Precise Manipulation of the Mammalian Genome Res. 1993, J.W.; 73; 3-9). Transgenic animals, such as mice or Drosophila or the like, may therefore be used to over or under express the peptide according to the invention to further investigate their role in vivo and in the progression or treatment of diseases, such as cancer.

### Pharmaceutical Compositions

In a further aspect, the present invention provides a pharmaceutical composition comprising a peptide of the present invention and/or a polyepitope string of the present invention and/or a nucleic acid of the present invention and/or an expression vector of the present invention and/or a particle of the present invention and/or a cell of the present invention and/or a T cell of the present invention and/or an agent of the present invention and/or a complex of the present invention, and a pharmaceutically acceptable carrier or diluent. Thus, the present invention provides a pharmaceutical composition comprising one or more peptides of the present invention and/or one or more polyepitope strings of the present invention and/or one or more nucleic acids of the present invention and/or one or more expression vectors of the present invention and/or one or more particles of the present invention and/or one or more cells of the present invention and/or one or more T cells of the present invention and/or one or more agents of the present invention and/or one or more complexes of the present invention, and a pharmaceutically acceptable carrier or diluent. If the pharmaceutical composition comprises multiple peptides and/or multiple polyepitope strings and/or multiple nucleic acids and/or multiple expression vectors and/or multiple particles and/or multiple cells and/or multiple T cells and/or multiple agents and/or multiple complexes, the peptides and/or polyepitope strings and/or nucleic acids and/or expression vectors and/or particles and/or cells and/or T cells and/or agents and/or complexes may relate to the same epitope or different epitopes; they may relate to the same antigens or different antigens.

Also provided is the use of said compositions in methods of immunotherapy for treatment or prophylaxis of a human or animal subject. Various forms of immunotherapy are known in the art, such as for example (but not limited to): (i) non-viral delivery, (ii) viral delivery, (iii) PASD1-stimulated DC infusion, (iv) adoptive therapy either in the form of purified and expanded Pa14-specific T cells, TCR gene therapy and/or PASD1-stimulated donor lymphocyte infusion, (v) DNA based vaccination, such as for example (but not limited to) the pDOM technology. The products of the present invention may be used in any form of immunotherapy. Immunotherapies such as the ones mentioned above are known in the art. For example, a review of some of these immunotherapies include Guinn, B. A., Mohamedali, A., Thomas, N. S. B. & Mills, K. I. (2007) Immunotherapy of myeloid leukaemias. Cancer Immunology, Immunotherapy, 56, 943-957; Rice J, Ottensmeier C H, Stevenson F K. DNA vaccines: precision tools for activating effective immunity against cancer. Nat Rev Cancer. 2008 February; 8(2):108-20; Collins, S. A., Guinn, B. A., Harrison, P. T., Scallan, M. F., O'Sullivan, G. C. & Tangney, M. (2008) Viral Vectors in Cancer Immunotherapy: Which Vector for Which Strategy? Current Gene Therapy, 8, 66-76. Thomas S, Hart D P, Xue S A, Cesco-Gaspere M, Stauss H J. T-cell receptor gene therapy for cancer: the progress to date and future objectives. Expert Opin Biol Ther. 2007 August; 7(8):1207-18; Rosenberg S A, Restifo N P, Yang J C, Morgan R A, Dudley M E. Adoptive cell transfer: a clinical path to effective cancer immunotherapy. Nat Rev Cancer. 2008 April; 8(4):299-308.

For example, the peptides described herein may be used in a T cell based adoptive immunotherapy (ACT). In this case the treatment may include, for example, any one of the following steps:
1) obtaining from a subject a cell population containing or capable of producing CTLs and/or TH cells;
2) contacting the cell population with one or more peptide(s) of the invention;
3) screening the cell population for (and optionally isolating) CTLs and/or Th cells specific for said one or more peptide(s) and
4) administering the cell population or isolated CTLs and/or Th cells to a subject in need thereof.

The subject may be suffering or being suspected or at risk of suffering from cancer. The subject in step 1) and the subject in step 4) may be the same subject (autologous) or may be a different subject (allogeneic).

Cloning of TCR genes from the CTLs and/or Th cells with specificity for the peptides represents another therapeutic approach. This may include, for example, any one of the following steps:
1. obtaining from a subject a cell population containing or capable of producing CTLs and/or TH cells;
2. contacting the cell population with one or more peptide(s) of the invention
3. screening the cell population for (and optionally isolating) CTLs and/or Th cells specific for said one or more peptide(s);
4. cloning the TCR gene from the CTLs and/or Th cells specific for the peptide; and
5. transducing the TCR gene into cells from the patient or cells from a healthy donor; and
6. administering the transduced cells to a subject.

The subject in step 1 may be a healthy individual or an individual suffering or being suspected or at risk of suffering from cancer. The subject in step 6 may be suffering or being suspected or at risk of suffering from cancer.

The pharmaceutical composition may further comprise a soluble immunostimulant.

Pharmaceutically acceptable carriers or diluents include those used in formulations suitable for oral, rectal, nasal, topical (including buccal and sublingual), inter-nodal, vaginal or parenteral (including subcutaneous, inter/intra-peritoneal, intramuscular, intravenous, intradermal, intrathecal and epidural) administration. The formulations may conveniently be presented in unit dosage form and may be prepared by any of the methods well known in the art of pharmacy.

The products and compositions of the invention may be administered to a subject to treat, prevent or alleviate a disease, including the delay of relapse. Said diseases may be any disease amenable to the treatment with the compositions and products of the invention, for example a malignancy such as cancer, and in particular a haematologically derived malignancy such as the myeloid leukaemias including but not limited to acute myeloid leukaemia (AML), chronic leukaemia (CML) and myelodysplasic syndrome (MDS) for example. Treatment of a subject with products and compositions of the invention may be combined with other treatments. Such additional treatments may comprise radiotherapy, chemotherapy and additional immunotherapy, and may be designed for simultaneous, separate or sequential use in treatment.

### Vaccines

In a further aspect, the invention provides a vaccine comprising a peptide and/or a polyepitope string and/or a nucleic acid and/or an expression vector and/or a particle and/or a cell and/or a T cell and/or an agent and/or a complex and/or a pharmaceutical composition of the present invention, respectively, and optionally further comprising an adjuvant.

In a further aspect, the invention provides a peptide, polyepitope string, a nucleic acid, an expression vector, a particle, a cell, a T cell, an agent, a complex or a pharmaceutical composition according to the invention, respectively, for use as a vaccine. They may be used for prophylactic or therapeutic vaccination.

The vaccine of the invention may further comprise an additional TAA peptide, i.e. another peptide/epitope from PASD1 or from an antigen other than PASD1. The vaccine may comprise one or more peptides and/or one or more polyepitope strings and/or one or more nucleic acids and/or one or more expression vectors and/or one or more particles and/or one or more cells and/or one or more T cells and/or one or more agents and/or one or more complexes and/or one or more pharmaceutical compositions of the present invention, respectively, and optionally further comprising an adjuvant.

The products or pharmaceutical compositions described herein stimulate an immune response leading to the production of immune molecules. The invention comprises vaccines sufficient to reduce the number, severity and/or duration of symptoms.

DNA fusion vaccines were initially developed to treat B-cell malignancies². Fusion of the microbial sequence, Fragment C (FrC) from tetanus toxin, to idiotypic tumour antigen, was shown to provide the T cell help required to induce humoral³ and CD4⁺ T cell responses in pre-clinical models⁴. The LIFTT trial (GTAC 029A), a phase I/II dose escalation study, used individual idiotypic DNA fusion vaccines to treat patients with follicular lymphoma. The vaccine was safe and 14/18 patients showed an antibody and/or CD4⁺ T-cell responses against the FrC portion of the fusion gene. Encouragingly, 6/16 showed responses to the tumour-specific idiotypic antigen (manuscript in preparation). (The technique has been exemplified in McCarthy H, Ottensmeier C H, Hamblin T J, Stevenson F K. Anti-idiotype vaccines. Br J Haematol. 2003 December; 123:770-81, Zhu D, Rice J, Savelyeva N, Stevenson F K. DNA fusion vaccines against B-cell tumors. Trends Mol Med. 2001; 7:566-72, Stevenson F K, Zhu D, King C A, Ashworth L J, Kumar S, Thompsett A, Hawkins R E. A genetic approach to idiotypic vaccination for B cell lymphoma. Ann N Y Acad. Sci. 1995; 772:212-26 and Stevenson F K, Zhu D, King C A, Ashworth L J, Kumar S, Hawkins R E. Idiotypic DNA vaccines against B-cell lymphoma. Immunol Rev. 1995; 145:211-28). However, the levels of response were relatively low and improvements were sought. An important development has been electroporation (EP), which dramatically increased DNA vaccine performance in mice⁵ and rhesus macaques⁶ and this has been included in a current pDOM.PSMA27 clinical trial, with evidence for amplification of antibody and CD4⁺ T-cell responses'.

For induction of CD8⁺ T-cell responses, the vaccine design was modified by reducing the fragment C (FrC) sequence to a single domain (DOM), which decreased the potential for peptide competition but retained the MHC class II-restricted peptide p30⁸. An epitope-specific sequence was then inserted at the C terminus of FrC to aid processing/presentation. In multiple models^{5,7,9}, this p.DOM-epitope design was able to induce high levels of epitope-specific CD8⁺ T cells. Importantly, provision of high levels of T-cell enables induction of immune responses in tolerant settings^{10,11}. Clinical trials using this design are ongoing in prostate cancer (pDOM.PSMA27 clinical trial mentioned above) and CEA-expressing malignancies. For patients with relapsed prostate cancer, a p.DOM-epitope design incorporating a peptide sequence from PSMA (pDOM.PSMA27) has induced high levels of epitope-specific IFNγ-producing CD8⁺ T cell responses in 65% (8/12) patients to date⁷. Responses are robust and persist over several months so far. The effect of EP on the induction of CD8⁺ T-cell responses is still being evaluated⁴⁴.

In addition to products or pharmaceutical compositions as described herein, a vaccine may include salts, buffers, adjuvants and other substances, or excipients which may be desirable for improving its efficacy. The latter can be administered before, after or simultaneously with the administration of the products or pharmaceutical composition of the invention. Examples of suitable vaccine components as well as a general guidance with regard to methods for preparing effective compositions may be found in standard texts such as Remington's Pharmaceutical Sciences (Osol, A, ed., Mack Publishing Co., (1990)). In all cases, the product or composition as described herein should be present in an effective amount, i.e. an amount that produces the desired effect. Other components of the vaccine should be physiologically acceptable. The vaccine of the present invention may be administered by either single or multiple dosages of an effective amount of product or composition.

The vaccine is generally administered in effective amounts, i.e. amounts which are sufficient to induce the desired immune response.

Vaccines may be administered to subjects by any route known in the art, including parenteral routes (e.g. injection), inhalation, topical or by oral administration. Suitable methods include, for example, intramuscular, intravenous, or subcutaneous injection, or intradermal, intranodal, intraperitoneal or intranasal administration. Suitable carriers that may be used in preparations for injection include sterile aqueous (e.g., physiological saline) or non-aqueous solutions and suspensions such as propylene glycol, polyethylene glycol, vegetable oils such as olive oil, and injectable organic esters such as ethyl oleate. Treatment and dosing strategies may be developed using guidance provided by standard reference works (see e.g. N. Engl. J. Med. 345 (16):1177-83 (2001) for treatment of children, and Arch. Intern. Med 154(22):2545-57 (1994) for adult treatments; see Arch. Intern. Med 28, 154(4):373-7 (1994) for a review of clinical trials.

Vaccines may comprise naked nucleotide sequences or may be in combination with cationic lipids, polymers or targeting systems. Suitable methods for delivering naked DNA in vivo and ex vivo are known in the art. Nucleic acids can be delivered by injection intradermally, subcutaneously or intra muscularly. Alternatively a nucleic acid can be delivered across the skin using a nucleic acid delivery device such as particle mediated gene delivery. More recently electroporation ^{5,7,9}techniques have also been explored for the delivery of DNA vaccines and have demonstrated great improvement in DNA uptake. The nucleic acid may be administered topically to the skin or to mucosal surfaces for example by intranasal, oral, intravaginal or intrarectal administration.

Vaccines may be administered to a subject to treat a disease after symptoms have appeared. In these cases, it will be advantageous to initiate treatment as soon after the onset of symptoms as possible and, depending on the circumstances, to combine vaccine administration with other treatments, e.g. anti-cancer treatments such as chemotherapy or radiotherapy. Or vaccines may be administered after standard treatments such as chemotherapy and radiotherapy when tumour loads are minimal and the immune system has started to recover from conventional treatment. For example, it may be administered several months after the completion of conventional treatment and when minimal residual disease has been achieved. Different vaccine compositions could be administered in combination. Administration of other treatments could be separate, simultaneous or subsequent to treatment with vaccines or pharmaceutical compositions of the invention. Vaccines may be administration at first remission of a disease following treatment with other agents in order to maintain response by killing residual tumour cells and prevent relapse.

Different vaccine compositions could be administered in combination with each other. Administration of other treatments could be separate, simultaneous or subsequent to treatment with the vaccines or pharmaceutical compositions of the present invention.

A peptide, a polyepitope string, a nucleic acid, an expression vector, a particle, a cell, a T cell, an agent or a pharmaceutical composition according to the present invention, respectively, of the present invention may find utility as an adjuvant. An adjuvant is a substance capable of enhancing and/or extending the duration of the protective immune responses induced by antigens against a target. Antigens identified by the SEREX technology have been shown to be useful as adjuvants to boost the immune response to other tumour antigens (Nishikawa et al, 2001, PNAS USA 98:14571-14576).

### Medical Uses

The present invention thus provides products and pharmaceutical compositions which may be used for stimulating immune responses, and in particular T cell specific immune responses, in humans and/or other (non-human) subjects, which may be beneficial for (but are not limited to) preventing and/or treating diseases. As used herein, to treat a subject means to provide some therapeutic or prophylactic benefit to the subject. This may occur by reducing partially or completely symptoms associated with a particular condition. Treating a subject is not however limited to curing the subject of the particular condition.

In one aspect of the invention, there is provided a method of inducing an antigen-specific immune response in a subject, the method comprising delivering an effective amount of a peptide and/or a polyepitope string and/or a nucleic acid and/or an expression vector and/or a particle and/or a cell and/or a T cell and/or an agent and/or a pharmaceutical composition of the present invention, respectively, to a subject.

In one aspect of the invention, there is provided a peptide, a polyepitope string, a nucleic acid, an expression vector, a particle, a cell, a pharmaceutical composition, a T cell, an agent or a vaccine according, to the present invention, respectively, for use as a medicament, in particular for use in the treatment of cancer. The treatment may be combined with one or more additional treatments, in particular anti-cancer treatments, such as chemotherapy, radiotherapy or further immunotherapy.

In an aspect of the invention there is provided the use of a peptide, a polyepitope string, a nucleic acid, an expression vector, a particle, a cell, a pharmaceutical composition, a T cell, and agent or a vaccine of the present invention, respectively, for the manufacture of a medicament for the treatment of cancer.

In a further aspect, the invention provides a method of treatment of cancer in a subject, the method comprising administering to a subject in need thereof a therapeutically effective amount of a peptide and/or a polyepitope string and/or a nucleic acid and/or an expression vector and/or a particle and/or a cell and/or an agent and or a pharmaceutical composition and/or a T cell and/or a vaccine of any the present invention, respectively. The subject may be for example, but not limited to, a mammal or a primate. Preferably it is a human.

Diseases which may be treated in accordance with the invention comprise cancer.

Diseases which may be treated in accordance with the invention comprise haematologically derived malignancies such as multiple myeloma, mantel cell lymphoma, Hodgkin's lymphoma, T cell lymphomas, follicular lymphoma, Burkitt's lymphoma, T cell rich B cell lymphoma, diffuse large B-cell lymphoma (DLBCL), acute myeloid leukaemia, chronic myeloid leukaemia, myelodysplastic syndrome (MDS), in particular acute myeloid leukaemia (AML).

Diseases which may be treated in accordance with the invention comprise non-haematologically derived malignancies such as melanoma, lung, breast, gastric, kidney, prostate, ovarian, uterine, colorectal, liver, head and neck cancers and adenocarcinoma of the colon.

In a further aspect of the invention there is provided a method of detecting a cancer, the method comprising testing a sample obtained from a subject for the presence of
(a) a T cell or T cell line specific for a peptide of the invention, or
(b) an epitope or peptide of the invention, or
(c) an APC or tumour cell presenting an epitope or peptide of the invention on an MHCI molecule, or
(d) a TCR recognising the epitope or peptide of the invention, or
(e) activation of T cells (i.e. detection of IFNγ production and/or quantification of T cell numbers using pentamers/tetramers) against the epitope or peptide of the invention, or
(f) detection of peptide-specific T cells using an pMHC array.

Point (f) may also include capture antibodies and post-detection isolation and examination for function by IFNg ELISpot assays and CTL chromium-release assays.”

Peptide-MHC microarrays are known in the art (for example described in^{42,43}).

The method may comprise a preceding step of obtaining a sample from the subject.

The presence of any of features (a) to (e) may indicate a cancer. The T cell of (a) may be a CTL or a T_{H} cell. The presence of (a) may indicate the presence of an available repertoire. The healthy donor has T cells which can react to the epitope when it is presented to them. This suggests that a healthy donor who has not yet developed a cancer has the T cells available to react to the epitope of interest, in the present case in PASD1, and that these T cells have not been clonally deleted. The methods described herein may the used to diagnose a cancer in a subject. Further, detecting mRNA or protein expression from PASD1 can be used to detect tumour presence. Monitoring of PASD1 expression can provide minimal residual disease information about when a patient is going into or going to come out of remission. T cell numbers measured by tetramers by FACS or on the pMHC array as described herein indicate which patients, even at diagnosis when disease loads are high, have T cells which can recognise the epitope of interest and therefore will begood responders to conventional treatment (higher LAA expression has been associated with better responses in AML Ref: Guinn, B. A., Greiner, J., Schmitt, M. & Mills, K. I. (2009) Elevated expression of the leukaemia associated antigen SSX2IP predicts good survival in acute myeloid leukaemia patients who lack detectable cytogenetic rearrangements. Blood, 113, 1203-1204). It will further indicate those patients who will be susceptible for immunotherapy targeting specific epitopes. It will further help clinicians to decide which targets should be aimed at first whether multiple targets should be treated and when to change to another/other target(s) with disease progression or immune response(s). In addition the presence of specific T cell population(s) will provide prognostic information.

The inventors used peptide-MHC microarrays, as described in 42 and 43, to test whether AML patients had T cells which could recognize the P14 peptide on HLA-A2.

The products and methods described herein thus find utility in prognosis. Patients may be screened prior to treatment to identify those patients that will benefit or are most likely to benefit from immunotherapy stimulating T cells specific for a given PASD1 epitope described herein.

The waning of T cell numbers indicate which other epitopes could be targeted. It is believed that patients with multiple T cell responses are more likely to respond well to chemotherapy (which instigates cell death, release of antigens to the immune system, and inflammation, necessary for effective T cell responses).

The present invention thus provides methods for predicting a subject's susceptibility for an immunotherapy based on epitopes/peptides of the invention. For example, using the methods described herein a subject can be identified as being likely to respond to PASD1 based therapy, in particular a therapy based on one or more of the peptides and epitopes described herein.

The detection method may be used to monitor the progression of a cancer by performing the method on samples obtained from a subject at several time points, i.e. several days, weeks, months, or years apart. It may also be used to monitor a cancer in a subject in response to treatment. To monitor a cancer detection method described herein may be performed before and after treatment, or at several time points during the treatment.

In a further aspect there is provided a method of predicting the susceptibility of a subject for a treatment as described herein, the method comprising testing a sample obtained from a subject for the presence of
(a) a T cell or T cell line specific for a peptide of the invention, or
(b) an epitope or peptide of the invention, or
(c) an APC or tumour cell presenting an epitope or peptide of the invention on an MHCI molecule, or
(d) a TCR recognising the epitope or peptide of the invention, or
(e) activation of T cells (i.e. detection of IFN-γ production and/or quantification of T cell numbers using pentamers/tetramers) against the epitope or peptide of the invention, or
(f) detection of peptide-specific T cells using an pMHC array,
   wherein detection of any one of features (a) to (e) indicates the subject's susceptibility for said treatment. The subject may be a patient suffering from cancer.

The following techniques may be employed for testing for features (a) to (f) of any of the methods above.

### Methods:

1) IFN gamma or granzyme ELISPOT assays
2) Intracellular cytokine staining
3) Streptamer/Tetramer/pentamer staining
4) Loss of CCR7 homing marker to indicate migration of T cells out of lymph nodes to blood and target sites.
5) CTL assays in which target cells are chromium labelled and chromium release indicates effective killing by the T cells.
6) Loss of CCR7 homing marker to indicate migration of T cells out of lymph nodes to blood and target sites.
7) Th17 and Treg numbers, production of cytokines by APCs and T cells, peptide presentation, markers of T cell activation, measurements of effector T cells numbers, phenotypic markers of anergy.

In a further aspect there is provided a method of monitoring an anti-PASD1 immune response in a subject which comprises detecting in a sample obtained from the subject the presence of:
1) an epitope or peptide or polyepitope string as described herein, or
2) a T cell or a T cell line as described herein
3) a T cell receptor as described herein,
   wherein the presence of said epitope, peptide, polyepitope string, T cell, T cell line or T cell receptor indicates a anti-PASD1 immune response.

In a further aspect of the invention there is provided a method of staging a cancer, the method comprising testing a sample obtained from a subject for the presence of
(a) a T cell or T cell line specific for a peptide of the invention, or
(b) an epitope or peptide of the invention, or
(c) an APC or tumour cell presenting an epitope or peptide of the invention on an MHCI molecule, or
(d) a TCR recognising the epitope or peptide of the invention, or
(e) activation of T cells (i.e. detection of IFNγ production and/or quantification of T cell numbers using pentamers/tetramers) against the epitope or peptide of the invention, or
(f) detection of peptide-specific T cells using an pMHC array.

The methods and products described herein may thus be used to predict the susceptibility of a subject to treatment and as well as the response of the subject to the treatment.

### Examples

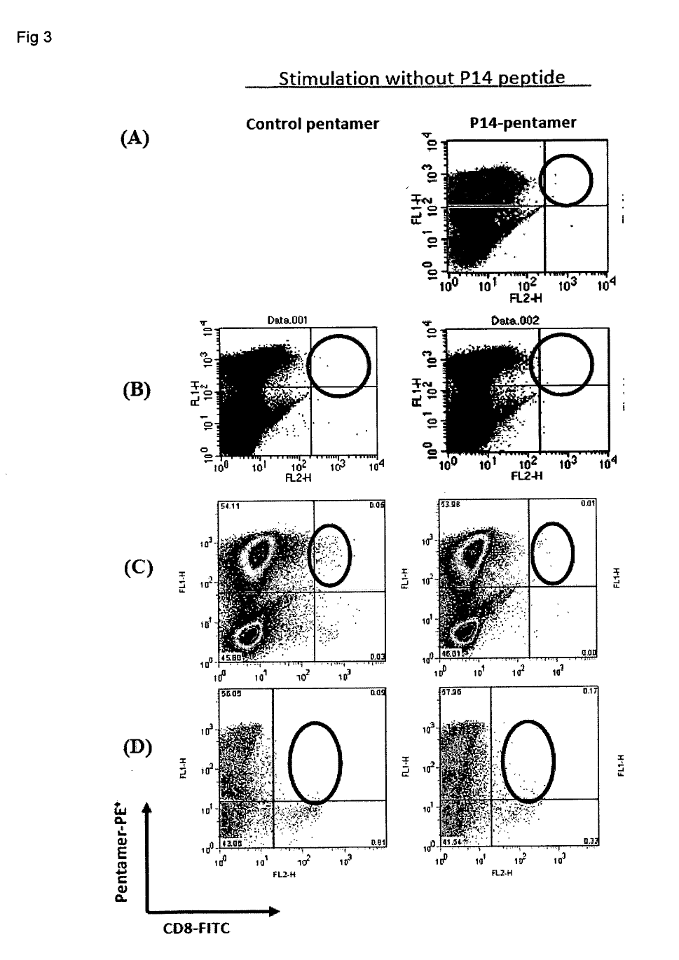

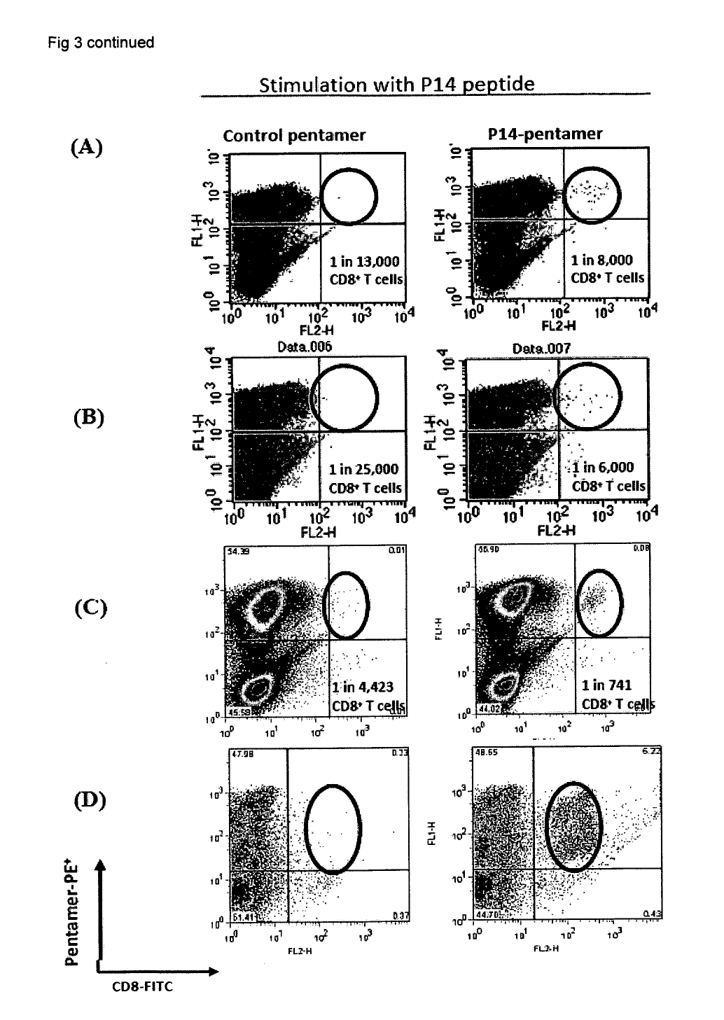

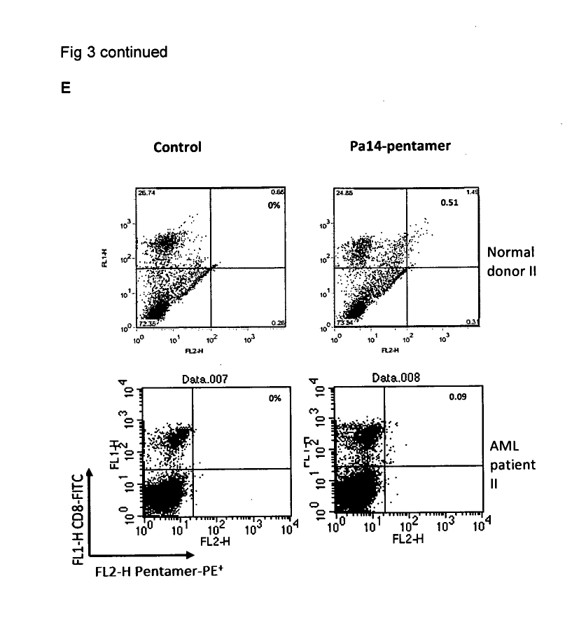

PASD1 is a good target for immunotherapy due to its restricted expression. It is a cancer-testis antigen which is expressed only in immunologically protected sites such as the placenta and testes and with little or no expression in normal tissues. PASD1 is expressed in one-third of AML patients at presentation, and as such is the most frequently expressed CT antigen in AML described to date. PASD1 is also recognised by sera from CML patients and is expressed in 1 of 6 patients at presentation and was expressed in JURKATS, a T cell leukaemia cell line. PASD1 also shows expression in some solid tumours as suggested by its expression in solid tumour cell lines, such as Hn5 (a head and neck line), H1299 (a lung cancer cell line) and SW480 (colon cancer). In addition the data (FIG. 3c & D) shows that PASD1-specific T cells can be expanded from a colon cancer patient. PASD1 has already been shown to be expressed in a number of haematological malignancies including diffuse large-B cell lymphoma²⁶ and multiple myeloma²⁷.

The inventors have now used web-based algorithms (SYFPEITHI and BIMAS) and reverse immunology to identify HLA-A*0201 binding epitopes within PASD1.

The PASD1 sequence which the inventors isolated from the testis library was given the NCBI data base id of AY623425 (SEQ ID NO 38, with predicted amino acid sequence SEQ ID NO 39: Accession number AAT49049.1). This sequence was used for the prediction of P4-P16 with limitation to epitopes which showed 40% or less similarity to known proteins in any other eukaryotes.

The inventors found that some wild type peptides failed to bind to HLA-A2 molecules on T2 cell lines with any notable frequency. The inventors modified a single anchor residue within the nonomers (Table 2) and re-assessed immunogenicity. The inventors made sure that every modified peptide did not match, with more than 40% amino acid sequence similarity, any known eukaryotic proteins. The inventors then assessed whether these peptides had improved binding to HLA-A2 molecules on T2 cells and whether they could induce IFNγ secretion from autologous T cells stimulated with peptide loaded monocyte-derived dendritic cells.

### Materials and Methods

### Identification of Heteroclitic Peptides Using Algorithms

The inventors used SYFPEITHI³³ and BioInformatics & Molecular Analysis Section (BIMAS)³⁴ algorithms to identify ten nonamers predicted to bind HLA-A2 (Table 2). These were specific to PASD1 and no other known eukaryotic proteins. Peptides 1-3 (P1: KIQEQLQMV, P2: FLTKGQQWI, P3: VLQKSIDFL) were located in the human CLOCK gene and peptides 4-10 (P4-P10) in the carboxy region of PASD1_v1 and PASD1_v2. When peptides P1-10 were tested for their ability to stabilise HLA-A2 molecules on the surface of T2 cells³⁵ in FACS based assays, P1 alone showed binding. The poor binding in T2 assays was reflected by the low SYFPEITHI scores of the epitopes. The inventors made a single amino acid change (at either position 2 or 9) to P4-P10 to see whether the binding of the peptides to the MHC groove^{36,37} could be enhanced. Peptide analogues with increased SYFPEITHI scores and low homology to known eukaryotic proteins (except PASD1) were selected for study, these were denoted P11, P12, P13, P14, P15 and P16. These ‘peptide derivatives’ were custom synthesized and tested for binding ability in T2 assays.

### Peptides

The HLA-A*0201-restricted P8, P14, P15 and P16 peptides, WT1.37⁹ peptides and the HLA class II-restricted p30 (FrC-derived: TTFNNFTVSFWLRVPKVSASHLE)³⁸ peptides were synthesized commercially and supplied at >95% purity (PPR Ltd, Southampton, U.K.).

### Patients and Healthy Donors

Healthy donor lymphocytes were obtained from buffy coats (National Blood Transfusion Service, Tooting, London, UK). All patient samples were received following informed consent and following local ethics committee approval. Primary AML blasts were obtained from the peripheral blood of adult patients with high-count AML at diagnosis and prior to the initiation of chemotherapy. Peripheral blood mononuclear cells (PBMCs) were obtained from AML patients in complete morphological and cytogenetic remission. Cryopreserved bone marrow samples or peripheral blood stem cell harvests (PBSCH) from patients were supplied by the Stem Cell Laboratory, King's College Hospital, London, UK. PBMCs were purified by Histopaque density gradient centrifugation (Sigma) and cryopreserved in X-VIVO 15, 10% DMSO (Sigma) and 50% Human AB serum (Sigma). All primary cells were cultured in X-VIVO 15 medium while all AML cell cultures were additionally cultured with recombinant human SCF (20 ng/ml) and IL-3 (10 ng/ml) (R&D Systems, UK). CD3′ and CD8⁺ cells were obtained from healthy donor PBMCs using Negative Isolation Kits (Miltenyi Biotec) and CD4⁺ cells were depleted from effector cell populations by positive selection (Dynal, Oslo, Norway) as per manufacturer's instructions. CD14⁺ cells were purified from remission bone marrow using positive selection using MACS CD14 beads (Miltenyi Biotec). All separations using Macs beads were carried out with an Automacs machine (Miltenyi Biotech). Healthy donor samples found to be HLA-A2 positive by FACS analysis were sent for subtyping at the Anthony Nolan Laboratories, Royal Free Hospital, London. HLA-A*0201 samples were subsequently used in T cell stimulation assays. Where possible negative selection was performed to obtain effector cells, however when isolating CD3⁺ cells from non-remission AML samples, it was necessary to positively isolate CD3⁺ cells from thawed presentation samples or using CD3 Macs microbeads (Miltenyi Biotec) as per manufacturer's instructions.

Four AML patients, two with PASD1⁺ cells (Patient I and II) and two with PASD1⁻ tumour cells (Patient III and IV), one patient with colon cancer (Patient V), one patient with head and neck cancer (Patient VI) and one patient with prostate cancer (Patient VII) were analysed (Table 1). PASD1 expression was confirmed by PASD1-specific RT-PCR as described previously¹⁹. Informed consent in accordance with the Declaration of Helsinki was obtained from all healthy subjects and patients prior to sampling.

### Flow Cytometry and Pentamer Staining

For the analysis of cell surface molecules, cells were washed with cold wash buffer (HBSS, 1% FBS, 0.1% sodium azide) and resuspended in the residual volume. Cells were incubated for 30 minutes at room temperature with directly conjugated antibodies and matched isotype controls were included for each sample. Cells were then washed twice and resuspended in 300-500 μl of wash buffer.

For detection of IFNγ, Brefeldin A was added to T cells 12 hours prior to intracellular staining, to a final concentration of 1 mg/ml. Effector cells were washed with PBS and stained with CD8-PE or CD4-PE antibody for 30 minutes at room temperature. Stained cells were then washed twice with HBSS, and resuspended in the residual volume. 100 μl of fixation solution (Caltag Laboratories, UK) was added to each tube and samples incubated for 15 minutes at room temperature. Cells were then washed with cold HBSS, 1% FBS 0.1% sodium azide and resuspended in the residual volume. 100 μl of permeabilisation medium (Caltag Laboratories) and 5 μl of IFNγ-FITC was added to each tube, and incubated for 20 mins at room temperature. Finally, cells were washed and resuspended in 300-500 μl HBSS ready for FACS analysis. All antibodies and isotype controls were purchased from (Becton Dickinson, Oxford, UK) except HLA-A2 (from Serotec).

Assessment of peptide specific T cells was carried out by staining 10⁶ effector cells with 10 μl of PE labeled, HLA-A*0201 pentamers (custom made by Proimmune) for 10 minutes, at room temperature, in the dark. Cells were then washed and co-stained with CD8-FITC for 20 minutes at room temperature. The lymphocyte gate was selected according to FSSCH/SSCH and 50,000 events acquired. Staining with control pentamers was carried out for each sample.

### T2 Assays

We used the T2 cell line³⁹ to assess binding of the peptides to HLA-A2. The T2 cell line is TAP deficient and exhibits inefficient processing of endogenous antigens. Peptide binding stabilises HLA-A*0201 molecules, increasing their level on the cell surface in a dose dependent manner which can be detected by FACS analysis using an anti-HLA-A2 monoclonal antibody. T2 cells were seeded in round bottomed 96 well plates at a density of 3×10⁵ per well in 100 μl of medium (RPMI, 10% FCS, P/S). Peptides were added in 100 μl of serum free medium to give a final concentration of between 100-0.05 μM. Control wells with no peptide were also seeded. T2 cells were incubated overnight, washed and stained with 5 μl of anti-human HLA-A2-FITC antibody (Serotec). Stabilisation of HLA-A2 molecules on the surface of T2 cells were compared to unpulsed control T2 cells. To determine longevity of binding, peptide pulsed T2 cells were washed three times and replated in fresh medium. Aliquots of cells were removed at different time points after removal of the peptide, and by flow cytometry as described.

Epitope Specific T Cell Responses in HLA-A2 Normal Donors after Stimulation with Autologous Peptide Loaded DCs

PBMCs were prepared from healthy donor buffy coats as described above. Monocytes were obtained from newly sourced buffy coats, or cells thawed at 10⁶/ml in warm X-VIVO medium, 1% human AB serum and plated in 90 mm TC dishes. Plates were incubated at 37° C. for at least 4 hours and non adherent cells removed by gently washing with media or HBSS or CD14⁺ cells were positively selected as described. The remaining T cell enriched cells were cryopreserved for use as effectors in later assays. The CD14⁺ fraction/adherent cells were cultured in IL-4 (1000 IU/ml) and GM-CSF (800 U/ml) for 5 days to induce differentiation to a dendritic cell (DC) phenotype. On day 3 fresh IL-4 and GM-CSF were added. On day 5 TNFα (10 ng/ml), IL-6 (1000 U/ml) and IL-1β (10 ng/ml) all from R&D systems UK and PGE₂ (1 μg/ml) (from Sigma, UK) were added to plates. On day 6 DCs were harvested from plates and washed with HBSS to remove residual cytokines before use in immunological experiments. Analysis of DC phenotype was carried out by flow cytometry.

### ELISA Assays

IL-2 and IFNγ levels were determined with Duo set ELISA Development System (R&D Systems), according to the manufacturer's instructions. Supernatants were collected at various time points (days 3, 7, 10 and 14) in order to detect peak cytokine levels.

Stimulation of T Cells with PASD1 Peptide Analogues

After 24 hours in maturation cytokines, DCs were incubated with peptide (50 μg/ml) for 4 hours. PBMCs or CD3⁺ cells from healthy donors or patients with solid tumours were seeded into a 12-well plate, in X-VIVO 15 at a density of 2×10⁶/ml. Peptide pulsed, monocyte derived DCs were washed and prepared at 2×10⁵/ml. PBMC cultures received peptide pulsed DCs at a stimulator:effector ratio of 10:1, or in the case of unstimulated controls, medium only. IL-7 was added to cultures at a final concentration of 10 U/ml on day 3. Cultures were restimulated by addition of peptide pulsed DCs on, day 7, and in some cases day 14, together with IL-7 and IL-2 (both at 10 U/ml). 200 μl of culture supernatants were collected at various intervals throughout the culture period and replaced with fresh medium. In the case of AML patient cultures, T2 cells were pulsed with peptide and used as stimulators in the same way as autologous DCs described above. Culture supernatants were analysed for IFNγ content by ELISA. After the 2-3 week culture period, stimulated effector cells were washed and analysed by pentamer staining, intracellular cytokine staining or ELISPOT assays.

Epitope Specific T Cell Responses in Cancer Patients after Stimulation with Peptide Loaded T2 Cells.

Due to the absence of healthy monocytes from presentation haematological samples and the absence of peripheral blood samples from solid tumour patients, we used T2 cells loaded with peptide to stimulate T cell responses against P14, P15 and P16. The T2 line was first examined for immune stimulatory molecules by FACS analysis. T2 cells expressed MHC class I, CD40, CD80, CD54 and CD86 but were found to be MHC class II negative (data not shown). T2 cells were cultured in serum free conditions to reduce the non-human antigens present in the FCS. T2 cells were incubated with peptide for 4 hours, washed and irradiated and seeded in 96 well plates. Purified T cells (Miltenyi) from patients were added and IFNγ secretion or the expansion of CD8⁺ epitope specific T cells measured using pentamers.

### Construction of DNA Vaccines

Construction of the p.DOM plasmid containing the gene encoding the first domain of FrC of tetanus toxin (DOM, TT865-1120) with a leader sequence derived from the VH of the IgM of the BCL1 tumour at the N-terminus has been previously described³¹ Additional DNA vaccines were constructed encoding either PASD1 691-699 (pDOM.P8 or pDOM.P14), PASD1 587-595 (pDOM.P15) or PASD1 587-595 (pDOM.P16) peptides fused directly 3′ to DOM. p.DOM.epitope vaccines were constructed by PCR amplification using p.DOM as template with the forward primer 5′-TTTTAAGCTTGCCGCCACCATGGGTTGGAGC-3′ and the following reverse primers:

P8 reverse primer:
5′-ATATGCGGCCGCTTAGATATCAGACAACTCT
TGCCAAAGCCGGTTACCCCAGAAGTCACG-3′;
P14 reverse primer:
5′-ATATGCGGCCGCTTATGAATCAGACAACTCT
TGCCAAAGCCGGTTACCCCAGAAGTCACG-3′;
P15 reverse primer:
5′-ATATGCGGCCGCTTACACAGATACGTCACGT
GGGTTTATCAGGTTACCCCAGAAGTCACG-3′;
P16 reverse primer:
5′-ATATGCGGCCGCTTACACAGATACGTCACGT
GGGTTTACCAGGTTACCCCAGAAGTCACG-3′.

The PCR product was gel purified, digested using HindIII and NotI restriction sites and cloned into the expression vector pcDNA3 (Invitrogen, Paisley, U.K). Restriction sites within primers are shown in bold and PASD1-peptide encoding sequences are italicised while modified sequences are underlined. Integrity of the inserted sequence was confirmed by DNA sequencing and translated product size was checked in vitro using the TNT T7 coupled reticulocyte lysate system (Promega, Southampton, U.K.).

### HHD Transgenic Mice

HHD mice express a transgenic chimeric monochain MHC class I molecule in which the COOH-terminus of human β2-microglobulin is covalently linked to the NH₂-terminus of chimeric HLA-A2 α1 and α2 domains fused with the murine H-2D^{b} α3 domain. These mice lack cell-surface expression of mouse endogenous H-2b class I molecules due to targeted disruption of the H-2D^{b} and mouse β2-microglobulin genes³⁹.

### Vaccination Protocol

HHD mice at 6 to 10 weeks of age were injected intramuscularly (i.m.) into both quadriceps with a total of 50 μg DNA in saline solution on day 0. Unless stated otherwise mice were boosted with the same DNA vaccine delivered with in vivo electroporation on day 28 as previously described⁵. Animal experimentation was conducted within local Ethical Committee and UK Coordinating Committee for Cancer Research (London, U.K) guidelines under Home Office License.

### Mouse IFNγ-ELISpot

Vaccine-specific IFNγ secretion by lymphocytes from individual mice was assessed ex vivo (BD ELISpot Set, BD PharMingen, San Diego, Calif.) on day 14 or 36, as described previously with some modifications¹¹. Briefly, viable lymphocytes were selected from splenocyte preparations by density centrifugation. Cells (2-4×10⁵ cells/well) were incubated in complete medium (RPMI 1640, 1 mM sodium pyruvate, 2 mM L-glutamine, non-essential amino acids (1% of 100× stock), 50 μM 2-mercaptoethanol, 100 U/mL penicillin, 100 μg/mL streptomycin, (all Invitrogen) with 10% heat-inactivated foetal calf-serum) with either WT1.37 (irrelevant), P8, P14, P15 or P16 peptides to assess CD8⁺ T-cell responses, or with p30 peptide to assess CD4⁺ T cells. Samples were plated in triplicate; control samples were incubated without peptide or with an irrelevant HLA-A2-binding peptide (WT-1 126-134). Data are expressed as the frequency of spot-forming cells (SFCs) per million lymphocytes. For analysis of peptide-specific T-cell sensitivity, splenic lymphocytes from immunized mice were incubated with a range of PASD1 peptide concentrations and the frequency of specific cells assessed by ELISpot analysis as described. The number of SFC/million cells at the peptide concentration inducing the greatest response was assigned a value of 100%. For each peptide concentration tested the % maximal response was then calculated by the formula: (experimental SFCs per million cells/maximal SFCs per million cells)×100% for each individual animal.

### Cell Lines

Cells used as targets in murine cytotoxic T lymphocyte (CTL) assays were the human leukemia lines K562 (PASD1⁺HLA-A*0201⁺), H1299 (PASD1⁺HLA-A*0201⁻) or SW480 (PASD1⁺HLA-A*0201⁺) either alone, or retrovirally-transduced with HHD DNA using standard methods. The mouse cell line RMA-HHD was used as a murine PASD1_v2 negative cell line control.

### Murine Cytotoxic T Cell Expansion and Detection

For the generation and maintenance of CTL lines, mice were sacrificed at the indicated time points and cell suspensions made from each spleen. Splenocytes were washed and resuspended in 10-15 mL complete media per spleen in upright 25-cm² flasks together with P8, P14, P15 or P16 (100 nM or 1 μM) peptides. Following 7 days of stimulation in vitro, cytolytic activity of the T-cell cultures was assessed. For further cycles of in vitro re-stimulation, CTL were washed, resuspended at 3×10⁵/mL with 2.5×10⁶/mL syngeneic splenocytes pre-incubated for 1 hour with the relevant peptide at 1 μM, washed 4 times in unsupplemented RPMI 1640 (Invitrogen) and irradiated at 2,500 rad. Recombinant human interleukin-2 was added to cultures at 20 IU/mL (IL-2; Perkin-Elmer, Foster City, Calif.) and cells were incubated at 2 mL/well of a 24-well plate. Subsequent cycles of in vitro re-stimulation were carried out similarly every 7-10 days. Specific cytotoxic activity was assessed by standard 5 hour ⁵¹Cr release assay as previously described⁹.

### Results

### Variants of PASD1 Epitopes P1-P10 Stimulate T Cell Responses

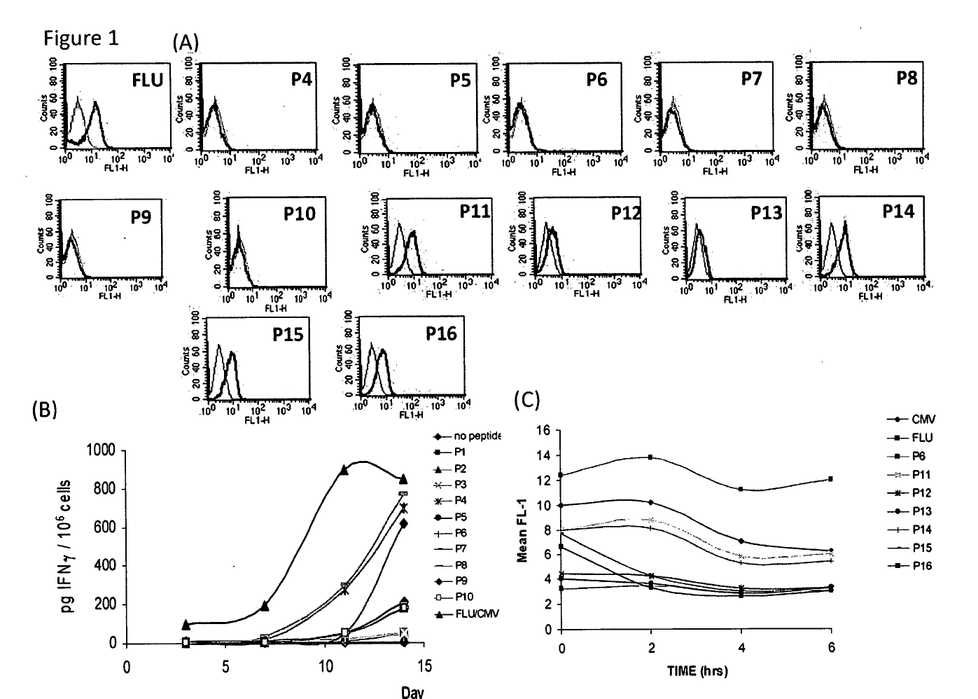

The inventors focused these studies predominantly on the carboxy region of PASD1, which was recognised by AML patient sera. They used two algorithms to identify HLA-A2 binding sequences and only studied those peptides which were specific to PASD1 and no other known eukaryotic proteins (as determined by BLAST searches). The inventors examined the capacity of P1-P10 to stabilise HLA-A2 on TAP-deficient T2 cells. None of the wild type epitopes (n=10) except P1 examined bound to HLA-A2 above background levels (FIG. 1A). Although in one of three normal donors tested P4, P8 and P9 peptides generated some IFNγ responses following stimulation with autologous DCs loaded with wild type peptide (FIG. 1B). However the SYFPEITHI scores were low and modification of the anchor residues on each of the wild type peptides which had induced IFNg from normal donor T cells (P4, P8 and P9) were examined to determine whether the inventors could improve the MHC class I binding and thus extend the time for which the peptide is seen by the TCR. All of the peptide analogues showed detectable binding to HLA-A2 in T2 assays, with P11 and P14 showing the greatest stabilisation of HLA-A2 molecules as determined by mean fluorescence (FIG. 1C). The duration of binding of the modified peptides to HLA-A*0201 on T2 cells were extended and ranged from 2-8 hours. The wild type P6 peptide was used as a negative control in this assay as it had the poorest binding in all T2 binding assays.

### PASD1 Modified Peptides can Stimulate Normal Donor T Cells

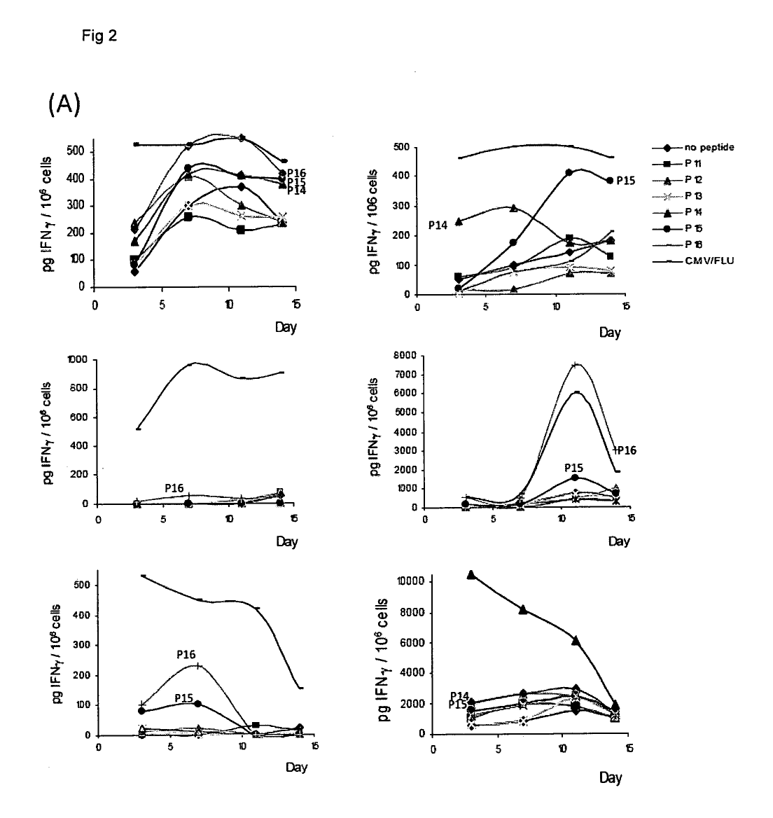

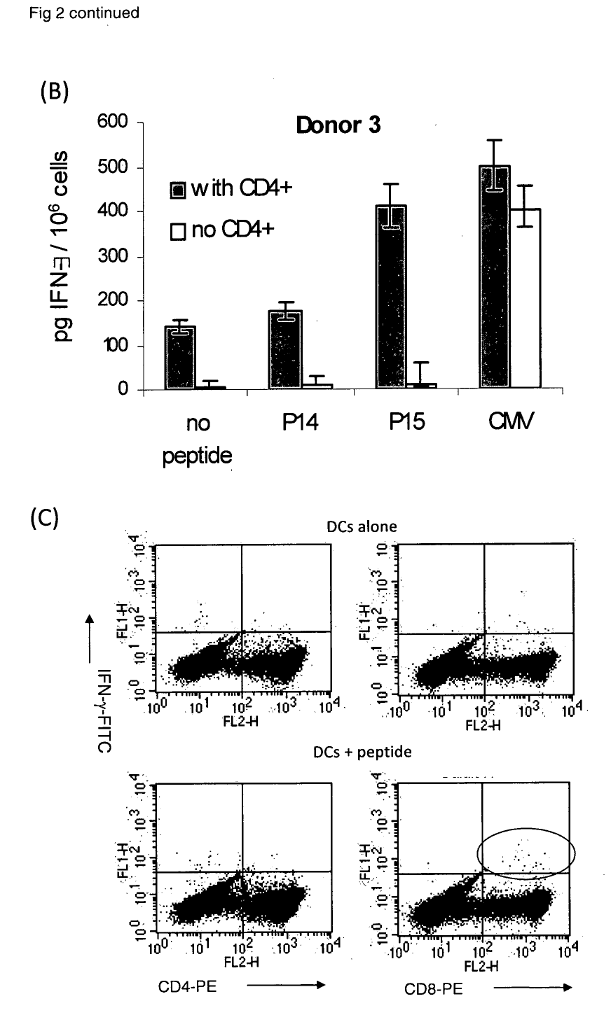

The inventors examined the capacity of the modified peptides P11-P16 to induce IFNγ responses from T cells from six normal donors. Peptides P14, P15 and P16 led to the highest levels of IFNγ production from most donors (FIG. 2A). It was hoped that the peptide analogues were stimulating a CD8⁺ response, since they were based on a class I binding motifs. To confirm this, some of the experiments were repeated using CD4⁺ depleted effector cells stimulated with P14, P15, P16. Secretion of IFNγ was almost completely abolished in these cultures, with only FLU or CMV stimulating significant cytokine levels (FIG. 2B). This shows that CD4+ T cells played a role in the response although only a class I peptide was provided to the dendritic cells. Therefore it was unclear which cells were producing the IFNγ. Intracellular IFNγ staining was carried out on CD3⁺ stimulated cultures with either CD8⁺ or CD4⁺ co-staining to determine which subset were responding. After multiple ex vivo stimulations intracellular IFNγ production was detected in CD8+ cells, illustrating that the IFNγ producing population were CD8⁺ cells (FIG. 2C). Therefore, despite the response being dependent on the presence of CD4⁺ cells in the culture, the responding cells within the cultures were CD8⁺ T cells.

Pentamers for P14 and P15 were generated by custom synthesis. Three donors which had shown an IFNγ response to P14 and P15 were selected for these assays. 10⁷ purified CD3⁺ cells were stimulated with peptide pulsed DCs every seven days. Prior to each stimulation, cells were resuspended and samples taken for pentamer staining. In two of the four normal donors tested a population of P14 and P15 no pentamer positive cells were detectable after four stimulations. In two donors, a small population of pentamer positive T cells were detectable after four stimulations with P14 or P15 (FIG. 3A+E).

PASD1-Specific CD8⁺ T Cells were Stimulated in AML Patient Samples

Purified CD3⁺ T cells from four HLA-A*0201 AML patients, three of which had PASD1 positive AML blasts were used in peptide stimulations. The T cells were stimulated with T2 cells loaded with P14 and P15, as no autologous APCs were available. Prior to each restimulation, cells were resuspended and aliquots taken for pentamer staining. In Patient 1, P15 pentamer positive T cells were visible in the absence of stimulation, suggesting that the corresponding wt peptide had already primed a T cell response in vivo (data not shown). After two stimulations with P14 or P15 loaded T2 cells, the frequency of pentamer positive CD8⁺ cells, although small, was increased (P15 data not shown). In two of the four patients analysed P14 pentamer positive cells were not detectable above background levels in the absence of peptide stimulation. P14 stimulation increased the level of P14-pentamer positive cells to 0.02% of the CD8⁺ cells after two rounds of ex vivo stimulation with P14 (FIG. 3B+E).

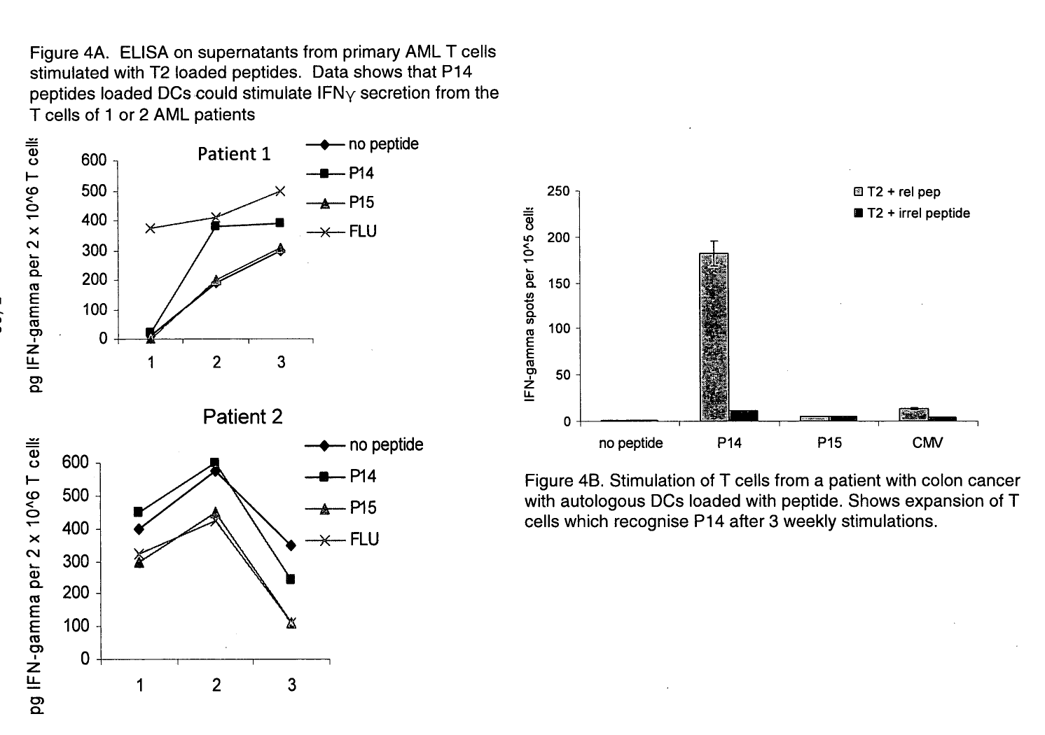

The percentage of P15 pentamer positive cells in the absence of peptide stimulation was detectable above background at a frequency 0.01% of the CD8⁺ T cells. This increased to 0.02% of the CD8⁺ T cells after two stimulations. A third stimulation did not increase the percentage of pentamer positive cells with either peptide, due to activation induced T cell death which has also been previously reported by others when stimulating AML T cells with other TAAs^{46,47}. In cultures from AML Patient I and II, P14 pentamer positive cells were undetectable after stimulation with DCs alone, but increased to 0.5% (Patient I) and 0.09% (Patient II) of the CD8⁺ cells after P14 peptide stimulation. Stimulations did not increase the numbers of pentamer positive cells in either of these patient cultures. IFNγ was secreted by T cells from both of these expanded P14-specific populations as determined by ELISA (FIG. 4A).

In cultures set up with CD3⁺ cells from AML Patient 3 (collected during remission) and Patient 4 (non remission), no pentamer positive cells were detected, even after three stimulations (data not shown). In Patient 4 cultures, only P14 stimulation was carried out, due to a limited number of available CD3⁺ cells. After four stimulations, few viable cells remained in both patient cultures, therefore no further analysis was carried out.

PASD1-Specific T Cells can be Expanded from Solid Tumour Patients

There have been no reports of PASD1 expression in primary head and neck, prostate or colon cancer primary cells to date, but the head and neck cancer cell line Hn5¹⁹, the lung cancer line H1299¹⁹ and the colon cancer cell line SW480²⁴ are PASD1 positive. This raises the possibility that some solid cancer patients may be able to raise a response to the PASD1 peptides. Cells used in these experiments were isolated from leukophoresis samples taken several months after the cessation of treatment. Negatively purified CD3⁺ T cells were stimulated with autologous monocyte derived DCs as described previously. Pentamer analysis was carried out after each stimulation. No pentamer positive cells were detected at anytime in the T cell cultures from the prostate cancer patient, even after four stimulations. T cell cultures from the head and neck patient had a low frequency of P14 pentamer positive cells at 0.02% of CD8⁺ cells, but these were not expanded by P14 peptide stimulation. P15 pentamer positive cells were also detected at the same low frequency in the absence of peptide stimulation, but three rounds of P15 peptide stimulation expanded these marginally to 0.06% of the CD8⁺ population (data not shown). A further fourth stimulation did not expand this further.

In contrast, a large expansion of P14 pentamer positive cells was seen in cultures from the colon cancer patient (FIGS. 3C and D). Background staining of cells was visible with the control pentamer, so all values were corrected for this. P14 pentamer positive cells were detectable after stimulation with DCs alone at a relatively high frequency of 0.09% CD8⁺ cells. Three rounds of P14 peptide stimulation expanded the number of pentamer positive cells to a frequency of 0.11% of CD8⁺ T cells (FIG. 3C). A fourth stimulation further increased the percentage of pentamer positive cells to a high frequency of 13.6% of the CD8⁺ T cell population (FIG. 3D). Specific IFNγ secretion was detectable by ELISpot in this patient (FIG. 4B) from T cell stimulated with P14 peptide, but not irrelevant or P15 peptide'. It was notable that normal donors often took more rounds of stimulation to expand PASD1-specific T cells to a detectable level.

In Vivo Assays Using HHD Mice Demonstrate that P14 is Recognised and Confers Immune Responses Against the Wild Type P8 Peptide

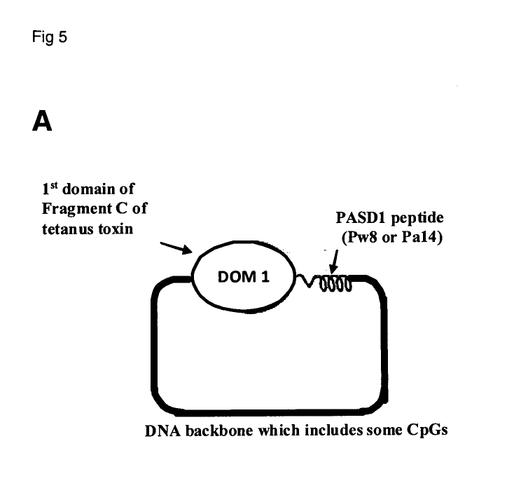

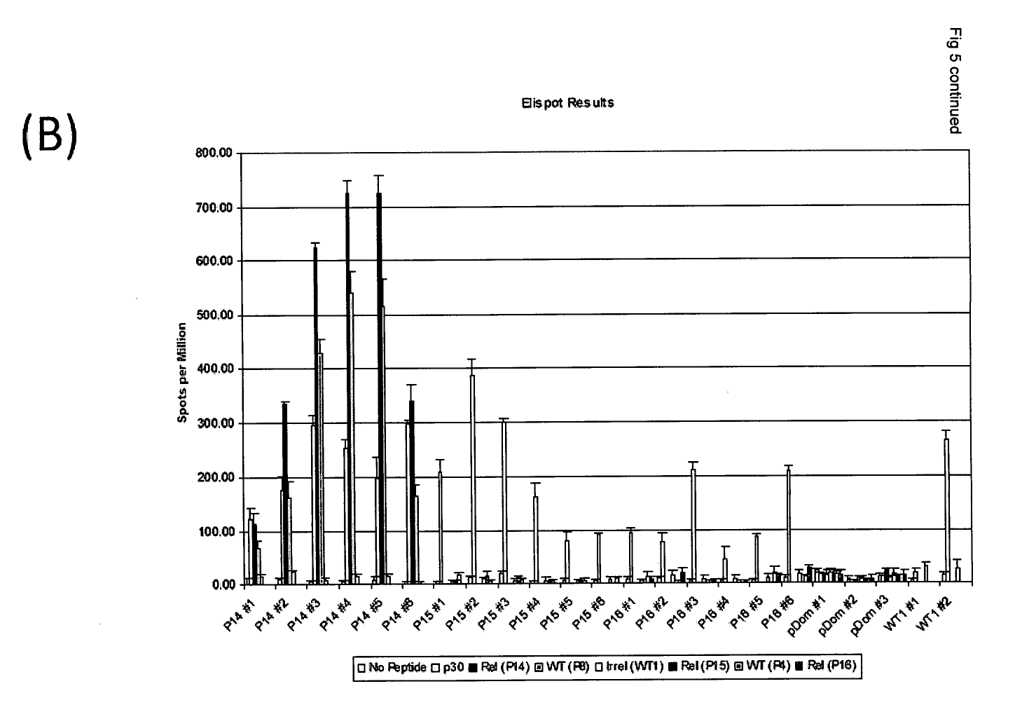

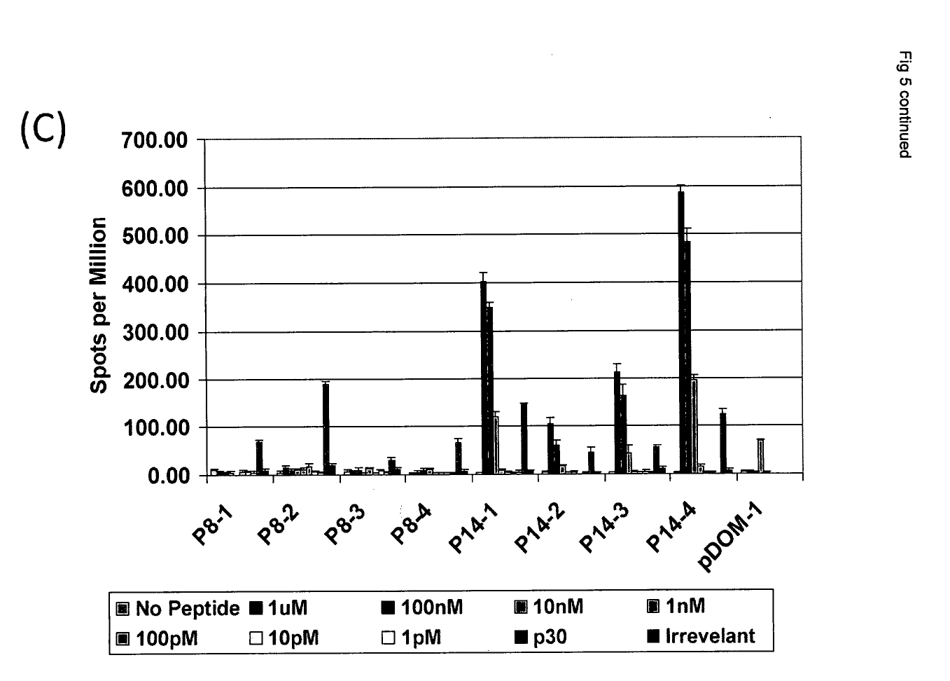

HHD mice were injected with pDOM.P14, P15 or P16 vaccines (FIG. 5A) and 14 days later examined by ELISpot to assess their responses to the modified peptide they were immunised against and its wild type counterpart. PASD1_v2 is not expressed in mice²⁴, unlike the common region of PASD1, and so prime only experiments were enough to generate T cell responses against P14. We found that only mice injected with pDOM.P14 could induce IFNγ secretion (FIG. 5B) while pDOM.P15 and pDOM.P16 vaccines could not. In addition we found that substantial but lower responses against wild type P8 peptide were induced and this result was highly reproducible. P30 responses indicated the operational integration of all the pDOM-epitope vaccines used. We examined how effective pDOM.P8 was at priming T cell responses in vivo in comparison with pDOM.P14 and found pDOM.P8 to be poor (FIG. 5C). CTL assays of T cells generated in prime experiments showed that a similar capacity to kill P14 and P8 loaded targets could be achieved once mice had been primed with pDOM.P14 (FIG. 5D).

CTL Lines were Capable of Killing Exogenous and Endogenous Modified and Wild Type Peptide

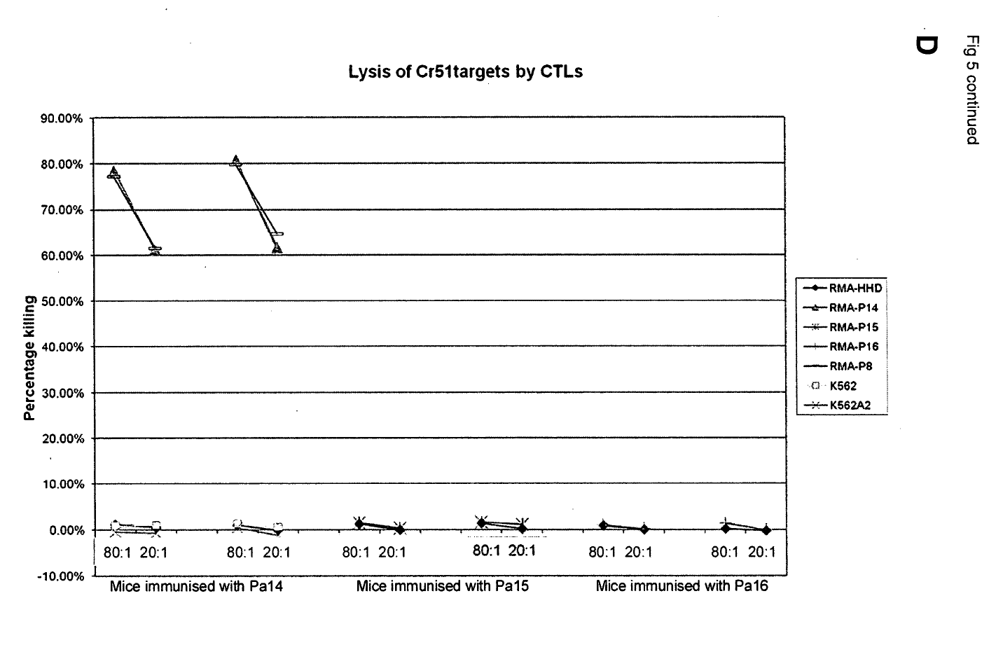

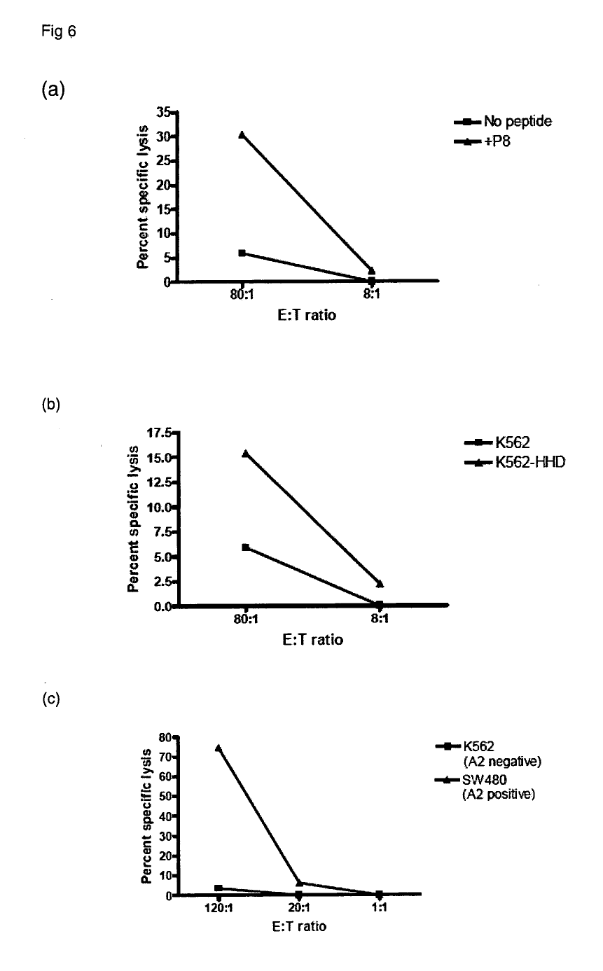

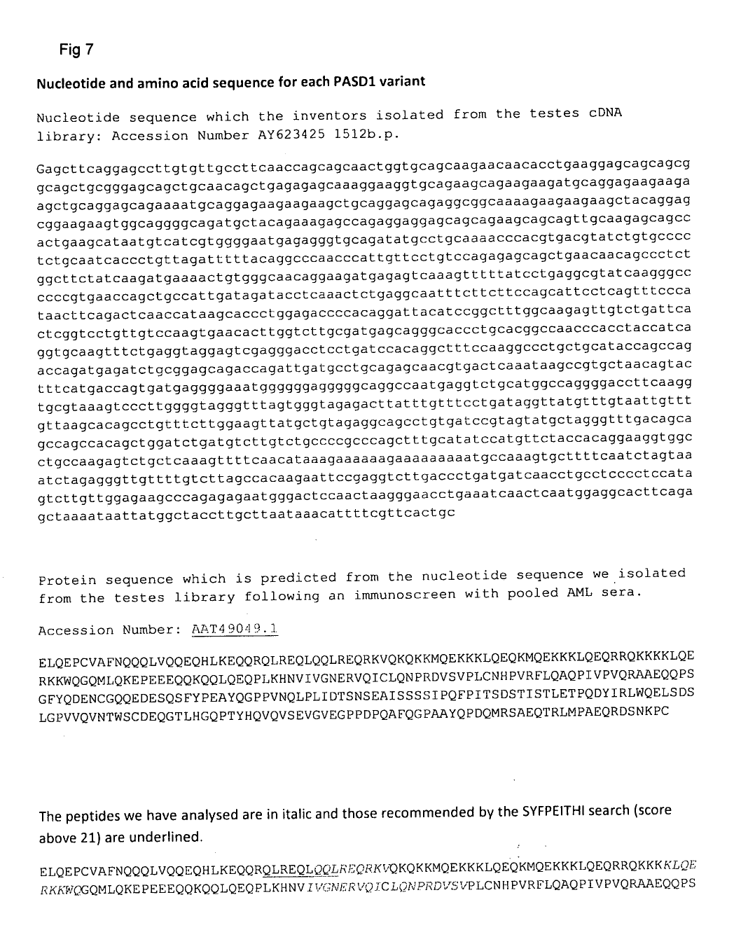

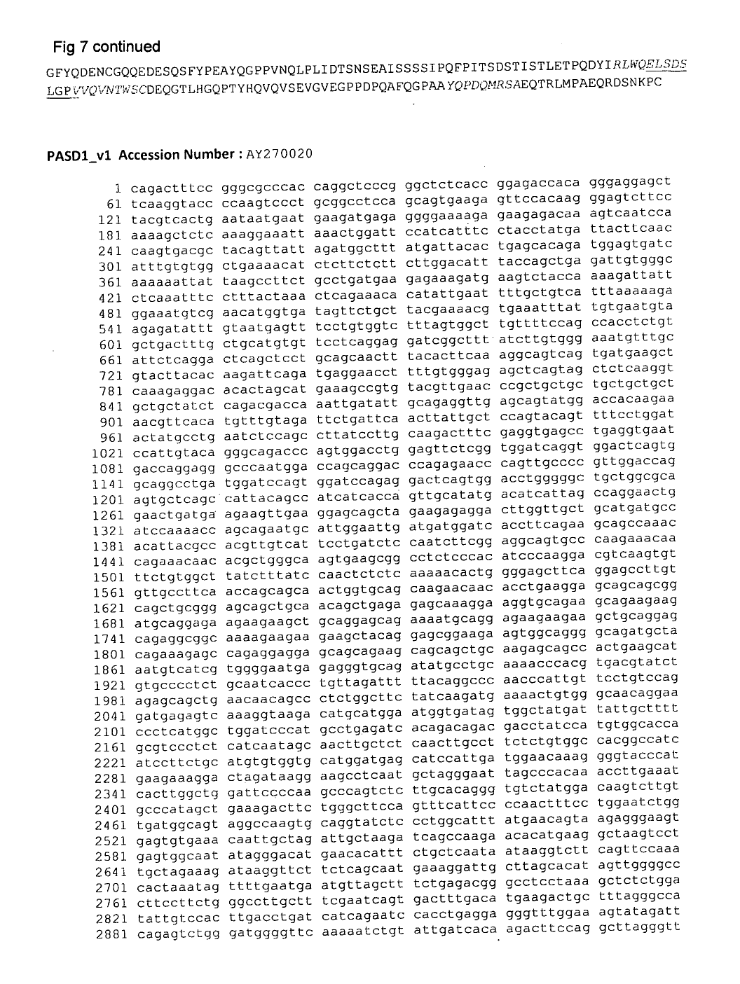

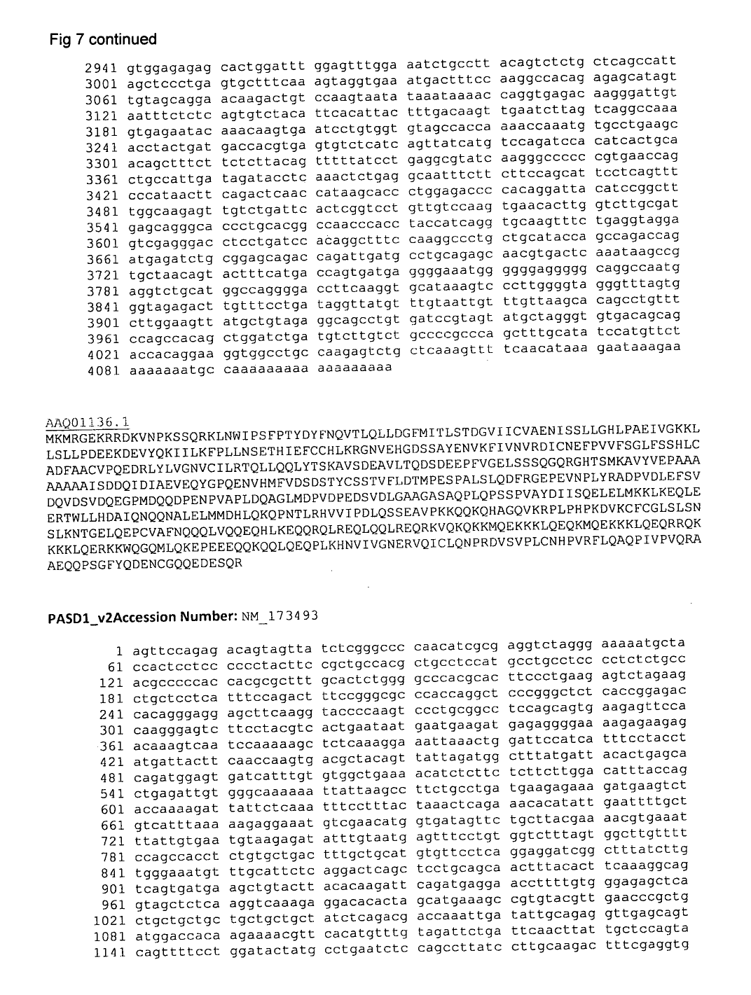

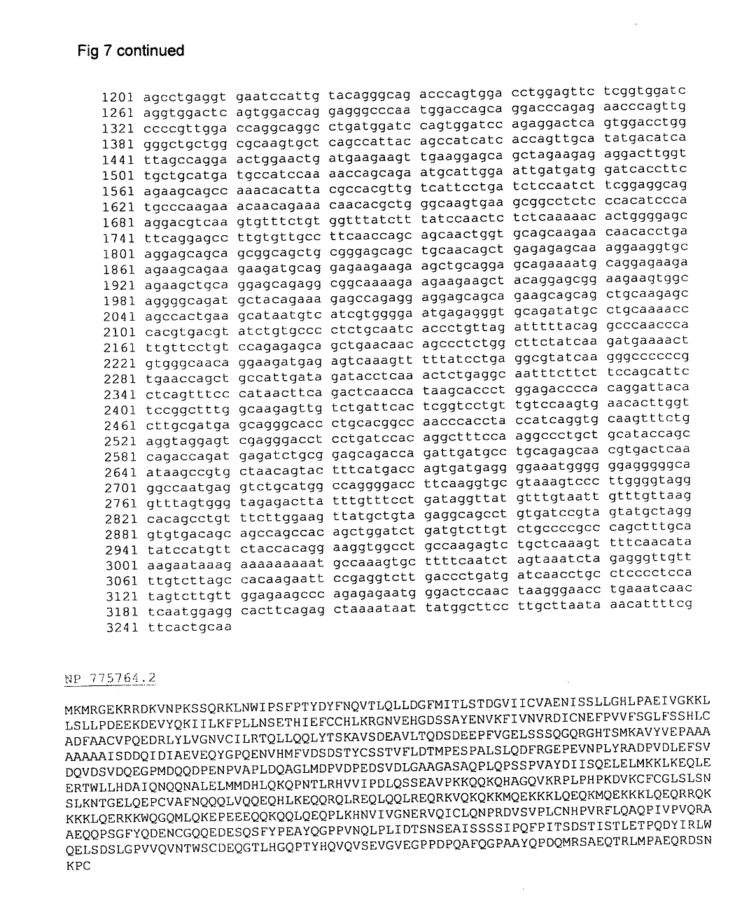

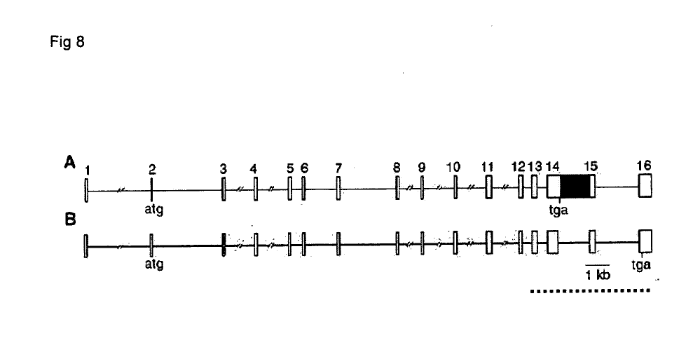

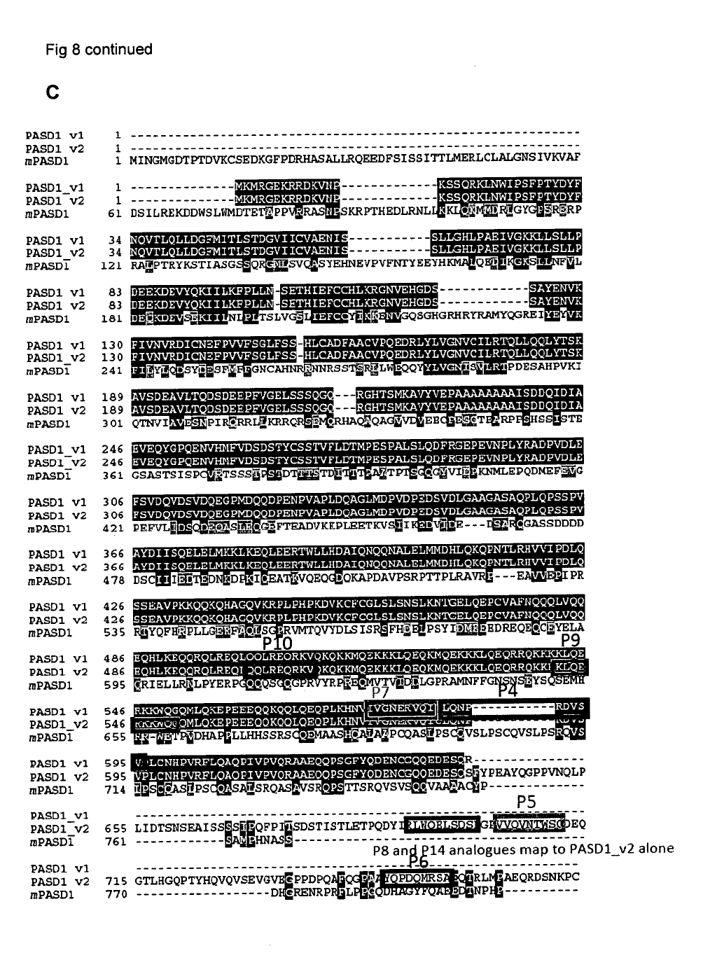

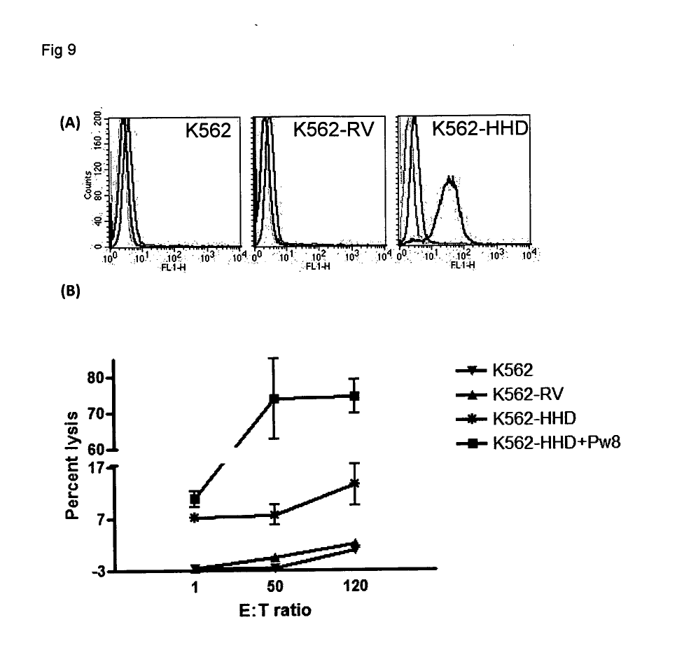

Mice were primed with pDOM.P14 vaccine and 28 days later boosted with the same. On day 56 mice were culled and the spleens stimulated with 1 μM of P8 peptide loaded and irradiated splenocytes on a weekly basis. IL-2 was given at each feed. Once CTL lines were seen to expand (a tripling of cell numbers in one week) they were used to target PASD1 positive, HLA-A2 positive or negative lines. We found that P14 lines could kill P8 peptide loaded K562-HHD+ cell lines (FIG. 6A, see also FIG. 9) and K562-HHD lines as compared to vector control K562 lines (FIG. 6B). In addition the P14 lines were very effective at killing the innately A2+PASD1+SW480 colon cancer cell line compared with the A2 negative PASD1+K562 cell line (FIG. 6C).

This is further illustrated in FIG. 9. The myeloid leukaemia (CML) human K562 cell line is PASD1 positive but MHC class I negative. Following transduction of the K562 cell line with the HHD-containing retrovirus (FIG. 9A), the ability of P14-specific CTL lines expanded from vaccinated mice to kill the human cells were investigated. Mice were primed with p.DOM-P14, splenocytes removed 14 days later and then stimulated ex vivo with P14. We showed in multiple experiments that CTL lines could kill P8 loaded K562-HHD cells (FIG. 9B) showing the capacity of these lines to kill target wt peptide. In addition, CTL lines showed detectable although lower levels of killing of K562-HHD cells in the absence of exogenous peptide loading (FIG. 9B), suggesting that the native P8 peptide was processed and presented from endogenously produced PASD1_v2.

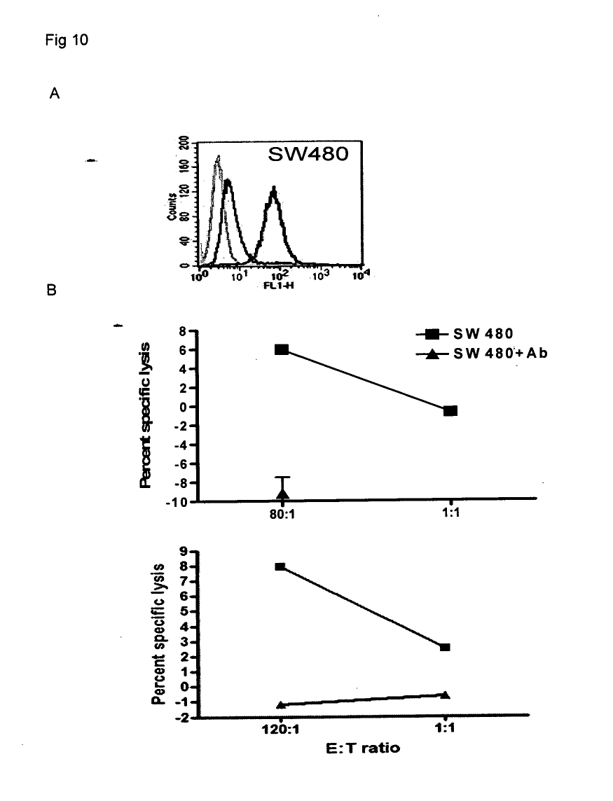

The inventors further examined a SW480 colon cancer cell line. This line is HLA class I positive (FIG. 10A) and PASD1 positive. The inventors found that a number of P14 lines were able to kill SW480 due to the endogenously processed and presented P8, despite the absence of HHD transduction (FIG. 10B). Mouse CD8+ cells do not interact with human MHC Class I, but the HHD mice have a transgenic human HLA-A2 molecule which their T cell can interact with. To achieve this, the T cells must be of high affinity. Use of a HLA-A2 blocking antibody inhibited MHC class I mediated target cell lysis (FIG. 10B) while the isotype control antibody did not. In summary, the killing by the P14 T-cell lines was HLA-A2 dependent, required CD8+ and was mediated via recognition of the naturally processed P8 epitope of endogenous PASD1_v2.

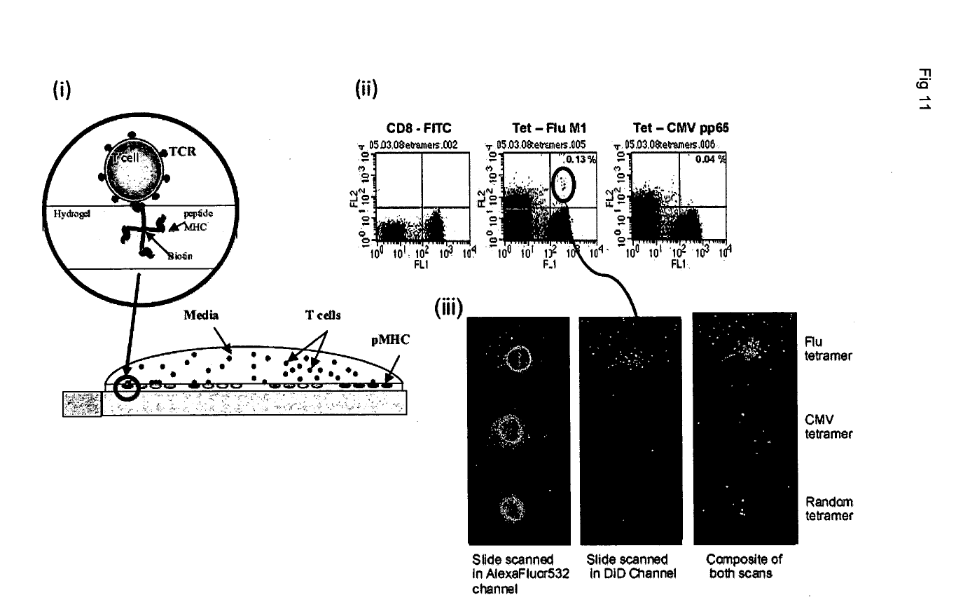

The inventors further used peptide-MHC microarrays, as described in^{42,43,} to further test whether AML patients had T cells which could recognize the P14 peptide on HLA-A2. In short, pMHC molecules were folded into tetramers using streptavidin alone or streptavidin conjugated to AlexaFluor 532 (Molecular Probes). Tetramers were spotted onto hydrogel slides using a contact deposition-type printer (Genetix), at a concentration of 0.5 mg/ml in 2% glycerol. Printed arrays were immobilised for 48 hours and stored at 4° C. until use. (FIG. 11 i) CD8+ T cells were negatively isolated from normal donor buffy coats obtained from National Blood Service UK or patient samples from the Department of Haematology, Southampton General Hospital following informed consent, using EasySep isolation kits. Cells were lipophillically dyed with DiD (Molecular Probes) according to the manufacturer's instructions. The selected array was warmed to room temperature and incubated with labelled CD8+ cells (10̂6/ml) in X-VIVO 15 for 20 minutes at 37° C. Unbound cells were washed away with warm X-VIVO. Excess culture medium was removed before slides were analysed on the ProScanArray (PerkinElmer). (FIG. 11 ii) FACS analysis was used to confirm T-cell populations recognising specific epitopes. Briefly, negatively isolated CD8+ T cells were labelled with CD8-FITC (FL1-H) and pMHC-SAPE (FL2-H) and analysed by flow cytometry using the FACScalibur™. The inventors showed that a minimum 0.7×10̂6 CD8+ cells (including controls) could be used to detect CMV and Flu specific populations in a HLA-A*0201 positive, Flu+M1, CMV pp65 negative sample. A small population of cells is visible in the upper right quadrant in the Flu M1 test while no background staining was observed in the upper right quandrant when CMV pp65 analysed. (FIG. 11 iii) On custom-made hydrogel slides CD8+ T cells from the same normal donor (shown stained red) are visible bound to the Flu M1 tetramer (shown in green) at the single cell level. Composites show the co-localisation of Flu-specific CD8+ T cells bound to tetramer spots from a HLA-A2 +, Flu M1 +, but not to the CMV pp65 or random tetramer negative control spots.

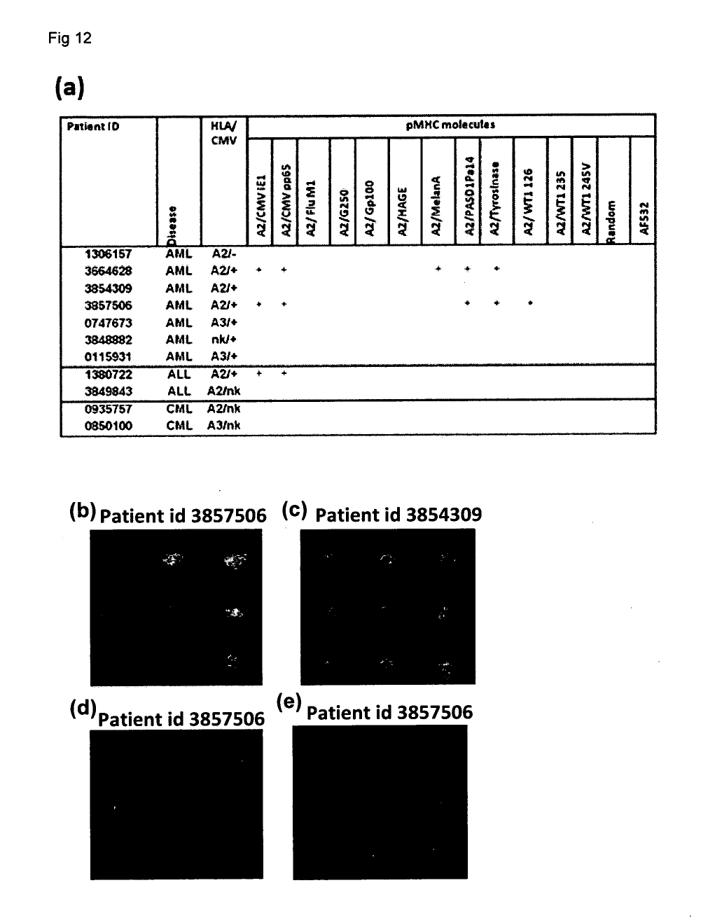

The inventors analysed 7 AML patients (FIG. 12). 4 of those 7 AML patients were A2 positive and of these 4, 2 had T cells which recognized the P14 epitope presented on HLA-A2. Both patients who were positive for P14 were also positive for CMV (IE1 and pp65). The inventors also analysed 2 ALL and 2 CML patients, 1 of each were HLA-A2 positive but neither had P14-specific T cells. Thus, two of four HLA-A2 positive AML patients had P14 specific T cells at presentation, which were detectable on the pMHC array despite no prior stimulation with P14 peptide ex vivo. The T cells in these samples were not expanded to increase the percentage of CD8+ T cells which can recognize P14. This shows that the tetramer array technique can detect PASD1 specific T cells in the peripheral blood of AML patients at disease presentation at a clinically relevant level (>0.01% of the total CD8+ population, which is comparable to FACS analysis) even in the absence of ex vivo T cell stimulation with peptide. The pMHC array technique allows examination of a lot of different T cell populations simultaneously in very small samples.

Patients may thus be screened prior to treatment to identify those patients that will benefit from immunotherapy which stimulates T cells specific for those epitopes. This method would identify patients who could benefit from P14-targetted therapy. The waning of T cell numbers indicate which other epitopes could be targeted. It is believed that patients with multiple T cell responses are more likely to respond well to chemotherapy (which instigates cell death, release of antigens to the immune system, and inflammation, necessary for effective T cell responses).

The present invention thus provides methods for predicting a subject's susceptibility for an immunotherapy based on epitopes/peptides of the invention. For example, using the methods described herein a subject can be identified as being likely to respond to PASD1 based therapy, in particular a therapy based on the peptides and epitopes described herein, such as for example but not limited to P14.

### Discussion

Through the analysis of seven modified peptides (analogues) the inventors have identified peptides which can induce effective immune responses in vivo and in vitro. From in vitro analysis using peptide loaded antigen presenting cells it was not clear whether P14 or P15 was more effective at inducing T cell immune responses. Once the inventors had prepared pDOM-epitope vaccines and immunised HHD mice it was clear that P14 was able to induce strong ELISpot and CTL responses against P14 and the wild type P8 peptide. The inventors have now shown that the wild type P8 sequence is processed and presented, and that CTL lines developed following the immunization of HHD mice with pDOM.P14, can kill human tumour cells which present endogenously processed and presented wild type peptide. P14 stimulated elevated levels of IFNγ production from T cells of normal donors and from patients with AML and colon cancer. The inventors have also shown that the modification of the P8 peptide to produce P14 was important for the induction of IFNγ ELISpot responses against both the P14 and P8 peptide, as well as CTL responses against leukaemia cells which were either peptide loaded or presenting endogenously processed antigen.

The pDOM.epitope vaccine design has allowed the inventors to determine accurately which of the modified epitopes can induce effective T cell responses against PASD1. In contrast to the in vitro MLRs using human samples, the data generated in HHD mice clearly show the effectivity of the peptides of the invention, in particular P14 (SEQ ID NO 21) at inducing effective T cell responses against the modified P14 peptide, the wild type P8 (SEQ ID NO 9) peptide and endogenously processed antigen. The human data reproducibly showed the improved effectivity of P14 peptide to induce T cell expansion and IFNγ secretion in HLA-A2+PASD1+AML patients and a colon cancer patient showing the wide ranging applicability of the PASD1 vaccine against haematological and solid cancers.

1.-63. (canceled)

64. An immunogenic peptide of 8 to 50 amino acids in length comprising at least one PASD1 epitope, wherein the epitope comprises the amino acid sequence of any one of SEQ ID NO: 1, 3, 5, 7, 9, 11, 13, 15, 17, 19, or 21 or a functional variant thereof.

65. The immunogenic peptide of claim 64, wherein the peptide is either 9 or 10 amino acids in length.

66. The immunogenic peptide of claim 64, wherein the peptide is capable of stimulating a T cell response, such as a cytotoxic T cell (CTL) response or a T helper (T_{H}) cell response.

67. The immunogenic peptide of claim 64, wherein the peptide comprises the amino acid sequence of any one of SEQ ID NO: 1, 3, 5, 7, 9, 11, or 13 and comprises at least one amino acid substitution.

68. A polyepitope string comprising at least a first PASD1 epitope of claim 64 and either a second PASD1 epitope having the amino acid sequence of any one of SEQ ID NO: 1, 3, 5, 7, 9, 11, 13, 15, 17, 19, or 21 or a functional variant thereof or an epitope of a different antigen.

69. A nucleic acid molecule encoding the peptide of claim 64, wherein the nucleic acid molecule optionally comprises the sequence of any one of SEQ ID NO: 2, 4, 6, 8, 10, 12, 14, 16, 18, 20, or 22.

70. An expression vector comprising the nucleic acid molecule of claim 69, wherein the vector is optionally a pDOM plasmid.

71. A coated particle comprising the peptide of claim 64.

72. A cell comprising the peptide of claim 64, wherein optionally the cell is an antigen presenting cell (APC) or a dendritic cell (DC).

73. A T cell or a T cell line which specifically recognizes the PASD1 epitope of claim 64, wherein optionally the T cell is a cytotoxic T cell (CTC) or a T helper (T_{H}) cell.

74. An agent capable of specifically binding the peptide or PASD1 epitope of claim 64, wherein optionally the agent comprises a T cell receptor or an antibody.

75. A monomeric, tetrameric or pentameric complex comprising a multivalent major histocompatibility complex (MHC) molecule presenting the peptide or PASD1 epitope of claim 64.

76. A composition comprising the peptide or PASD1 epitope of claim 64 and a pharmaceutically acceptable carrier or diluent.

77. A vaccine comprising the peptide or PASD1 epitope of claim 64 and optionally further comprising an adjuvant and/or an additional TAA peptide.

78. A method of inducing an antigen-specific immune response in a subject in need thereof comprising administering an effective amount of the immunogenic peptide of claim 64 to said subject.

79. The method of claim 78, wherein said method comprises prophylactic or therapeutic vaccination.

80. The method of claim 78, wherein said method comprises treating cancer in said subject.

81. The method of claim 80, wherein said method further comprises administering chemotherapy and/or radiotherapy and/or immunotherapy to said subject.

82. The method of claim 80, wherein said cancer is selected from multiple myeloma, mantel cell lymphoma, Hodgkin's lymphoma, T cell lymphomas, follicular lymphoma, Burkitt's lymphoma, T cell rich B cell lymphoma, diffuse large B-cell lymphoma (DLBCL), chronic myeloid leukaemia, myelodysplastic syndrome (MDS), acute myeloid leukemia (AML), melanoma, lung cancer, breast cancer, gastric cancer, kidney cancer, prostate cancer, ovarian cancer, uterine cancer, colorectal cancer, liver cancer, head and neck cancer, adenocarcinoma of the colon, a hematologic malignancy, acute myeloid leukaemia, chronic myeloid leukaemia (CML), and myelodysplastic syndrome (MDS).

83. A method of predicting the susceptibility of a subject to a treatment for cancer comprising testing a sample obtained from said subject for the presence of:
(a) a T cell or T cell line that recognizes the peptide or PASD1 epitope of claim 64;
(b) a peptide comprising the PASD1 epitope of claim 64;
(c) an APC or tumour cell presenting the PASD1 epitope of claim 64 on an MHC class I molecule;
(d) a T-cell receptor (TCR) that recognizes the peptide or PASD1 epitope of claim 64;
(e) a T cell activated against the peptide or PASD1 epitope of claim 64; or
(f) a peptide-specific T cell identified using a pMHC array;
wherein detection of any one of features (a) to (f) indicates the susceptibility of said subject for said treatment.

84. The method of claim 83, wherein said detection comprises using a monomeric, tetrameric, or pentameric complex comprising a multivalent major histocompatibility complex (MHC) molecule presenting the peptide or PASD1 epitope of claim 64 to detect the T cell of (a), (e), or (f).

85. A method of generating an immunogenic variant peptide comprising:
(i) obtaining a parent peptide comprising at least one copy of a subsequence of PASD1 comprising any one of SEQ ID NO: 1, 3, 5, 7, 9, 11, or 13,
(ii) modifying the subsequence of the parent peptide by substitution, deletion, or insertion of one or more amino acids, and
(iii) testing the variant peptide of (ii) for immunogenicity.

86. A method of detecting and/or staging a cancer comprising testing a sample obtained from a subject for the presence of:
(a) a T cell or T cell line specific for the peptide or PASD1 epitope of claim 64;
(b) the peptide or PASD1 epitope of claim 64;
(c) an APC or tumour cell presenting the PASD1 epitope of claim 64 on an MHC I molecule, or
(d) a TCR that recognizes the peptide or PASD1 epitope of claim 64;
(e) a T cell activated against the peptide or PASD1 epitope of claim 64; or
(f) a peptide-specific T cell identified using a pMHC array.

87. A method of monitoring an anti-PASD1 immune response in a subject comprising detecting in a sample obtained from the subject the presence of:
a) the peptide or PASD1 epitope of claim 64;
b) a T cell or T cell line specific for the peptide or PASD1 epitope of claim 64; or
c) a T cell receptor that recognizes the peptide or PASD1 epitope of claim 64;
wherein the presence of said peptide or epitope, said T cell or T cell line, or said T cell receptor indicates said anti-PASD1 immune response in said subject.
